(19) United States
(12) Patent Application Publication
(10) Pub. No.: US 2026/0241047 A1
(43) Pub. Date: Aug. 20, 2026

(54) FIBROBLAST ACTIVATION PROTEIN-TARGETING RADIOPHARMACEUTICALS AND USES THEREOF

(71) Applicants: Nuclidium AG, Basel (CH); University of Basel, Basel (CH)

(72) Inventors: Leila Jaafar-Thiel, Basel (CH); Francesco De Rose, Basel (CH); Melpomeni Fani, Basel (CH); Jacopo Millul, Basel (CH)

(21) Appl. No.: 19/375,117

(22) Filed: Oct. 30, 2025

Related U.S. Application Data
(63) Continuation of application No. PCT/US2025/021148, filed on Mar. 24, 2025.
(60) Provisional application No. 63/568,863, filed on Mar. 22, 2024.

(51) Int. Cl. A61K 51/04; A61K 9/00; A61K 31/375; A61K 47/02

(52) U.S. Cl. CPC A61K 51/0497; A61K 9/0019; A61K 31/375; A61K 47/02; A61K 51/0455; A61K 51/0459; A61K 2123/00

(57) ABSTRACT

The present disclosure relates to pharmaceutical compositions comprising a [⁶¹Cu]Cu-labeled fibroblast activation protein (FAP) inhibitor-NODAGA conjugate. The present disclosure also relates to methods of imaging subjects suspected of having or diagnosed with a FAP-expressing cancer or tumor by administering the pharmaceutical compositions described herein and generating one or more radiographic images of the subject.

### 1. BACKGROUND

Fibroblast activation protein (FAP) is a transmembrane glycoprotein expressed on activated fibroblasts such as cancer-associated fibroblasts (CAFs), a primary component of tumor microenvironment. Structurally, FAP is a type II transmembrane glycoprotein consisting of 760 amino acids. FAP is a serine protease, and unlike other members of the dipeptidyl peptidase (DPP) family, it has both endopeptidase and exopeptidase activity, which enable it to cleave gelatin and type I collagen and play an important role in extracellular matrix (ECM) remodeling. CAFs with FAP expression are found in various neoplasms, particularly epithelial cancers, and in malignancies with a strong desmoplastic reaction such as breast (ductal and lobular), colorectal, pancreatic, and lung cancer. Overall, a high degree of FAP expression is associated with tumor aggressiveness and poor prognosis (Cohen, S. J., et al., Pancreas 2008, 37, 154-158). The negligible expression of FAP in normal healthy adult tissues makes it an attractive target for oncological imaging and therapy (Lindner, T., et al., EJNMMI Radiopharm. Chem. 2019, 4, 16.).

In sarcomas, FAP expression was found in both stromal and tumor/nonstromal cells (Koerber S. A. et al., EJNMMI 2021, 48). Fibroblast activation and consequent FAP expression is also increased in non-neoplastic diseases as fibrosis, rheumatoid arthritis, atherosclerosis.

FAP overexpression has been targeted with small-molecule FAP inhibitors (aka “FAPIs”) with an N-(4-quinolinoyl)-Gly-(2-cyanopyrrolidine) scaffold, first developed at the University of Antwerp (Hansen, K., et al., ACS Med. Chem. Lett. 2013, 4, 491-496; Jansen, K., et al., J. Med. Chem. 2014, 57, 3053-3074). This scaffold was modified to develop FAPI-01 and FAPI-02 as the first quinoline-based FAPIs, which were radiolabeled with ¹²⁵I and ⁶⁸Ga/¹⁷⁷Lu, respectively. Further attempts at improving tumor retention led to the development of FAPI-46 (Loktev, A., et al., J. Nucl. Med. 2019, 60, 1421-1429). WO 2019/154886 describes FAP inhibitors, FAP inhibitor-chelator constructs, radiolabeled FAP inhibitor-chelator constructs useful for diagnosis or treatment of diseases characterized by overexpression of FAP, e.g., cancer.

Because FAP overexpression is not limited to CAFs, the use of FAP inhibitors in combination with positron emission tomography-computed tomography (PET-CT) may find application in a wide range of non-oncological pathological states, e.g., inflammatory, infectious, and immune pathologies. FAP overexpression has also been associated with cardiovascular diseases, liver fibrosis and cirrhosis, arthritic disorders (e.g., rheumatoid arthritis), IgG4-related disease, pulmonary fibrosis and interstitial lung disease, Crohn's disease, tuberculosis, sarcoidosis, and periprosthetic joint infections (Chandekar, K. R., et al., FAPI PET/CT Imaging—An Updated Review. Diagnostics 2023, 13, 2018).

Radiolabeled FAP-targeting small ligands, e.g., [⁶⁸Ga]Ga-FAPI-46 and [⁶⁸Ga]Ga-OncoFAP, have recently been introduced and demonstrated high and fast uptake in a variety of cancers and rapid clearance from the majority of healthy organs in human PET imaging (Backhaus P., et al., EJNMMI, 2022, 49(6):1822-1832).

Use of [⁶⁸Ga]Ga-labelled imaging agents is hampered by (i) the expense and limited production capacity of ⁶⁸Ge/⁶⁸Ga-generators, (ii) the short half-life of ⁶⁸Ga (t1/2=1.13 h), which prevents the shipment of the radiotracer from central producers to smaller centers that are located beyond 2 hours' distance, and (iii) the high-energy of ⁶⁸Ga (Emax=1.9 MeV), which limits spatial resolution of reconstructed PET imaging.

Use of FAPI-based imaging agents is hampered by the quick washout from target tissues.

There is a need for improved methods of generating radiographic images of subjects with FAP-expressing tumors for the diagnosis, monitoring, and ultimately, treatment of cancer patients.

### 2. SUMMARY

In one aspect, the present disclosure provides a pharmaceutical composition comprising:
(a) a radiotracer that has one of the following structures

| Radiotracer | Structure |
|---|---|
| ⁶¹Cu-NODAGA-1 | 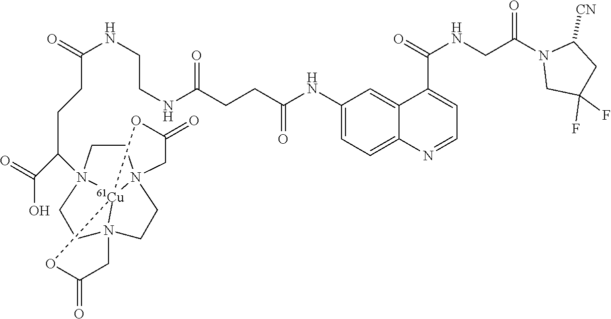 |
| ⁶¹Cu-NODAGA-2 | 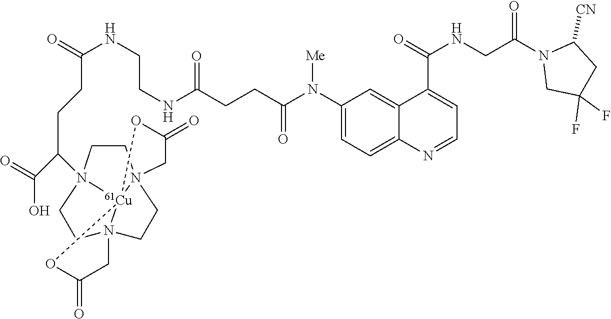 |
| ⁶¹Cu-NODAGA-3 | 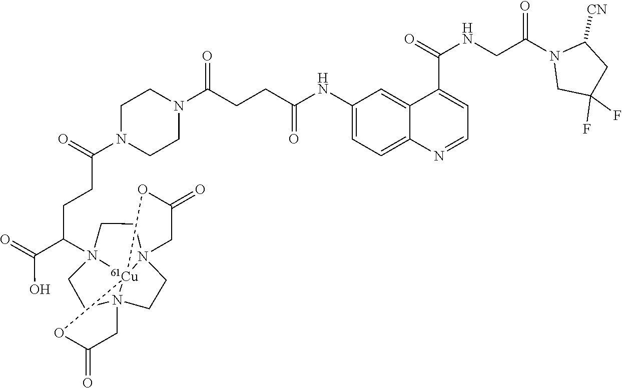 |
| ⁶¹Cu-NODAGA-4 | 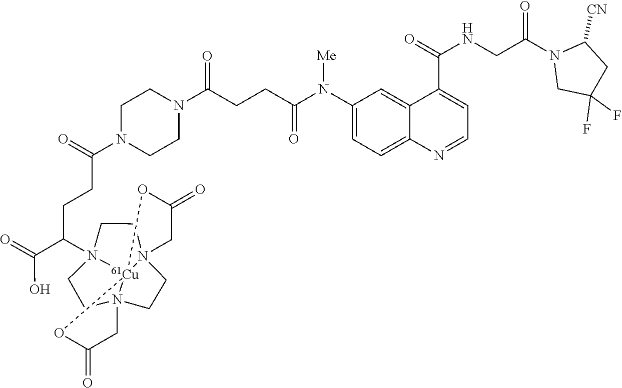 |

or is a pharmaceutically acceptable salt thereof,
(b) a radiolytic inhibitor, such as ascorbic acid, and
(c) an aqueous vehicle;
   wherein the pharmaceutical composition is optionally characterized by one or more of:
a [⁶¹Cu]Cu radionuclidic purity at end of synthesis of ≥97%,
a radiocobalt activity content at end of synthesis of ≤0.05%,
a ^{110m}Ag specific activity ≤0.1 Bq/g,
a ^{108m}Ag specific activity ≤0.1 Bq/g, or
a ¹⁰⁹Cd specific activity ≤0.1 Bq/g.

In another aspect, the present disclosure provides a method for imaging a subject comprising:
(a) administering an effective amount of a pharmaceutical composition to a subject suspected of having or diagnosed with a disorder associated with expression of FAP (e.g., a FAP-expressing cancer or tumor, fibrosis, rheumatoid arthritis, and atherosclerosis), wherein the pharmaceutical composition comprises:
   i. a radiotracer that has one of the following structures

| Radiotracer | Structure |
|---|---|
| ⁶¹Cu-NODAGA-1 | 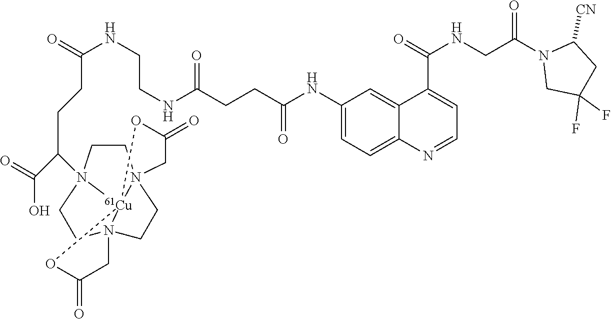 |
| ⁶¹Cu-NODAGA-2 | 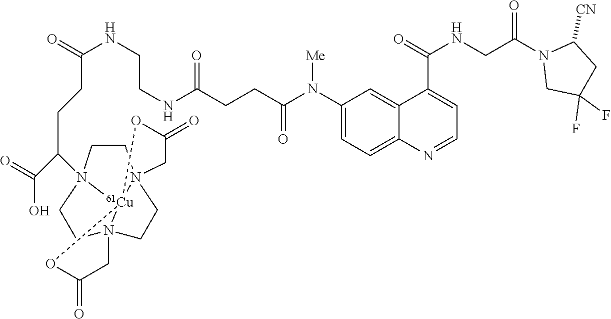 |
| ⁶¹Cu-NODAGA-3 | 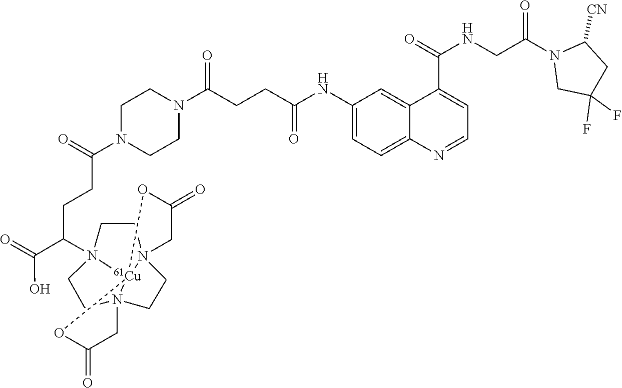 |
| ⁶¹Cu-NODAGA-4 | 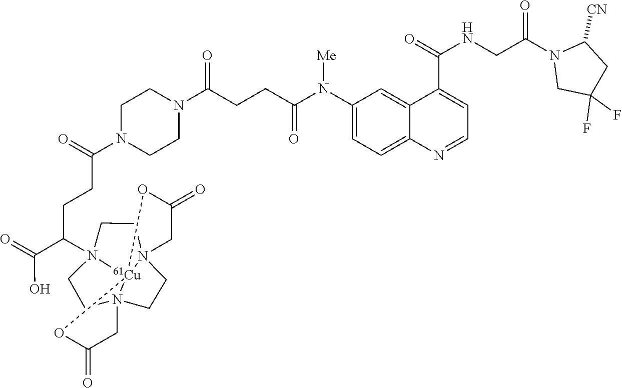 |

or is a pharmaceutically acceptable salt thereof, and
ii. a radiolytic inhibitor, such as ascorbic acid;
wherein the pharmaceutical composition is optionally characterized by one or more of:
   a [⁶¹Cu]Cu radionuclidic purity at end of synthesis of ≥97%,
   a radiocobalt activity content at end of synthesis of ≤0.0500,
   an amount of ^{110m}Ag≤0.1 Bq/g,
   an amount of ^{108m}Ag≤0.1 Bq/g, or
   an amount of ¹⁰⁹Cd≤0.1 Bq/g; and
(b) generating one or more radiographic images of the subject.

In another aspect, the present disclosure provides a method for determining a subject's response to a cancer treatment comprising:
(a) administering an effective amount of a pharmaceutical composition to a subject diagnosed with a FAP-expressing cancer or tumor at an earlier time point and at a later time point, wherein the pharmaceutical composition comprises:
   i. a radiotracer that has one of the following structures

| Radiotracer | Structure |
|---|---|
| 61Cu-NODAGA-1 | 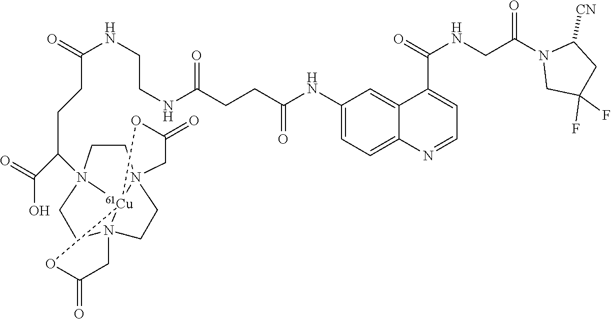 |
| ⁶¹Cu-NODAGA-2 | 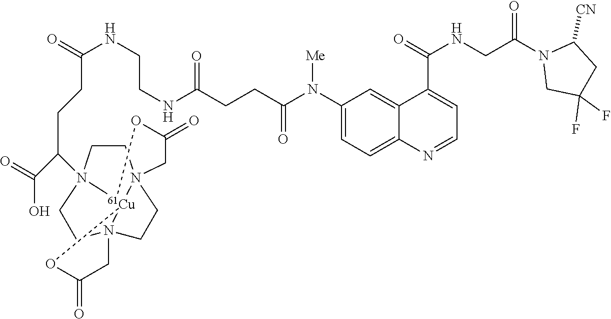 |
| ⁶¹Cu-NODAGA-3 | 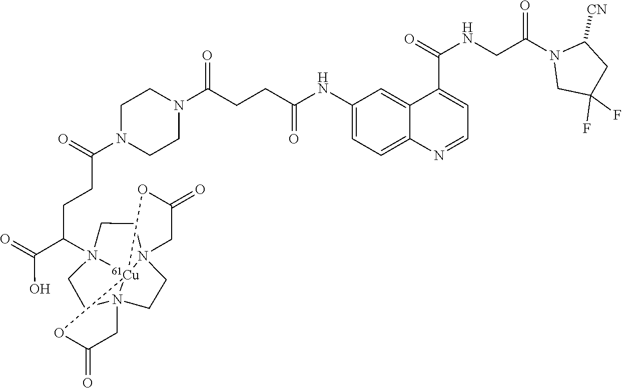 |
| ⁶¹Cu-NODAGA-4 | 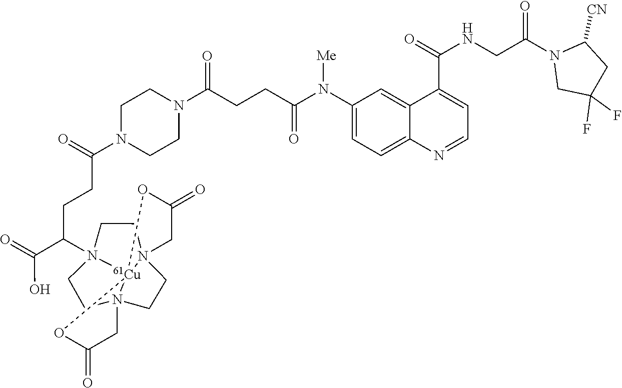 |

or is a pharmaceutically acceptable salt thereof, and
ii. a radiolytic inhibitor, such as ascorbic acid;
wherein the pharmaceutical composition is optionally characterized by one or more of:
   a [⁶¹Cu]Cu radionuclidic purity at end of synthesis of ≥97%,
   a radiocobalt activity content at end of synthesis of ≤0.05%,
   a ^{110m}Ag specific activity ≤0.1 Bq/g,
   a ^{108m}Ag specific activity ≤0.1 Bq/g, or
   a ¹⁰⁹Cd specific activity ≤0.1 Bq/g;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

### 3. BRIEF DESCRIPTION OF THE DRAWINGS

These and other features, aspects, and advantages of the present disclosure will become better understood with regard to the following description, and accompanying drawings, where:

FIG. 1 illustrates with increasing magnification homogenous nickel coating having durable adhesion to a niobium coin upon completion of electroplating, as evaluated using a DINOLite digital microscope. Panel A, 20× magnification; panel B, 50× magnification; panel C, 250× magnification.

FIG. 2 shows samples of the coin provided according to the present disclosure with nickel deposited in the center of a niobium backing.

FIG. 3 displays the analysis of ⁶¹Cu purity of [⁶¹Cu]CuCl₂ solution obtained by irradiation of ^{nat}Ni on Nb backing with deuteron beam at 8.4 MeV for 3 h at 50 μA. The curved line corresponds to reduction in % purity of ⁶¹Cu over time and the bars correspond to radiocobalt activity over time.

FIG. 4 displays an analysis of ⁶¹Cu purity of [⁶¹Cu]CuCl₂ solution obtained by irradiation of ⁶⁰Ni on Nb backing with a deuteron beam at 8.4 MeV for 3 h at 50 μA. The curved line corresponds to the reduction in % purity of ⁶¹Cu over time, and the bars correspond to radiocobalt activity over time.

FIG. 5 presents the specific activity of detected impurities in [⁶¹Cu]CuCl₂ solutions produced according to various methods. The ext. coin (Ag, natNi) data was generated by irradiation of a commercially available ^{nat}Ni target on Ag backing. The (Nb, natNi) and (Nb, Ni-61) data were generated based on irradiation of Ni targets (natural and isotopically enriched in ⁶¹Ni, respectively) electroplated according to the present disclosure on high-purity Nb backing. The specific activity was assessed by gamma spectrometry and reported in Bq/g. The data shows that silver and cobalt isotopes are significantly reduced in the [⁶¹Cu]CuCl₂ solution produced by irradiation of Ni targets electroplated according to the present disclosure on high-purity Nb backing.

FIG. 6 shows the significant reduction in the sum of radionuclidic impurities present in a [⁶¹Cu]CuCl₂ solutions produced according to various methods. The ext. coin (Ag, natNi) data was generated based on irradiation of a commercially available natNi target on Ag backing. The (Nb, natNi) and (Nb, Ni-61) data were generated based on irradiation of Ni targets (natural and isotopically enriched in ⁶¹Ni, respectively), electroplated according to the present disclosure on high-purity Nb backing. The radionuclidic impurities were determined by gamma spectrometry and reported in Bq/g (summed radionuclidic impurities). The presented data highlight in particular the reduction of overall impurities in the [⁶¹Cu]CuCl₂ solution when produced in accordance with the present disclosure.

FIG. 7 illustrates the sustained high radionuclidic purity of a [⁶¹Cu]CuCl₂ solution produced according to the present disclosure compared to a commercially available ^{nat}Ni target on a Ag backing (ext. coin (Ag, natNi)). The (Nb, natNi) and (Nb, Ni-61) coins were prepared by electrodeposition according to the present disclosure on high-purity Nb backing. The data was generated using gamma spectrometry and reported in Bq/g providing the summed radionuclidic purities at t=0 h and at t=12 h. The presented data highlight the superior quality of the [⁶¹Cu]CuCl₂ solution when produced by irradiation of Ni targets electroplated according to the present disclosure on high purity Nb backing, where the purity after 12 hours is still well above the purity limits set by pharmacopeia for similar radionuclides for medical use.

FIG. 8 displays chemical impurities, as measured by ICP-MS, of the [⁶¹Cu]CuCl₂ solution when produced by bombardment of ^{nat}Ni vs. ⁶¹Ni when produced by irradiation of Ni targets electroplated according to the present disclosure on high-purity Nb backing.

FIG. 9 shows the partition coefficient (log D _{PBS/octanol, pH=7.4}) of ⁶¹Cu or ⁶⁸Ga-labeled conjugates. From left to right: [⁶¹Cu]Cu-NODAGA-1, [⁶¹Cu]Cu-NODAGA-3, [⁶¹Cu]Cu-NODAGA-2, [⁶¹Cu]Cu-NODAGA-4, [⁶⁸Ga]Ga-FAPI-46, and [⁶¹Cu]Cu-NODAGA-FAPI-46.

FIG. 10 shows the inhibition (IC₅₀) of ^{nat}Cu-labeled fluorescent conjugates (fluorescence units vs concentration).

FIG. 11, panels A-D, show cellular uptake of cell surface (cell membrane bound) and internalized fractions of [⁶¹Cu]Cu-NODAGA-1 (panel A), [⁶¹Cu]Cu-NODAGA-3 (panel B), [⁶¹Cu]Cu-NODAGA-2 (panel C), and [⁶¹Cu]Cu-NODAGA-4 (panel D). The values are expressed as % of the applied activity and refer to the specific uptake calculated after subtracting the non-specific values (measured in the presence of the non-FAP expressing cell line HT-1080.wt) from the total values (specific=total−non-specific).

FIG. 12 shows cellular uptake of cell surface (cell membrane bound) and internalized fractions of [⁶¹Cu]Cu-NODAGA-FAPI-46. The values are expressed as % of the applied activity and refer to the specific uptake calculated after subtracting the non-specific values (measured in the presence of the non-FAP expressing cell line HT-1080.wt) from the total values (specific=total−non-specific).

FIG. 13 shows the saturation binding of ⁶¹Cu-labeled conjugates on isolated HEK-293-hFAP membranes.

FIG. 14, panels A and B, show the biodistribution profiles of [⁶¹Cu]Cu-NODAGA-FAPI-46 (panel A) and [⁶⁸Ga]Ga-FAPI-46 (panel B) in HT-1080.hFAP tumor-bearing mice at 1 hour and 4 hours following administration.

FIG. 15, panels A and B, show the tumor-to-organ ratios of [⁶¹Cu]Cu-NODAGA-FAPI-46 (panel A) and [⁶⁸Ga]Ga-FAPI-46 (panel B) in HT-1080.hFAP tumor-bearing mice at 1 hour and hours following administration.

FIG. 16, panels A-B, show biodistribution profiles of [⁶¹Cu]Cu-NODAGA-1 (panel A) and [⁶¹Cu]Cu-NODAGA-3 (panel B), in HT-1080.hFAP tumor-bearing mice at 1 hour and 4 hours following administration.

FIG. 17, panels A-B, show biodistribution profiles of [⁶¹Cu]Cu-NODAGA-2 (panel A) and [⁶¹Cu]Cu-NODAGA-4 (panel B) in HT-1080.hFAP tumor-bearing mice at 1 hour and hours following administration.

FIG. 18, panels A-B, show the tumor-to-organ ratios of [⁶¹Cu]Cu-NODAGA-1 (panel A) and [⁶¹Cu]Cu-NODAGA-3 (panel B), in HT-1080.hFAP tumor-bearing mice at 1 hour and 4 hours following administration.

FIG. 19, panels A-B, show the tumor-to-organ ratios of [⁶¹Cu]Cu-NODAGA-2 (panel A), and [⁶¹Cu]Cu-NODAGA-4 (panel B) in HT-1080.hFAP tumor-bearing mice at 1 hour and 4 hours following administration.

FIG. 20 shows the dynamic PET/CT scans of [⁶¹Cu]Cu-NODAGA-1 and [⁶¹Cu]Cu-NODAGA-3 in mice bearing FAP-positive xenografts.

FIG. 21, panels A and B, show the dynamic PET/CT scans of [⁶¹Cu]Cu-NODAGA-2 (panel A) and [⁶¹Cu]Cu-NODAGA-4 (panel B) in mice bearing FAP-positive xenografts.

FIG. 22, panels A and B, show the dynamic PET/CT scans of [⁶¹Cu]Cu-NODAGA-FAPI-46 (panel A) and [⁶⁸Ga]Ga-FAPI-46 (panel B) in mice bearing FAP-positive xenografts.

FIG. 23, panels A and B, show SUV PET imaging of [⁶¹Cu]Cu-NODAGA-2 and [⁶¹Cu]Cu-NODAGA-4 (1 h and 4 h) (panel A) and [⁶¹Cu]Cu-NODAGA-FAPI-46 vs ⁶⁸Ga-FAPI-46 (1 h and 4 h for [⁶¹Cu]Cu-NODAGA-FAPI-46 and 1 h only for [⁶⁸Ga]Ga-FAPI-46) (panel B).

FIG. 24, panels A-B, illustrate the dynamic PET/CT scans of [⁶¹Cu]Cu-NODAGA-F1 (panel A) and [⁶¹Cu]Cu-NODAGA-F3 (panel B) in dual HT1080.hFAP and HT1080.wt tumor-bearing mice within 1 hour.

FIG. 25 shows AMA test plot obtained plotting at x-axis (logarithmic) the nmol of titrating chelator and at y-axis the % of complexation obtained via radio-TLC.

FIG. 26 shows experimental point corresponding to the lowest value of nmol of chelator in correspondence of which ≥95% complexation was achieved (black arrow).

### 4. DETAILED DESCRIPTION

### 4.1. Definitions

When describing the embodiments of the present disclosure, the following terms, if present, have the following meanings, unless otherwise indicated. If not otherwise defined, terms have their customary meaning in the relevant art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, “absorbed dose” refers to the amount of radiation absorbed by an object (e.g., an organ or tissue) or a person. The gray (Gy) is the SI unit of absorbed dose and is defined as the absorption of one joule of energy, in the form of ionizing radiation, per kilogram of matter, i.e., one gray=1 J/kg².

As used herein, “activity” or “radioactivity” refers to a physical quantity defined as the number of radioactive transformations per second that occur in a particular compound or composition that contains one or more radioactive substances. The unit of activity used herein is the becquerel (Bq), which is defined equivalent to reciprocal seconds (1/seconds or s⁻¹).

As used herein, “activity concentration” refers to the total amount of radioactivity per unit volume. In certain embodiments, activity concentration is expressed in Bq/L or magnitudes thereof (e.g., MBq/mL).

As used herein, “radiocobalt activity content” refers to the ratio, expressed as a percentage, of the radioactivity of radiocobalt species in a composition to the total radioactivity of the composition.

As used herein, “end of synthesis” refers to completion of radiolabeling NODAGA-1, NODAGA-2, NODAGA-3, or NODAGA-4 with [⁶¹Cu]CuCl₂ and subsequent preparation of the pharmaceutical composition, e.g., dilution with saline solution.

As used herein, “effective amount,” “pharmaceutically effective amount,” or “therapeutically effective amount” mean a sufficient amount of the pharmaceutical composition to provide the desired utility when administered to a subject. In certain embodiments, an effective amount includes an amount of pharmaceutical composition sufficient to generate an image of subject. In certain embodiments, an effective amount includes an amount of pharmaceutical composition sufficient to diagnose a disease in a subject. It is understood that for any given case, an appropriate “effective amount” can be determined by one of ordinary skill in the art using routine experimentation. For example, when administered in clinic, such pharmaceutical compositions will contain an amount of active ingredient effective to achieve the desired result (e.g., imaging cancerous tissue).

As used herein, “effective dose” refers to the internationally accepted central radiological risk metric quantity. The calculation of effective dose can be seen as a three-step process. First, the mean absorbed doses to organs and tissues are determined in gray (Gy; J kg⁻¹). Second, the absorbed doses are converted to equivalent doses in sievert (Sv) using tissue/organ radiation weighting factors (wR). The weighting factors were computed from radiation epidemiological data, and take into consideration that some organs (e.g., breasts, stomach, and, lungs) are more radiosensitive than others (e.g., brain and skin). The summing of organ/tissue equivalent doses, each weighted by the appropriate tissue weighting factor (wT), gives the effective dose, in Sv or Sv/MBq (i.e., the effective dose received per unit of activity administered). The effective dose is therefore a risk metric, and not a dosimetric quantity per se.

As used herein, “molar activity” refers to the amount of radioactivity (e.g., number of nuclear disintegrations per second) per unit mole of the radiolabeled compound. In certain embodiments, molar activity is expressed in Bq/mol, e.g., MBq/nmol and is used where the molecular weight of the labelled material is known. The “apparent molar activity” (apparent Am) takes into account the amounts of the radiolabeled (compound A*) and non-radiolabeled radiotracer (compound A), radiolabeled impurities (compound B*) and non-radiolabeled impurities (compound B) and remaining precursor (compound C) present expressed in mol (or μmol). See, e.g., Luurtsema, G., et al. “EANM guideline for harmonization on molar activity or specific activity of radiopharmaceuticals: impact on safety and imaging quality.” EJNMMI Radiopharm. Chem. 6, 34 (2021).

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium, and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutical composition” refers to a composition suitable for administration to a subject that comprises the radiotracer and one or more pharmaceutically acceptable excipients. A “pharmaceutically acceptable excipient”, as used herein refers to for example, pharmaceutically, physiologically, acceptable organic or inorganic carrier substances suitable for intravenous administration that do not deleteriously react with the radiotracer or salt thereof. Exemplary pharmaceutical excipients are known to those of skill in the art.

As used herein, “positive predictive value” or PPV refers to a given method's precision in identifying cancerous lesions/tumors. A method that has a high PPV is more desirable. Mathematically, PPV is the number of true positives (TP) divided by the sum of TP and false positives (FP). In other words, PPV is TP/(TP+FP).

As used herein, “peptide receptor radionuclide therapy” (PRRT) refers to the use of one or more radiotherapeutic agents for the delivery of targeting radiation, usually by a chelated radionuclide, to a specific tissue or receptor associated with cancer. The targeting and selection of certain tissues over another is accomplished by specific peptide hormone receptors expressed or overexpressed on the surface of cancerous biomass.

As used herein, “radiochemical purity” refers to the ratio, given as a percent, of radioactivity from the radionuclide in the pharmaceutical composition (e.g., the desired radionuclide that is chelated in a radiotracer as described herein) to the total radioactivity of the composition that comprises the radiotracer. The majority of the radioactive isotope is attached to the tracer construct and is not free or attached to another chemical entity as these forms may have a different biodistribution. Radiochemical purity (RCP) measurements establish the content of impurities labelled with the same radionuclide used to prepare a radiopharmaceutical, but with a different chemical form.

Radiochemical purity is determined according to methods well known to those of skill in the art, e.g., radio-HPLC and/or iTLC. As is understood in the art, determination of radiochemical purity is not strictly quantitative, and it is calculated as the ratio between the peak area of the desired radiotracer and the overall area of all the detected peaks in the radiochromatogram (corrected for decay). The instrument used to determine radiochemical purity with HPLC (radio-HPLC) is a radiometric detector (radiodetector), which has an in-line detector connected in series with a UV or other physicochemical detector. The radiometric detector can be a Geiger-Müller probe, a scintillation detector, or a PIN diode. As compared with radio-HPLC it has the big advantage that all applied radioactivity is detected and there are no concerns with recovery. The radiochemical purity is reported at a particular time point, e.g., a certain time after end of bombardment (EoB) or end of synthesis (EoS) (“synthesis” being, e.g., radiolabeling with the copper radionuclide). In certain embodiments, a radiotracer radiochemical purity is measured at 3 hours after end of synthesis of the radiotracer, at 6 hours after end of synthesis of the radiotracer, at 9 hours after end of synthesis of the radiotracer, or at 12 hours after end of synthesis of the radiotracer.

As used herein, “radionuclidic purity” refers to the ratio, expressed as a percentage, of the radioactivity of a particular radionuclide to the total radioactivity of the sample, e.g., the starting material used to prepare a radiotracer. In certain embodiments, radionuclidic purity can be determined by high resolution gamma spectroscopy (e.g., high-purity germanium (HPGe) detector) on a sample after expiration, e.g. >8 hours or >3 weeks) and is then extrapolated (e.g., using the TENDL-2019 database according to procedures well known in the art), and reported herein as the value at the end of synthesis (e.g., EoB+2 hours) of the radionuclide.

As used herein, “radionuclidic impurities” refer to all non-⁶¹Cu radionuclides in a composition.

As used herein, “radiotracer” refers to a compound of the present disclosure comprising a radionuclide or radioisotope. It is understood herein that when a compound, e.g., a radiotracer, is described as comprising a particular radioisotope or radionuclide (e.g., ⁶¹Cu) that the compound is isotopically enriched in that isotope at the indicated position.

As used herein, “sensitivity” refers to the ability of a given method to accurately identify cancerous lesions/tumors. A method that has high sensitivity is more desirable. Mathematically, sensitivity is the number of true positives (TP) divided by the sum of the number of TPs and false positives (FP). In other word, sensitivity (Se)=TP/(TP+FN).

As used herein, “specific activity” refers to the activity of a radionuclide per unit mass or per unit volume. In certain embodiments, specific activity refers to the activity of a radionuclide per unit mass of the radiotracer. In certain embodiments, specific activity refers to the activity of a radionuclide per unit mass of the [⁶¹Cu]CuCl₂ solution used to radiolabel NODAGA-1, NODAGA-2, NODAGA-3, or NODAGA-4. In certain embodiments, specific activity refers to the activity of a radionuclide per unit volume of the [⁶¹Cu]CuCl₂ solution used to radiolabel NODAGA-1, NODAGA-2, NODAGA-3, or NODAGA-4. In certain embodiments, specific activity is measured at EoS.

As used herein, “subject” refers to the person or organism to which the composition is, or is intended to be, administered. As such, subjects of the present disclosure may include but are not limited to mammals, e.g., humans and other primates, such as chimpanzees and other ape and monkey species. In preferred embodiments the subject are humans. The term subject includes a person or organism of any age, weight, or other physical characteristic, including an adult, an adolescent, a child, an infant or a newborn.

### 4.2. Pharmaceutical Compositions

In one aspect, the present disclosure provides pharmaceutical compositions comprising a radiotracer that has one of the following structures

| Radiotracer | Structure |
|---|---|
| ⁶¹Cu-NODAGA-1 | 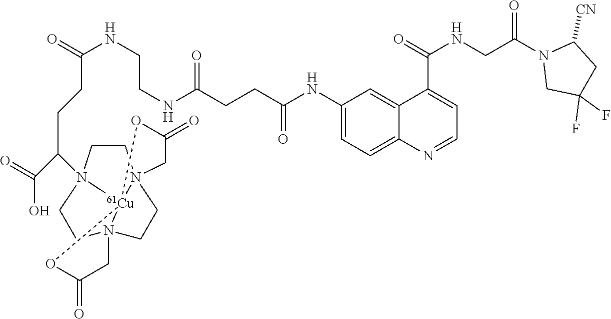 |
| ⁶¹Cu-NODAGA-2 | 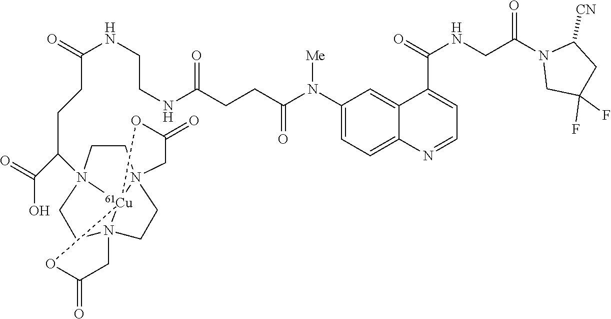 |
| ⁶¹Cu-NODAGA-3 | 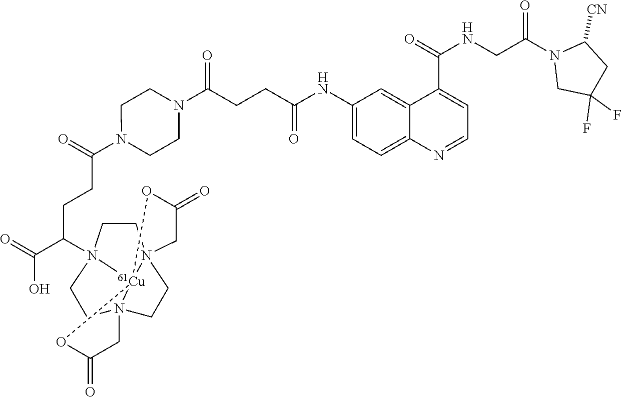 |
| ⁶¹Cu-NODAGA-4 | 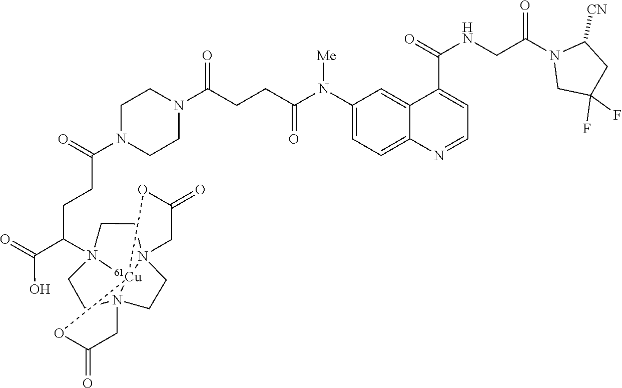 |

or is a pharmaceutically acceptable salt thereof.

The pharmaceutical compositions are useful in the methods described herein, e.g., for generating radiographic images of subjects diagnosed with or suspected of having a disorder associated with expression of FAP (e.g., a FAP-expressing cancer or tumor, fibrosis, rheumatoid arthritis, and atherosclerosis) following administration of the pharmaceutical composition, e.g., by intravenous (i.v.) administration.

In certain embodiments, the pharmaceutical composition comprises the radiotracer in an amount ≥1 μg, e.g., ≥10 μg, ≥20 μg, ≥30 μg, ≥40 μg, ≥50 μg, ≥60 μg, ≥70 μg, or ≥80 μg.

In certain embodiments, the pharmaceutical composition comprises the radiotracer in an amount from 1 μg to 100 μg, e.g., from 20 μg to 90 μg, from 20 μg to 80 μg, from 20 μg to 70 μg, from 20 μg to 60 μg, from 20 μg to 50 μg, from 20 μg to 40 μg, from 20 μg to 30 μg, from 30 μg to 100 μg, from 30 μg to 90 μg, from 30 μg to 80 μg, from 30 μg to 70 μg, from 30 μg to 60 μg, from 30 μg to 50 μg, from 30 μg to 40 μg, from 40 μg to 100 μg, from 40 μg to 90 μg, from 40 μg to 80 μg, from 40 μg to 70 μg, from 40 μg to 60 μg, from 40 μg to 50 μg, from 50 μg to 100 μg, from 50 μg to 90 μg, from 50 μg to 80 μg, from 50 μg to 70 μg, from 50 μg to 60 μg, from 60 μg to 100 μg, from 60 μg to 90 μg, from 60 μg to 80 μg, from 60 μg to 70 μg, from 70 μg to 100 μg, from 70 μg to 90 μg, from 70 μg to 80 μg, from 80 μg to 100 μg, from 80 μg to 90 μg, or from 90 μg to 100. In certain embodiments, the pharmaceutical composition comprises from 1 μg to 50 μg of the radiotracer, e.g., from 1 μg to 30 μg, from 1 μg to 10 μg, from 1 μg to 5 μg 20 μg to 100 μg, from 20 μg to 50 μg, or from 20 μg to 40 μg of the radiotracer.

In certain embodiments, the pharmaceutical composition is in the form of a liquid, e.g., a solution, such as an aqueous solution.

In certain embodiments, the pharmaceutical composition comprises an aqueous vehicle, i.e., a medium or carrier comprising at least a minimal amount of water, in which all the other components are dissolved. Exemplary aqueous vehicles include, e.g., deionized water, saline, phosphate buffer, citrate buffer, malate buffer, tartrate buffer, balanced salt solution, salts of organic acids, combinations of organic acids and salts of organic acids (e.g., tribasic sodium citrate and citric acid, malic acid and sodium malate, and potassium sodium tartrate and tartaric acid), and combinations thereof. In certain embodiments, the aqueous vehicle comprises saline solution, e.g., isotonic saline solution.

In certain embodiments, the pharmaceutical composition has a total volume of 2 to 15 mL, e.g., from 2 mL to 14 mL, from 2 mL to 12 mL, from 2 mL to 10 mL, from 2 mL to 8 mL, from 2 mL to 6 mL, from 2 mL to 4 mL, from 4 mL to 15 mL, from 4 mL to 14 mL, from 4 mL to 12 mL, from 4 mL to 10 mL, from 4 mL to 8 mL, from 4 mL to 6 mL, from 6 mL to 15 mL, from 6 mL to 14 mL, from 6 mL to 12 mL, from 6 mL to 10 mL, from 8 mL to 15 mL, from 8 mL to 14 mL, from 8 mL to 12 mL, from 8 mL to 10 mL, from 12 mL to 15 mL, or from 12 mL to 14 mL. In certain embodiments, the pharmaceutical composition has a total volume from 2 mL to 5 mL or from 8 to 12 mL.

The total volume of the pharmaceutical composition comprises one or more unit doses for administration to the subject in the methods described herein, e.g., one dose, two doses, or three or more doses. In certain embodiments, the pharmaceutical composition comprises from 2 mL to 5 mL, corresponding to one unit dose for administration. In certain embodiments, the pharmaceutical composition comprises from 8 mL to 12 mL, corresponding to two unit doses for administration.

In certain embodiments, the pharmaceutical composition comprises one or more radiolytic inhibitors. Exemplary radiolytic inhibitors include, but are not limited to, ascorbic acid, gentisic acid, citric acid, N-tert-butyl-α-phenylnitrone (PBN), polyvinylpyrrolidone (PVP), ethanol, DMSA, cysteine, vanillin, methionine, adenine, dobesilic acid, thymine, uracil, nicotinic acid, nicotinamide, salts of any of the foregoing, and combinations thereof.

In certain embodiments, the radiolytic inhibitor comprises ascorbic acid or a salt thereof. In certain embodiments, the radiolytic inhibitor comprises ascorbic acid. In certain embodiments, the radiolytic inhibitor comprises a salt of ascorbic acid, e.g., sodium ascorbate, calcium ascorbate, or potassium ascorbate.

In certain embodiments, the radiolytic inhibitor is present in the pharmaceutical composition in a concentration ≥1 mg/mL, e.g., ≥2 mg/mL, ≥3 mg/mL, or ≥5 mg/mL.

In certain embodiments, the radiolytic inhibitor is present in the pharmaceutical composition in a concentration from 1 mg/mL to 10 mg/mL, e.g., from 1 mg/mL to 7 mg/mL, from 1 mg/mL to 5 mg/mL, from 1 mg/mL to 3 mg/mL, or from 1 mg/mL to 2 mg/mL.

In certain embodiments, the radiolytic inhibitor is present in the pharmaceutical composition in a concentration ≥1 μg/mL, e.g., ≥2 μg/mL, ≥3 μg/mL, ≥5 μg/mL, ≥8 μg/mL, ≥10 μg/mL, or ≥15 μg/mL.

In certain embodiments, the radiolytic inhibitor is present in the pharmaceutical composition in a concentration from 1 μg/mL to 15 μg/mL, e.g., from 3 μg/mL to 15 μg/mL, from 3 μg/mL to 10 μg/mL, from 5 μg/mL to 10 μg/mL, from 5 μg/mL to 15 μg/mL, from 5 μg/mL to g/mL, from 8 μg/mL to 15 μg/mL, from 8 μg/mL to 10 μg/mL, or from 10 μg/mL to 15 μg/mL.

### 4.2.1. Properties

In certain embodiments, the pharmaceutical composition has an activity of ≥25 MBq, e.g., ≥30 MBq, ≥50 MBq, ≥75 MBq, ≥100 MBq, ≥150 MBq, ≥200 MBq, ≥250 MBq, ≥300 MBq, ≥350 MBq, ≥400 MBq, ≥450 MBq, ≥500 MBq, ≥750 MBq, or ≥1,000 MBq.

In certain embodiments, the pharmaceutical composition has an activity from 25 MBq to 1,500 MBq, e.g., from 25 MBq to 1,000 MBq, from 25 MBq to 750 MBq, from 25 MBq to 500 MBq, from 25 MBq to 300 MBq, from 25 MBq to 200 MBq, from 25 MBq to 100 MBq, from 50 MBq to 1,500 MBq, from 50 MBq to 1,000 MBq, from 50 MBq to 750 MBq, from 50 MBq to 500 MBq, from 50 MBq to 300 MBq, from 50 MBq to 200 MBq, or from 50 MBq to 100 MBq. In certain embodiments, the pharmaceutical composition has an activity from 100 MBq to 300 MBq. In certain embodiments, the pharmaceutical composition has an activity from 100 MBq to 200 MBq.

In certain embodiments, the pharmaceutical composition has an activity from 700 MBq to 1,500 MBq, e.g., from 700 MBq to 1,200 MBq, from 700 MBq to 1,000 MBq, from 750 MBq to 1,500 MBq, from 750 MBq to 1,200 MBq, from 750 MBq to 1,000 MBq, from 1,000 MBq to 1,500 MBq, from 1,200 MBq to 1,500 MBq, or from 1,200 MBq to 1,500 MBq.

In certain embodiments, the pharmaceutical composition has an activity from 100 MBq to 300 MBq. In certain embodiments, the pharmaceutical composition has an activity from 200 MBq to 300 MBq. In certain embodiments, the pharmaceutical composition has an activity from 100 MBq to 200 MBq. In certain embodiments, the pharmaceutical composition has an activity from 200 MBq to 800 MBq. In certain embodiments, the pharmaceutical composition has an activity from 500 MBq to 700 MBq.

In certain embodiments, the pharmaceutical composition has a radiotracer radiochemical purity of ≥90% at 12 hours after end of synthesis, e.g., ≥93%, ≥95%, ≥96%, ≥97%, ≥98%, or ≥99%.

In certain embodiments, the pharmaceutical composition has a radiotracer radiochemical purity of ≥97% at 12 hours after end of synthesis. In certain embodiments, the pharmaceutical composition has a radiotracer radiochemical purity of ≥98% at 12 hours after end of synthesis, e.g., ≥99.5%, ≥99.6%, ≥99.7%, ≥99.8≥%, or ≥99.9%.

In certain embodiments, the pharmaceutical composition has a radiotracer radiochemical purity from 90% to 99% at 12 hours after end of synthesis, e.g., from 90% to 98%, from 90% to 97%, from 90% to 95%, from 90% to 93%, from 93% to 99%, from 93% to 98%, from 93% to 97%, from 93% to 95%, from 95% to 99%, from 95% to 98%, from 95% to 97%, from 97% to 99%, or from 98% to 99%.

In certain embodiments, the pharmaceutical composition has a ⁶¹[Cu]Cu radionuclidic purity at end of synthesis of ≥95%, e.g., ≥96%, ≥97%, ≥98%, or ≥99%. In certain embodiments, the pharmaceutical composition has a ⁶¹[Cu]Cu radionuclidic purity at end of synthesis of ≥97%.

In certain embodiments, the pharmaceutical composition has a ⁶¹[Cu]Cu radionuclidic purity at end of synthesis of ≥98%, e.g., ≥99%, ≥99.5%, ≥99.6%, ≥99.7%, ≥99.8%, or ≥99.9%. In certain embodiments, the pharmaceutical composition has a ⁶¹[Cu]Cu radionuclidic purity at end of synthesis of ≥99.99%.

In certain embodiments, the pharmaceutical composition has a radiocobalt activity content at end of synthesis of ≤0.05%, ≤0.02%, or ≤0.01%, e.g., ≤0.005% or, ≤0.001%.

In certain embodiments, the pharmaceutical composition has at least one of a ⁵⁶[Co]Co specific activity or ⁵⁸[Co]Co specific activity at end of synthesis of ≤1,500 Bq/g, e.g., ≤1,200 Bq/g, ≤1,000 Bq/g, ≤800 Bq/g, ≤400 Bq/g, or ≤200 Bq/g.

In certain embodiments, the pharmaceutical composition has at least one of a ⁵⁶[Co]Co specific activity or ⁵⁸[Co]Co specific activity at end of synthesis from 200 Bq/g to 1,500 Bq/g, e.g., from 200 Bq/g to 1,200 Bq/g, from 200 Bq/g to 1,000 Bq/g, from 200 Bq/g to 800 Bq/g, from 200 to 600 Bq/g, or from 200 Bq/g to 400 Bq/g.

In certain embodiments, the pharmaceutical composition has a ⁵⁶[Co]Co specific activity of ≤100 Bq/g, e.g., ≤50 Bq/g, 25 Bq/g, ≤10 Bq/g, ≤8 Bq/g, 4 Bq/g, or 2 Bq/g.

In certain embodiments, the pharmaceutical composition has a ⁵⁶[Co]Co specific activity of from 1 Bq/g to 100 Bq/g, e.g., from 1 Bq/g to 50 Bq/g, from 1 Bq/g to 25 Bq/g, from 1 Bq/g to 10 Bq/g, from 1 Bq/g to 8 Bq/g, or from 1 Bq/g to 4 Bq/g.

In certain embodiments, the pharmaceutical composition has a ⁵⁸[Co]Co specific activity of ≤100 Bq/g, e.g., ≤50 Bq/g, 25 Bq/g, ≤10 Bq/g, ≤8 Bq/g, 4 Bq/g, or 2 Bq/g.

In certain embodiments, the pharmaceutical composition has a ⁵⁸[Co]Co specific activity of from 1 Bq/g to 100 Bq/g, e.g., from 1 Bq/g to 50 Bq/g, from 1 Bq/g to 25 Bq/g, from 1 Bq/g to 10 Bq/g, from 1 Bq/g to 8 Bq/g, or from 1 Bq/g to 4 Bq/g.

In certain embodiments, the pharmaceutical composition is characterized by a ^{110m}Ag specific activity ≤0.1 Bq/g. In certain embodiments, the pharmaceutical composition is characterized by a ^{108m}Ag specific activity ≤0.1 Bq/g. In certain embodiments, the pharmaceutical composition is characterized by a ¹⁰⁹Cd specific activity ≤0.1 Bq/g.

In certain embodiments, the pharmaceutical composition is characterized by at least two of: a ^{110m}Ag specific activity 0.1 Bq/g, a ^{108m}Ag specific activity 0.1 Bq/g, and a ¹⁰⁹Cd specific activity ≤0.1 Bq/g. In certain embodiments, the pharmaceutical composition is characterized by a ^{110m}Ag specific activity of ≤0.1 Bq/g, a ^{108m}Ag specific activity ≤0.1 Bq/g, and a ¹⁰⁹Cd specific activity ≤0.1 Bq/g.

In certain embodiments, the sum of the specific activities of the radionuclidic impurities in the pharmaceutical composition is ≤8,000 Bq/g, e.g., ≤5,000 Bq/g, ≤3,000 Bq/g, ≤1200 Bq/g, ≤1,000 Bq/g, ≤800 Bq/g, or ≤500 Bq/g.

In certain embodiments, the pharmaceutical composition comprises Al in an amount ≤1.2 ng/MBq. In certain embodiments, the pharmaceutical composition comprises Co in an amount ≤0.2 ng/MBq. In certain embodiments, the pharmaceutical composition comprises Fe in an amount ≤1.7 ng/MBq. In certain embodiments, the pharmaceutical composition comprises Pb in an amount ≤0.8 ng/MBq. In certain embodiments, the pharmaceutical composition comprises Zn in an amount ≤0.8 ng/MBq. In certain embodiments, the pharmaceutical composition comprises one or more of: an amount of Al≤1.2 ng/MBq, an amount of Co≤0.2 ng/MBq, an amount of Fe≤1.7 ng/MBq, an amount of Pb 0.8 ng/MBq, or an amount of Zn≤0.8 ng/MBq.

In certain embodiments, the pharmaceutical composition has an activity concentration ≥10 MBq/mL, e.g., ≥20 MBq/mL, ≥30 MBq/mL, ≥40 MBq/mL, ≥50 MBq/mL, ≥60 MBq/mL, ≥70 MBq/mL, ≥80 MBq/mL, or ≥90 MBq/mL.

In certain embodiments, the pharmaceutical composition has an activity concentration from 10 MBq/mL to 60 MBq/mL, e.g., from 10 MBq/mL to 50 MBq/mL, from 10 MBq/mL to 40 MBq/mL, from 10 MBq/mL to 30 MBq/mL, from 10 MBq/mL to 20 MBq/mL, from 20 MBq/mL to 60 MBq/mL, from 20 MBq/mL to 50 MBq/mL, from 20 MBq/mL to 40 MBq/mL, from 20 MBq/mL to 30 MBq/mL, from 30 MBq/mL to 60 MBq/mL, from 30 MBq/mL to 50 MBq/mL, from 30 MBq/mL to 40 MBq/mL, from 40 MBq/mL to 60 MBq/mL, from 40 MBq/mL to 50 MBq/mL, or from 50 MBq/mL to 60 MBq/mL.

In certain embodiments, the apparent molar activity of the radiotracer is ≥1 MBq/nmol, e.g., ≥10 MBq/nmol, ≥20 MBq/nmol, ≥30 MBq/nmol, or ≥50 MBq/nmol.

In certain embodiments, the apparent molar activity of the radiotracer is from 1 MBq/nmol to 50 MBq/nmol, e.g., from 1 MBq/nmol to 30 MBq/nmol, from 1 MBq/nmol to 20 MBq/nmol, from 1 MBq/nmol to 10 MBq/nmol, from 10 MBq/nmol to 50 MBq/nmol, from 10 MBq/nmol to 30 MBq/nmol, from 10 MBq/nmol to 20 MBq/nmol, from 20 MBq/nmol to 50 MBq/nmol, from 20 MBq/nmol to 40 MBq/nmol, or from 20 MBq/nmol to 30 MBq/nmol.

In certain embodiments, the molar activity of the radiotracer is ≥1 MBq/nmol, e.g., ≥10 MBq/nmol, ≥20 MBq/nmol, ≥30 MBq/nmol, or ≥50 MBq/nmol.

In certain embodiments, the molar activity of the radiotracer is from 1 MBq/nmol to 50 MBq/nmol, e.g., from 1 MBq/nmol to 30 MBq/nmol, from 1 MBq/nmol to 20 MBq/nmol, from 1 MBq/nmol to 10 MBq/nmol, from 10 MBq/nmol to 50 MBq/nmol, from 10 MBq/nmol to 30 MBq/nmol, from 10 MBq/nmol to 20 MBq/nmol, from 20 MBq/nmol to 50 MBq/nmol, from 20 MBq/nmol to 40 MBq/nmol, or from 20 MBq/nmol to 30 MBq/nmol.

In certain embodiments, the molar activity of the radiotracer is from 8 MBq/nmol to 40 MBq/nmol, e.g., from 8 MBq/nmol to 30 MBq/nmol, from 8 MBq/nmol to 20 MBq/nmol, from 20 MBq/nmol to 40 MBq/nmol, from 20 MBq/nmol to 30 MBq/nmol, or from 30 MBq/nmol to 40 MBq/nmol.

In certain embodiments, the pH of the pharmaceutical composition is from 5 to 7, e.g., from 5 to 6, 5.5 to 6.5, or from 6 to 7. In certain embodiments, the pH of the pharmaceutical composition is 5, 6, or 7+/−0.1.

### 4.2.2. Excipients

In certain embodiments, the pharmaceutical composition comprises one or more excipients.

In certain embodiments, the pharmaceutical composition comprises sodium chloride. In certain embodiments, sodium chloride is present in a concentration from 0.5 mg/mL to 50 mg/mL, e.g., from 5 mg/mL to 10 mg/mL, from 10 mg/mL to 30 mg/mL, or from 20 mg/mL to 50 mg/mL.

In certain embodiments, the pharmaceutical composition comprises ethanol. In certain embodiments, ethanol is present in an amount ≤10% v/v, e.g., ≤8% v/v, 50% v/v, or ≤3% v/v.

In certain embodiments, the pharmaceutical composition comprises ascorbic acid or a salt thereof, ethanol, and sodium chloride.

In certain embodiments, the pharmaceutical composition comprises 1-100 μg radiotracer, 5-20 mg ascorbic acid, 0.1-1 mL ethanol, and 1-10 mL isotonic saline solution.

In certain embodiments, the pharmaceutical composition comprises 20-30 μg radiotracer, 5-10 mg ascorbic acid, 0.1-0.5 mL ethanol, and 1-5 mL isotonic saline solution.

In certain embodiments, the pharmaceutical composition comprises 50-75 μg radiotracer, 15-20 mg ascorbic acid, 0.5-1 mL ethanol, and 5-10 mL isotonic saline solution.

In certain embodiments, the pharmaceutical composition comprises one or more of a metal scavenger (e.g., sodium EDTA), sodium bicarbonate, and propylene glycol.

In certain embodiments, the pharmaceutical composition comprises sodium bicarbonate. In certain embodiments, the pharmaceutical composition comprises from 0.5 to 10 mg of sodium bicarbonate, e.g., from 0.5 mg to 7.5 mg, from 0.5 mg to 5 mg, from 0.5 to 2.5 mg, from 2.5 mg to 10 mg, from 2.5 mg to 7.5 mg, from 2.5 mg to 5 mg, from 5 mg to 10 mg, from 5 mg to 7.5 mg, or from 5 mg to 10 mg. In certain embodiments, the pharmaceutical composition comprises sodium bicarbonate in an amount from 1 mg to 3 mg or 0.5 mg to 2 mg.

In certain embodiments, the pharmaceutical composition comprises a metal scavenger, e.g., EDTA or a salt thereof. In certain embodiments, the metal scavenger is sodium EDTA. In certain embodiments, the pharmaceutical composition comprises a metal scavenger (e.g., sodium EDTA) in an amount from 0.001 mg to 0.01 mg, e.g., from 0.001 mg to 0.0075 mg, from 0.001 mg to 0.005 mg, from 0.001 mg to 0.0025 mg, from 0.0025 mg to 0.01 mg, from 0.0025 mg to 0.0075, from 0.0025 mg to 0.005 mg, from 0.005 mg to 0.01 mg, from 0.005 mg to 0.0075 mg, or from 0.0075 mg to 0.01 mg. In certain embodiments, the pharmaceutical composition comprises a metal scavenger (e.g., sodium EDTA) in an amount from 0.0025 mg to 0.0075 mg or from 0.001 mg to 0.005 mg.

In certain embodiments, the pharmaceutical composition comprises propylene glycol. In certain embodiments, the pharmaceutical composition comprises propylene glycol in an amount from 5 mg to 100 mg, e.g., from 5 mg to 75 mg, from 5 mg to 50 mg, from 5 mg to 25 mg, from 25 mg to 75 mg, from 25 mg to 50 mg, from 50 mg to 75 mg, or from 50 mg to 100 mg. In certain embodiments, the pharmaceutical composition comprises propylene glycol in an amount from 10 mg to 50 mg, e.g., from 30 mg to 50 mg or from 10 mg to 20 mg.

### 4.3. Methods of Imaging

In one aspect, the present disclosure provides methods for imaging subjects suspected of having or diagnosed with a disorder associated with expression of FAP (e.g., a FAP-expressing cancer or tumor, fibrosis, rheumatoid arthritis, and atherosclerosis) comprising administering an effective amount of a pharmaceutical composition of the present disclosure and generating one or more radiographic images of the subject.

In another aspect, the present disclosure provides methods for determining a subject's response to a cancer treatment over time, e.g., a subject previously diagnosed with a FAP-expressing cancer or tumor. In certain embodiments, the method comprises:
(a) administering an effective amount of a pharmaceutical composition of the present disclosure to a subject diagnosed with a FAP-expressing cancer or tumor at an earlier time point and at a later time point;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

In certain embodiments, the subject is a mammal. In certain embodiments, the subject is a human. In certain embodiments, the subject is a human ≥18 years of age.

In certain embodiments, the subject has been diagnosed with a disorder associated with expression of FAP (e.g., a FAP-expressing cancer or tumor, fibrosis, rheumatoid arthritis, and atherosclerosis).

In certain embodiments, the subject is suspected of having, but not diagnosed with, a disorder associated with expression of FAP (e.g., a FAP-expressing cancer or tumor, fibrosis, rheumatoid arthritis, and atherosclerosis).

Exemplary cancers which can be imaged with the pharmaceutical compositions described herein include breast cancer (e.g., triple-negative breast cancer), pancreatic cancer, small intestine cancer, colon cancer, gastric cancer, rectal cancer, lung cancer (e.g., non-small cell lung cancer), head and neck cancer, ovarian cancer, hepatocellular carcinoma, epithelial cancer, esophageal cancer, hypopharynx cancer, nasopharynx cancer, larynx cancer, myeloma cells, bladder cancer, cholangiocellular carcinoma, clear cell renal carcinoma, neuroendocrine tumor, oncogenic osteomalacia, sarcoma, CUP (carcinoma of unknown primary), thymus carcinoma, desmoid tumors, glioma, astrocytoma, cervix carcinoma and prostate cancer.

Exemplary non-neoplastic diseases which can be imaged with the pharmaceutical compositions described herein include fibrosis, rheumatoid arthritis, atherosclerosis.

In certain embodiments, one or more FAP lesions are detected in the subject prior to administering the pharmaceutical composition described herein, e.g., two or more lesions or three or more lesions.

In certain embodiments, the subject is receiving cancer treatment at the time of administering the pharmaceutical composition or has received cancer treatment in the last five years prior to administering the pharmaceutical composition, e.g., at least 1 week, at least 1 month, at least 3 months, at least 6 months, at least 1 year, at least 2 years, at least 3 years, at least 4 years, or 5 years.

In certain embodiments, the cancer treatment comprises surgery, radiation therapy, ablative therapy, hormone therapy, immunotherapy, chemotherapy, drug therapy, gene therapy, cryotherapy, or a combination thereof.

In certain embodiments, the cancer treatment comprises surgery. In certain embodiments, the subject has undergone more than one surgery.

In certain embodiments, the surgery is a resection surgery, i.e., complete removal of the FAP tumor. In certain embodiments, the surgery is cytoreductive surgery, i.e., removal of a large amount of cancer/as much of the cancer as possible.

In certain embodiments, the cancer treatment comprises immunotherapy. In certain embodiments, the immunotherapy comprises administration of FAP chimeric antigen receptor (CAR) T cells.

In certain embodiments, the cancer treatment comprises drug therapy. In certain embodiments, drug therapy comprises administration of a FAP inhibitor (e.g., sibrotuzumab).

In certain embodiments, the cancer treatment comprises chemotherapy. In certain embodiments, chemotherapy comprises administration of an alkylating agent. In certain embodiments, chemotherapy comprises administration of platinum agents.

In certain embodiments, the pharmaceutical composition is administered intravenously. In certain embodiments, administration occurs as a single bolus infusion. In certain embodiments, administration occurs over two or more separate infusions.

In certain embodiments, the pharmaceutical composition is administered over a period of time of at least 5 seconds, e.g., at least 10 seconds, at least 15 seconds, at least 20 seconds, at least 30 seconds, at least 1 minute, at least 5 minutes, or at least 10 minutes.

In certain embodiments, the pharmaceutical composition is administered over a period of time from 5 seconds to 10 minutes, e.g., from 5 seconds to 5 minutes, from 5 seconds to 1 minute, from 5 seconds to 30 seconds, from 5 seconds to 20 seconds, from 10 seconds to 10 minutes, from 10 seconds to 5 minutes, from 10 seconds to 1 minute, from 10 seconds to 30 seconds, from 30 seconds to 10 minutes, from 30 seconds to 5 minutes, from 30 seconds to 1 minute, from 1 minute to 10 minutes, from 1 minute to 5 minutes, or from 5 minutes to 10 minutes.

In certain embodiments, the effective dose per MBq of administered pharmaceutical composition is ≤20 μSv/MBq, e.g., ≤15 μSv/MBq, ≤10 μSv/MBq, ≤7.5 μSv/MBq, ≤5 μSv/MBq, or ≤3 μSv/MBq.

In certain embodiments, the effective dose per MBq of administered pharmaceutical composition is from 3 μSv/MBq to 20 μSv/MBq, e.g., from 3 μSv/MBq to 15 μSv/MBq, from 3 μSv/MBq to 10 μSv/MBq, from 3 μSv/MBq to 7.5 μSv/MBq, from 3 μSv/MBq to 5 μSv/MBq, from 5 μSv/MBq to 20 μSv/MBq, from 5 μSv/MBq to 15 μSv/MBq, from 5 μSv/MBq to 10 μSv/MBq, from 5 μSv/MBq to 7.5 μSv/MBq, from 7.5 μSv/MBq to 20 μSv/MBq, from 7.5 μSv/MBq to 15 μSv/MBq, from 7.5 μSv/MBq to 10 μSv/MBq, from 10 μSv/MBq to 20 μSv/MBq, from 10 μSv/MBq to 15 μSv/MBq, or from 15 μSv/MBq to 20 μSv/MBq.

In certain embodiments, the effective dose per MBq of administered pharmaceutical composition is from 5 μSv/MBq to 10 μSv/MBq, e.g., from 5 μSv/MBq to 9 μSv/MBq, from 5 μSv/MBq to 8 μSv/MBq, from 5 μSv/MBq to 7 μSv/MBq, or from 5 μSv/MBq to 6 μSv/MBq.

The methods of the present disclosure include a step of generating one or more radiographic images of the subject. The images can be of a region, body part, or organ of the subject. In certain embodiments, the one or more images are generated of the skull to mid-thigh of the subject. In certain embodiments, the one or more images are generated of the head and neck of the subject. In certain embodiments, the one or more images are generated of the whole body of the subject. Exemplary organs include, but are not limited to, breasts, pancreas, small intestine, colon, rectum, lung, bread, ovaries, liver, skin, esophagus, pharynx, nasopharynx, larynx, bladder, kidney, thymus, bones, joints, cervix, and prostate.

The radiographic images can be generated using positron emission tomography (PET), PET-computer tomography (PET-CT), or single-photon emission computerized tomography (SPECT). In certain embodiments, the one or more radiographic images are generated using PET. In certain embodiments, the one or more radiographic images are generated using PET-CT. In certain embodiments, the one or more radiographic images are generated using SPECT.

In certain embodiments, the one or more radiographic images of the subject are generated dynamically, i.e., concurrently with administration of the pharmaceutical composition.

In certain embodiments, the one or more radiographic images are generated after administration of the pharmaceutical composition. In certain embodiments, the one or more radiographic images are generated at least 30 minutes after administration of the pharmaceutical composition, e.g., at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, or at least 24 hours after administration of the pharmaceutical composition.

In certain embodiments, the one or more radiographic images are generated from 30 minutes to 24 hours after administration of the pharmaceutical composition, e.g., from 30 minutes to 18 hours, from 30 minutes to 10 hours, from 30 minutes to 5 hours, from 30 minutes to 3 hours, from 30 minutes to 1 hour, from 1 hour to 24 hours, from 1 hour to 18 hours, from 1 hour to 10 hours, from 1 hour to 5 hours, from 1 hour to 3 hours, from 3 hours to 24 hours, from 3 hours to 18 hours, from 3 hours to 10 hours, from 3 hours to 5 hours, from 5 hours to 24 hours, from 5 hours to 18 hours, from 5 hours to 10 hours, from 10 hours to 24 hours, from 10 hours to 18 hours, or from 18 hours to 24 hours after administration of the pharmaceutical composition.

In certain embodiments, the one or more radiographic images are generated 1 hour after administration of the pharmaceutical composition. In certain embodiments, the one or more radiographic images are generated 3 hours after administration of the pharmaceutical composition. In certain embodiments, the one or more radiographic images are generated 18 hours after administration of the pharmaceutical composition. In certain embodiments, the one or more radiographic images are generated 24 hours after administration of the pharmaceutical composition.

In certain embodiments, the method further comprises determining one or more of the following from the radiographic images: maximum standardized uptake volume (SUVmax), tumor detection rate (TDR), differential tumor detection rate (DDR), signal-to-noise ratio (SNR=SUVmax in tumor volume of interest (VOI)), tumor-to-background ratio(s) (TBR), sensitivity, and positivity predictive value (PPV).

In certain embodiments, following generating one or more images, localization (absolute) and amount of localization of the radionuclide in the one or more radiographic images of the subject can be determined, e.g., by medical personnel.

In embodiments where the subject is suspected of having or diagnosed with a FAP-expressing cancer, one or more of the following can occur based on the localization of the radionuclide: staging or restaging the subject's cancer, determining the subject's cancer treatment protocol, and determining the subject's response to cancer treatment.

In certain embodiments, reading of the one or more radiographic images generated by the methods described herein enables staging the subject's cancer. In certain embodiments, reading of the one or more radiographic images generated by the methods described herein enables restaging the subject's cancer. A number of staging methods are known in the art, e.g., the “TNM system,”, where “T” is the primary tumor (T category), “N” is lymph nodes (N category” and “M” is metastasis (M category). In certain embodiments, a cancer's grade is included in the stage. Once the values for T, N, and M (and any other factors that affect stage) have been determined, they are combined to assign an overall stage. For most cancers, the stage is a Roman numeral from I (1) to IV (4). Stage I cancers are less advanced and often have a better prognosis (outlook). Higher stage cancers typically have spread farther (or have other concerning features) and might require more intense (or different kinds of) treatment. Sometimes stages are subdivided as well, using capital letters (for example, stage III might be subdivided into stages IIIA and IIIB). Some cancers also have a stage 0, which is often called carcinoma in situ. This means the cancer is still only in the layer of cells where it first started, and it has not spread any farther.

In certain embodiments, reading of the one or more radiographic images generated by the methods described herein enables determination of the subject's cancer treatment protocol, e.g., surgery, radiation therapy, ablative therapy, hormone therapy, gene therapy, cryotherapy, immunotherapy, chemotherapy, or a combination thereof.

In certain embodiments, reading of the one or more radiographic images generated by the methods described herein enables determination of the subject's response to cancer treatment, e.g., responding to treatment or not responding to treatment. In such embodiments, the pharmaceutical composition is administered to the subject at an earlier time point and at a later time point, and one or more images are generated at both the earlier time point and the later time point. The images generated at the earlier time point and later time point are compared. Subjects responding to treatment have decreased radiotracer localization at the later imaging time point compared to the earlier imaging timepoint.

In certain embodiments, the earlier time point is before the subject begins cancer treatment. In certain embodiments, the earlier time point is at least 1 day before the subject begins cancer treatment, e.g., at least 1 week, at least 2 weeks, at least 3 weeks, or at least 1 month before the subject begins cancer treatment.

In certain embodiments, the earlier time point is after the subject begins cancer treatment. In certain embodiments, the earlier time point is at least 1 week after the subject begins cancer treatment, e.g., at least 2 weeks, at least 1 month, at least 2 months, at least 3 months, at least 6 months, at least 9 months, or at least 12 months after the subject begins cancer treatment.

In certain embodiments, the later time point is at least 1 day after the earlier time point, e.g., at least 2 days, at least 3 days, at least 4 days, at least 5 days, at least 6 days, at least 1 week, at least 2 weeks, at least 3 weeks, at least 4 weeks, at least 2 months, at least 3 months, at least 6 months, at least 9 months, or at least 1 year after the earlier time point.

In certain embodiments, the later time point is after the subject finishes cancer treatment. In certain embodiments, the later time point is at least 1 week after the subject finishes cancer treatment, e.g., at least 2 weeks, at least 1 month, at least 2 months, at least 3 months, at least 6 months, at least 9 months, or at least 12 months after the subject finishes cancer treatment.

In certain embodiments, the earlier time point is before the subject begins cancer treatment and the later time point is after the subject finishes cancer treatment (i.e., to determine the effectiveness of a previous treatment). In certain embodiments, the earlier time point is before the subject begins cancer treatment and the later time point is after the subject begins cancer treatment (i.e., to determine the effectiveness of the ongoing treatment).

### 5. EXAMPLES

### 5.1. Example 1: [⁶¹Cu]CuCl₂ Production

The production of the radionuclide was performed by irradiating a solid target of highly pure ⁶¹Ni (purity ≥99.42%) plated on a niobium coin backing with a proton beam.

Due to the relatively short half-lives (t_{1/2} ⁶⁸Ga=68 min; ¹⁸F=110 min) and physical properties of the radionuclides, the key challenges in the PET tracer industry remain a) the imaging quality, b) reliability of supply and distribution of the radiopharmaceutical at low cost and c) low radiation burden to the patient. The distinctive advantage of using ⁶¹Cu as a positron emitter, e.g., in a PET tracer, will not only ensure a) good imaging quality due to its physical properties (low mean positron energy) but also the possibility of delayed imaging, expected to improve the diagnostic sensitivity due to the washout of radioactivity from the background, thus improved image contrast, b) a large distribution radius due to its relatively long half-life (t_{1/2} ⁶¹Cu=205.5 min) while c) still keeping the radiation burden to the patient at a minimum. Provided herewith is an enabling description of new processes to produce highly pure ⁶¹Cu, in the form of [⁶¹Cu]CuCl₂, to be used in radiopharmaceutical applications, e.g., as a positron emitter in a PET tracer, in high activity concentration and volumes.

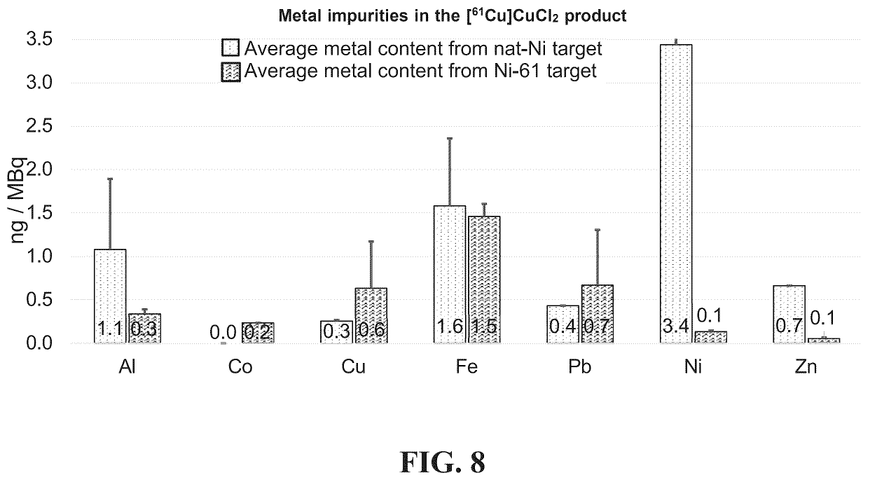

Trace metals and cold copper compete with ⁶¹Cu to bind a chelator (for example, NODAGA) in this order: cold Cu(II) (i.e., stable isotopes)>Zn(II)>Fe(III)>Sn(IV)>Ti(IV)>Al(III.). The competition from these trace metals and cold copper decreases the tracer's radiolabeling yield and radiochemical purity significantly. Frequent sources of trace metals are the raw nickel metal powder itself, especially isotopically enriched nickel, reagents, and any metals in instruments used, such as iron. The purification process (ion-exchange columns) removes much of the trace metals except for cold (of particular relevance are stable isotopes ⁶⁹Cu and ⁶⁵Cu), which passes through into the product fraction by being the same element as the desired ⁶¹Cu. One way of preventing cold copper contamination and the associated reduction in chemical purity is to pass the dissolved nickel raw material (stable isotopes) through the process and separate the cold copper from the nickel before plating (see FIG. 8 for ICP-MS analysis). Table 1 displays the chemical purity of the [⁶¹Cu]CuCl₂ by either bombardment of ^{nat}Ni or ⁶¹Ni on a niobium backing and the resulting impurity profile.

TABLE 1
Chemical Purity of ⁶¹Cu transmuted from ^{nat}Ni vs. ⁶¹Ni.

ng/MBq

| | Cu-61 | Cu-61 |
|---|---|---|
| | from | from |
| | nat-Ni | Ni-61 |
| Aluminum (Al) | 1.1 | 0.3 |
| Cobalt (Co) | N/D | 0.2 |
| Copper (Cu) | 0.3 | 0.6 |
| Iron (Fe) | 1.6 | 1.5 |
| Lead (Pb) | 0.4 | 0.7 |
| Nickel (Ni) | 3.4 | 0.1 |
| Zinc (Zn) | 0.7 | 0.1 |

### 5.1.1. Preparation of Plating Solution

### 5.1.1.1 Preparation of Buffer Solution

Ammonium Chloride (4.6 g, Aldrich: 326372, Trace Select) was weighed into a clean (no metal) Falcon Tube (50 mL), and the previously cleaned magnetic stirring bar was added. 6 mL of Trace Select water (Honeywell 95305) was added in one aliquot to flush walls of the Falcon in case any salt sticks to the Falcon tube walls. 1 mL of ammonium hydroxide 28% (Sigma 338818) was added with a 1000 μL pipette with a respective pipette tip, 8× times. The lid of the Falcon was closed, and the Falcon is, in turns, vortexed (1-2 minutes) (immersion in an ultra-sonic bath was a possible alternative for 1-2 minutes) and shaken, until all salt was dissolved. The Falcon tube can also be warmed (e.g., by rolling between hands) to improve solubility, temperature (e.g., around 23° C., preferably between 23-25° C.). After complete dissolution of the salt, the pH acceptance criteria, pH range 9.28-9.62, was verified by pH measurement of the solution at RT, e.g., with and electronic pH meter. The Falcon tube was closed with parafilm and stored at room temperature. Prior to use, any solid salt formation was redissolved.

### 5.1.1.2 Preparation of Nickel Nitrate Plating Solution

A 50 mL glass beaker was washed with nitric acid (Trace Select) followed by water (Trace Select). In a fume hood, the beaker was dried by placing it on a heating plate set to 150° C. To the beaker was added 210 μg of natural (isotopic distribution) nickel (powder, Sigma-Aldrich <50 μm, 99.7% trace metals basis, essentially free from any impurities, except iron. The copper impurity amounts to <0.3 ppm.) were weighed into the beaker and 4 mL of 65% nitric acid were added using a pipette. The beaker was placed back on the active heating plate and the stirring was set to 300 rpm. Ensure the ventilation of the fume hood was functioning properly (evolution of NO₂). During the dissolution, the solution turned green. The solution was reduced by evaporation to a volume of & 600 μL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. Buffer solution (4 mL), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

The following are example lots of ⁶⁰Ni and ⁶¹Ni (certificate as provided by Isoflex, USA, March 2018):

TABLE 2

| Isotope | ⁶¹Ni | | | | | | | | | | |
|---|---|---|---|---|---|---|---|---|---|---|---|
| Enrichment | 86.20% | | | | | | | | | | |
| Form | Metal | | | | | | | | | | |
| | ingot/powder | | | | | | | | | | |
| Certificate | 6275 | | | | | | | | | | |
| Isotopic | | | | | | | | | | | |
| distribution | Isotope | Ni-58 | Ni-60 | Ni-61 | Ni-62 | Ni-64 | | | | | |
| | Content (%) | 1.17 | 0.8 | 86.2 | 11.7 | 0.14 | | | | | |
| Chemical | | | | | | | | | | | |
| admixtures | Element | Al | Bi | Ca | Cd | Co | Cr | Cu | Fe | K | μg |
| | Content | 10 | <10 | 20 | 10 | <10 | <10 | 20 | 40 | <10 | <50 |
| | (ppm) | | | | | | | | | | |
| | Element | Mo | Mn | Na | Pb | Si | Sn | Zn | | | |
| | Content | <8 | <50 | <10 | <10 | 20 | 30 | 50 | | | |
| | (ppm) | | | | | | | | | | |

TABLE 3

| Isotope | ⁶¹Ni | | | | | | | | | | |
|---|---|---|---|---|---|---|---|---|---|---|---|
| Enrichment | 99.39% | | | | | | | | | | |
| Form | Metal | | | | | | | | | | |
| | powder | | | | | | | | | | |
| Certificate | TBD/not | | | | | | | | | | |
| | specified | | | | | | | | | | |
| Isotopic | | | | | | | | | | | |
| distribution | Isotope | Ni-58 | Ni-60 | Ni-61 | Ni-62 | Ni-64 | | | | | |
| | Content | 0.01 | 0.29 | 99.39 | 0.29 | 0.02 | | | | | |
| | (%) | | | | | | | | | | |
| Chemical | | | | | | | | | | | |
| admixtures | Element | Al | Co | Cr | Cu | Fe | μg | Mn | Pb | Si | Ti |
| | Content | 12 | <10 | <10 | 14 | <10 | <10 | <10 | <10 | <10 | <10 |
| | (ppm) | | | | | | | | | | |
| | Element | Zn | C | S | | | | | | | |
| | Content | <10 | 157 | <10 | | | | | | | |
| | (ppm) | | | | | | | | | | |

TABLE 4

| Isotope | ⁶⁰Ni | | | | | | | | | | |
|---|---|---|---|---|---|---|---|---|---|---|---|
| Enrichment | 99.31% | | | | | | | | | | |
| Form | metal | | | | | | | | | | |
| | powder | | | | | | | | | | |
| Certificate | TBD/not | | | | | | | | | | |
| | specified | | | | | | | | | | |
| Isotopic | | | | | | | | | | | |
| distribution | Isotope | Ni-58 | Ni-60 | Ni-61 | Ni-62 | Ni-64 | | | | | |
| | Content | 0.21 | 99.31 | 0.46 | 0.015 | 0.005 | | | | | |
| | (%) | | | | | | | | | | |
| Chemical | | | | | | | | | | | |
| admixtures | Element | Al | Co | Cr | Cu | Fe | μg | Mn | Pb | Si | Ti |
| | Content | <10 | 70 | 20 | 25 | <10 | <10 | <10 | <10 | 15 | <10 |
| | (ppm) | | | | | | | | | | |
| | Element | Zn | C | S | P | | | | | | |
| | Content | 15 | 114 | 20 | 30 | | | | | | |
| | (ppm) | | | | | | | | | | |

The samples of natural nickel from Sigma-Aldrich were essentially free from any impurities, except iron. The copper impurity amounts to <0.3 ppm. Additional suitable sources of natural Ni include:
Nickel powder, <50 μm, 99.7% trace metals basis
Nickel rod, diam. 6.35 mm, =99.99% trace metals basis
Nickel foil, thickness 0.5 mm, 99.98% trace metals

### 5.1.2. Electroplating the Backing Surface

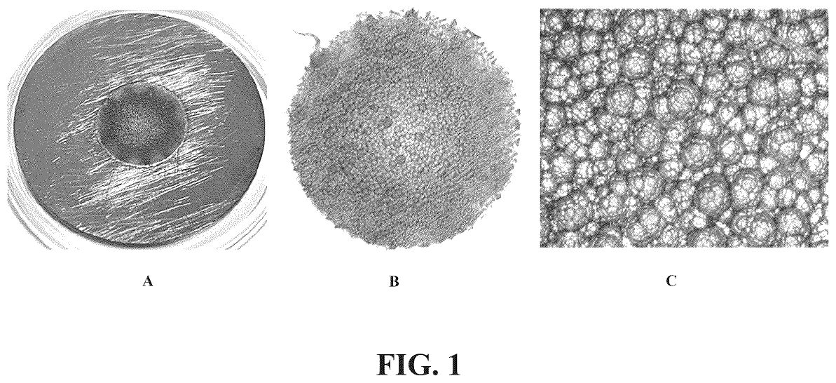

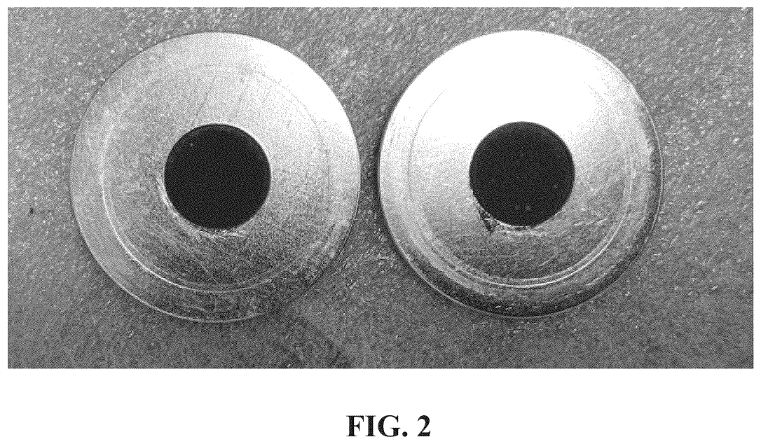

A disc shaped niobium backing was obtained from high purity Nb as described herein and (28 mm×1.0 mm) was cleaned with ethanol (high-purity) and inserted in a Comecer Electroplating Unit V21204. A platinum wire anode was positioned so that the distance relative to the coin surface was between about 1 and 3 mm, adjusted by a polymer spacer. The coin mass was determined to be 5.25 grams. Niobium backing (22 mm×1.0 mm weighs 3.3 g). The plating solution was charged to the electrolyte container and attached to the apparatus. The voltage was set to 4.5V. The current reading after 5 min stabilization was 180 μA. The duty cycle for pump was set to 45%. The plating liquid turned from blue to transparent, slow decrease of current to 160 μA was observed over the period of 120 minutes. The plating process was stopped. The coin was taken out of the electrolytic cell and its weight was measured. The coin also underwent microscopic evaluation, FIG. 1 and FIG. 2 using a DINOLite digital microscope to observe the crystal structure and homogeneity of the surface. The coin (FIG. 2) was stored in a metal-free Falcon tube under a nitrogen atmosphere.

### 5.1.3. Results of the Electroplating

Upon completion of electroplating, the coin underwent a microscopic evaluation using a DINOLite digital microscope to observe the crystal structure and homogeneity of the surface. As can be seen in FIG. 1 (panels A-C), a homogenous target coating having durable adhesion was obtained, see also FIG. 2.

### 5.1.4. General Guidelines for High-Purity [⁶¹Cu]Cl₂ Production

The purpose of this example was to enable the bulk production of ⁶¹Cu from the deuteron irradiation of natural nickel and/or enriched ⁶⁰Ni. This effort was a proof of concept, and, therefore, there were no benchmarked specifications for ⁶¹Cu. However, we optimized target performance, target geometry/material use, irradiation parameters, and chemical processing methods to produce [⁶¹Cu]CuCl₂ following enriched ⁶⁰Ni irradiation, or, scaled accordingly for ^{nat}Ni irradiation. There were no pharmacopoeia specifications for radio-copper explicitly, however, test QC methods include assessment of radionuclidic purity and molar activity (to demonstrate usability of the extracted [⁶¹Cu]CuCl₂).

This example considers use of two different types of targets, natural nickel (^{nat}Ni) targets and highly enriched Nickel-60 (⁶⁰Ni) targets both of which were suitable for deuteron bombardment. However, ^{nat}Ni was cheap and available in high-purity while ⁶⁰Ni was still costly and required efficiency measures. If even higher yields were desired, target preparation efforts may be directly translated into the proton-based ⁶¹Ni(p,n)⁶¹Cu route, however, given the cost of enriched ⁶¹Ni (c.a. $25 USD/μg), such an approach imposes the need for target recycling.

The set of guidelines below enable all types of targets in the production of ⁶¹Cu, including the production of high-purity [⁶¹Cu]CuCl₂ from the Nb coins with a Zn or Ni (any isotopic enrichment) coating electroplated thereon as provided herein. Specific details are also provided for deuteron, and proton irradiations, respectively. This protocol was followed to generate the ⁶¹Cu-compositions evaluated in the following examples.

| Target Backing | Flat coin - disc-shaped. The dimensions of the target backing form are: | | |
|---|---|---|---|
| Geometry | Coin backing: | | |
| | Diameter Ø = 20 − 30 ± 0.1 mm | | |
| | Thickness H = 1.5 mm | | |
| | Target Ni layer or coating | | |
| | a. Diameter 13 mm (deuteron) or 10 mm (proton) | | |
| | b. Mass 70-100 mg, e.g., around 100 ± 40 mg (deuteron) or | | |
| | around 50 ± 20 mg (proton) | | |
| | c. Thickness (H) full density (d = 8.9) | | |
| | i. Hₘᵢₙ = 0.1 mm; Hₘₐₓ = 0.14 mm corresponding to | | |
| | 70-100 mg deposited | | |
| | Tolerances/finishes unless otherwise stated are as follows: | | |
| | Surface finish: Ra 1.6 | | |
| | General tolerance: ISO 2768-m | | |
| | Sharp edges and corners according to ISO 13 715 | | |
| Target Backing | Optional - Surface treated with abrasion by pink corundum | | |
| Surface | grindstone - free of impurities | | |
| Target Backing | Niobium foil, 99.8% (metals basis), 1.0 mm (0.04 in) thick, annealed, | | |
| Material | Stock No.: 10257 | | |
| | Lot No.: C15P07 | | |
| | Element | ppm | |
| | Carbon | 24 | |
| | Hydrogen | 1 | |
| | Molybdenum | 2 | |
| | Nickel | 4 | |
| | Silicon | 1 | |
| | Titanium | 2 | |
| | Zirconium | 3 | |
| | Iron | 1 | |
| | Hafnium | 2 | |
| | Nitrogen | 14 | |
| | Oxygen | 56 | |
| | Tantalum | 785 | |
| | Tungsten | 4 | |
| Target Backing | Niobium 99.9% typical certificate of analysis results, Goodfellows | | |
| Material | Product nr. 931-627-20 | | |
| | Element | ppm | |
| | B | <10 | ppm |
| | Ni | <5 | ppm |
| | O | 100 | ppm |
| | Si | 100 | ppm |
| | Zr | <10 | ppm |
| | Ta | 500 | ppm |
| | H | <10 | ppm |
| | W | <100 | ppm |
| | C | 25 | ppm |
| | N | 20 | ppm |
| | Fe | 30 | ppm |
| | Cu | <5 | ppm |
| | Mo | 10 | ppm |
| | Ti | <10 | ppm |

| Transfer system | As the target can be automatically transferred to/from the cyclotron by | |
|---|---|---|
| compatibility | means of a pneumatic target transfer system, it was critical that the | |
| | deposited Ni was robust to direct air flow and abrupt mechanical | |
| | movements. | |
| | In certain embodiments, the target coating remains adhered to the backing | |
| | during pneumatic transfer both to and from the cyclotron. Such a pneumatic | |
| | system was typically fed by a compressed air connection of ~6-7 bar, and at | |
| | minimum, 360 SLPM flow. Such a system was “push-push”, and therefore, | |
| | compressed air was typically blown on both the front and rear sides of the | |
| | coin, respectively, depending on the direction of transfer. The coin will also | |
| | come to an abrupt stop as it reaches the target station or hot cell. | |
| | In certain embodiments, suitable tests that indicate target durability include | |
| | the following, whereby the total mass loss for all tests combined should be | |
| | negligible (e.g. <1 μg): Visual inspection, gentle knocking/tapping on a | |
| | countertop on top of white paper to check for loosening of target coating | |
| | grains, gently rubbing an acid-washed Teflon spatula against the deposited | |
| | target coating and checking for loosening of target coating grains, and/or | |
| | placing and gently pressing down on a piece of Scotch tape against the | |
| | target coating. | |
| | If there was access to the cyclotron apparatus, it was recommended to | |
| | transfer the coin back/forth multiple times and ensure target coating | |
| | stability (i.e., no mass loss). Such a test may be performed with a degrader | |
| | in place. | |
| Method of | Electrodeposition from bath with a significantly high pH (e.g., 9.9-10.8) | |
| Production | | |
| Target Metal | To withstand the deposited beam power, the target metal was preferably | |
| Form | metallic nickel (not, e.g., nickel oxide). | |
| | Depending on the means of target preparation (e.g. electroplating), the raw | |
| | nickel starting material need not necessarily be metallic. However, methods | |
| | used for preparing ^{nat}Ni targets should ultimately be directly translatable to | |
| | preparation of ⁶⁰Ni or ⁶¹Ni targets. At present, it was understood that | |
| | enriched Ni was typically in the form of a salt. | |
| Target Additives | The use of binders must not necessary be avoided if they are absent of the | |
| | final metallic coin and if an assessment on a case-by-case basis to | |
| | understand potential impact to product quality has been done (e.g. ICP-MS | |
| | on the binder material. | |
| | Any reagents used for target preparation (e.g. electroplating reagents) must | |
| | be of the highest quality, in particular, with regards to trace metals. | |
| Metal Content | Preferably, the highest grades of reagents should be used, to avoid trace | |
| | metals contamination of the target coating, as more than a tenth of a | |
| | microgram per 100 μg of target metal (that is, 1 ppm of the target metal) is | |
| | already a significant contamination that may render the coin unusable for | |
| | production of high-purity radionuclides. In the case of the production of | |
| | radiocopper it is not accepted to add more than 0.1 ppm of cold Cu as this | |
| | would reduce the purity of the prepared radionuclide composition. | |
| | Preferably, max level of impurities allowed to be added by the process to | |
| | the initial nickel: | |
| | Copper (Cu): 0.1 ppm | |
| | High affinity metals (Ga, Lu, Pb, Y): 0.1 ppm | |
| | Zinc and cobalt (Zn, Co): 0.3 ppm | |
| | Transition and other metals (Cd, Cr, Al, Mn, Mo, Sn, Ti, V . . . ): 1 | |
| | ppm on a case by case | |
| | Iron (Fe): 10 ppm | |
| | Family I and II (K, Ba, μg, Be . . . ): 1000 ppm | |
| | The metal coins were analyzed on a batch per batch basis by dissolution in | |
| | nitric acid to assess the metal contamination within the coin that were not | |
| | found in the starting nickel metal and thus originate from the process. | |
| | The amount suggested above were a good, albeit not strict, guide since | |
| | chemical purification following irradiation will, in turn, further remove | |
| | some of these impurities. The ultimate specification on this front will | |
| | therefore be an iterative process as the Cu/Ni separation chemistry is | |
| | refined. However, the process shall not significantly add impurities that | |
| | were not in the originating pure nickel material. | |
| | It is preferable that cold Cu should be minimized in the deposited Ni since | |
| | this will follow the chemistry of any ⁶¹Cu and cannot be separated | |
| | post-irradiation. Any such cold Cu will directly compete with ⁶¹Cu | |
| | during radiolabeling of the pharmaceutical. Methods of removing Cu | |
| | from the dissolved target metal are well known. | |
| Density of Target | To withstand the deposited beam power, the Ni target should preferably be | |
| | of reasonably high volumetric density (e.g. approximately ≥90% or, ≥8.0 | |
| | g/cm³). | |
| Power Rating | The power rating for the target, including the combined deposited Ni and | |
| | plate should preferably | |
| | be: ≥420 W (deuterons) ≥820 W (protons) | |
| Loading Mass of | The loading mass vs. the deposited mass of Ni (i.e. deposition efficiency) | |
| Target | relates not to technical specifications, but rather, to cost. In the case of ^{nat}Ni | |
| | deposition, loading efficiency will not have a significant impact on the cost | |
| | of ⁶¹Cu. However, losses should be minimized in considering the translation | |
| | to enriched ^{6x}Ni. For ⁶⁰Ni, losses should be maintained below ~10%, and | |
| | for ⁶¹Ni, below ~1%. Some techniques such as magnetron sputtering are | |
| | thus not possible for enriched nickel but are satisfactory for ^{nat}Ni. | |
| Mass/thickness | For deuterons (i.e. ^{nat}Ni or ⁶⁰Ni), the thickness should be appropriate for | |
| of Nickel | stopping the deuterons, with a maximum 10% variability in material | |
| | deposition. Such thicknesses equate | |
| | to: ≥100 μm (assuming 100% density) ≥70 mg | |
| | or ≥89 mg/cm² (assuming 10 mm diameter) | |
| | For protons (i.e. ⁶¹Ni), one may wish to selectively limit the deposited | |
| | material to optimize the balance between material cost, yield, and backing | |
| | material activation. With a maximum 10% variability in material | |
| | deposition, four examples are noted below. | |
| | ⁶¹Ni Scenario #1 (11→9 MeV) | |
| | 78 μm (assuming 100% density) | |
| | 55 mg or 69 mg/cm² (assuming 10 mm diameter well) | |
| | ⁶¹Ni Scenario #1 (12→8 MeV) | |
| | 155 μm (assuming 100% density) | |
| | 108 mg or 138 mg/cm² (assuming 10 mm diameter well) | |
| | ⁶¹Ni Scenario #1 (13→7 MeV) | |
| | 233 μm (assuming 100% density) | |
| | 163 mg or 208 mg/cm² (assuming 10 mm diameter well) | |
| | ⁶¹Ni Scenario #1 (13→4 MeV) | |
| | 309 μm (assuming 100% density) | |
| | 216 mg or 275 mg/cm² (assuming 10 mm diameter well) | |
| Isotopic | The ^{6x}Cu radioisotopes which will be coproduced during production of ⁶¹Cu | |
| enrichment | (t ½ = 3.339 h) include: | |
| | ⁵⁷Cu (t ½ = 0.196 s) | ⁵⁸Cu (t ½ = 3.204 s) |
| | ⁵⁹Cu (t ½ = 81.5 s) | ⁶⁰Cu (t ½ = 23.7 m) |
| | ⁶²Cu (t ½ = 9.673 m) | ⁶⁴Cu (t ½ = 12.701 h) |

| | |
|---|---|
| | From a practical handling point of view, all but ⁶⁰Cu and ⁶⁴Cu are likely to |
| | have decayed prior to use. Only ⁶⁴Cu will have any impact on the possible |
| | shelf-life of ⁶¹Cu. |
| | In addition to the production of Cu radioisotopes, other radionuclides (e.g. |
| | Co and Ni) will also be produced, the ratio of which will depend on the |
| | isotopic composition, and whether undergoing deuteron or proton |
| | irradiation. As these byproducts are chemically different from copper, such |
| | radionuclides may be removed during ⁶¹Cu purification/processing. For |
| | example, The Cu-61 was purified from metal and radiometal impurities via |
| | a GE Healthcare FASTlab 2 module through a tributyl phosphate resin |
| | cartridge and a tertiary-amine-based weak ionic exchange resin containing |
| | long-chained alcohols. |
| Any other | Niobium is preferred over silver for its better resistance to corrosion, its low |
| requirements | amount of activation on irradiation and for its high melting temperature that |
| | permits the deposit of nickel by other processes such as melting or heat |
| | sintering. However, silver possesses a higher thermal conductivity and may |
| | be suitable for certain embodiments. |
| | For target backing manufacture, the following sheet of niobium is suitable |
| | for laser cutting: |
| | http://www.Goodfellow.com |
| | NB000400 Niobium Foil, Size: 150 × 150 mm Thickness: 1.5 mm, |
| | Purity: 99.9%, Temper: Annealed, Quality: LT |
| | From one sheet up to 25 target backings can be manufactured. |

### 5.1.5. Purification and Characterization of [⁶¹Cu]CuCl₂ and Waste Streams

The solid target irradiated material was dissolved in a total volume of 7 mL of 6 M HCl with the addition of 30% hydrogen peroxide via a dissolution chamber. Separation and purification was accomplished using a cassette-based FASTlab platform using a TBP (tributylphosphate-based) resin (1 mL) (particle size 50-100 μm; pre-packed, Triskem) then a weakly basic (tertiary amine; TK201) resin (2 mL) (particle size 50-100 μm; pre-packed, Triskem), each of which were pre-conditioned with H₂O (7 mL) and HCl (10M, 7 mL). The cassette reagent vials were prepared using concentrated HCl (Optima Grade, Fischer Scientific), NaCl (ACS, Fischer Scientific) and milli-Q water (Millipore system, 18 MΩ-cm resistivity). 6M HCl (2×4.2 mL), 5M NaCl in 0.05 M HCl (4.2 mL). The subsequent ⁶¹Cu was then purified with two subsequent ion exchange resins in a FASTlab synthesis unit.
1) The acid-adjusted dissolution solution (approx. 7 mL) was loaded over both columns in series and directed into a “Ni collection fraction”. The TBP resin acted as a guard column as it quantitatively retained Fe³⁺ ions, while the Cu²⁺ and Co²⁺ complexes were quantitatively retained on the tertiary amine (TK201) resin.
2) Both columns were washed with 6M HCl (4 mL) to maximize Ni recovery for future recycling.
3) The TK201 column was washed with 4.5M HCl (5.5 mL) to elute the majority of cobalt salts.
4) The TK201 column was washed with 5M NaCl in 0.05M HCl (4 mL) to decrease residual acid on the resin and further remove any residual cobalt salts.
5) The TK201 column was washed with of 0.05M HCl (3 mL) to quantitatively elute the [⁶¹Cu]CuCl₂.

The resulting [⁶¹Cu]CuCl₂ solution of the plated material has an average activity of 1.0-4.5 GBq. This activity was measured using a dose calibrator from Comecer and its radionuclidic purity by a gamma spectrometer at PSI in Switzerland.

Gamma spectrometry measurements were performed to identify any radionuclidic impurities, particularly long-lived radionuclides. These results indicate a 89.3% and 94% reduction in impurities for ^{nat}Ni and ⁶¹Ni on niobium backing materials with respect to silver backing materials when utilizing the methods disclosed herein. ICP-MS measurements were performed on the product of cold dissolutions by Labor Veritas in Switzerland to monitor elemental impurities present in product according to ICH-Q3D. All detected impurities were within regulated ICH-Q3D concentrations (see ICH-Q3D Guidelines, page 25).

The plating of highly enriched ⁶¹Ni was also enabled with the same plating parameters as described above, for a higher yield and industrial production using proton irradiation (typically at 80 μA to 100 μA, 13 MeV protons for 1 hour to 2 hours and up to one half-life of ⁶¹Cu).

### 5.1.6. Purity and Activity Evaluations of [⁶¹Cu]CuCl₂ Compositions Prepared from ^{nat}Ni(d,n)⁶¹Cu and ⁶⁰Ni(d,n)⁶¹Cu Using Nb-Backed Coins

This example presents information on the activity of the produced ⁶¹Cu generated using the Nb backing, Ni electrodeposited coins of the present disclosure; alongside cobalt radioisotopes, that were produced with deuteron irradiation using the coin comprising a natural nickel target and the coin comprising enriched ⁶⁰Ni as target, i.e., ^{nat}Ni(d,n)⁶¹Cu and ⁶⁰Ni(d,n)⁶¹Cu, respectively. The irradiated materials were dissolved and purified as described above.

The obtained and purified ⁶¹Cu product and waste generated during purification from the products of deuteron irradiation of natural nickel/Nb coin and ⁶⁰Ni/Nb coin, respectively, was processed and analyzed by gamma-spectrometry and presented below.

TENDL-2019 is based on thick target yield calculations using isotopic abundancy of natural nickel/Nb coin and enriched ⁶⁰Ni/Nb coin, respectively.

### 5.1.7. Radiocobalt Content

Table 5 contains activities of cobalt radioisotopes in the different fractions post FASTlab purification as a mean of three measurements (n=3 irradiations) using ^{nat}Ni/Nb target coin. The activities were extrapolated to a 3 h and 50 μA beam at EoB (end of bombardment)+2 h. The activity of [⁶¹Cu]CuCl₂ in these irradiations was determined experimentally and confirmed to be ~80% of TENDL-2019 based estimates.

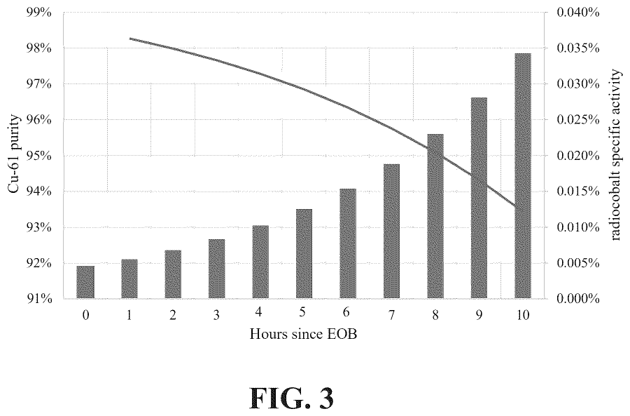

Activity of produced ⁶¹Cu for irradiation with deuteron at 8.4 MeV, 3 h at 50 μA at 80% efficiency (EoB+2 h): 3052 MBq. Also see FIG. 3 for the change in cobalt radioisotopes with time along with the corresponding change in ⁶¹Cu purity.

TABLE 5
Cobalt isotopes: natNi/Nb target coin

| | Cu | Ni | Co-waste | |
|---|---|---|---|---|
| | fraction | fraction | I + II | Half-life |
| Radionuclide | [Bq] | [Bq] | [Bq] | [days] |
| ⁵⁶Co | 118345 | 2696 | 2458071 | 77 |
| ⁵⁷Co | 0 | 0 | 474 | 272 |
| ⁵⁸Co | 95395 | 2145 | 1940192 | 71 |
| ⁶⁰Co | 124 | 3 | 2602 | 1925 |

Table 6 contains calculated activities of cobalt radioisotopes that would be obtained by using 99% enriched ⁶⁰Ni as target metal. The activities were extrapolated to a 3 h and 50 μA beam at EoB+2 h. The activity of ⁶¹Cu was calculated accordingly.

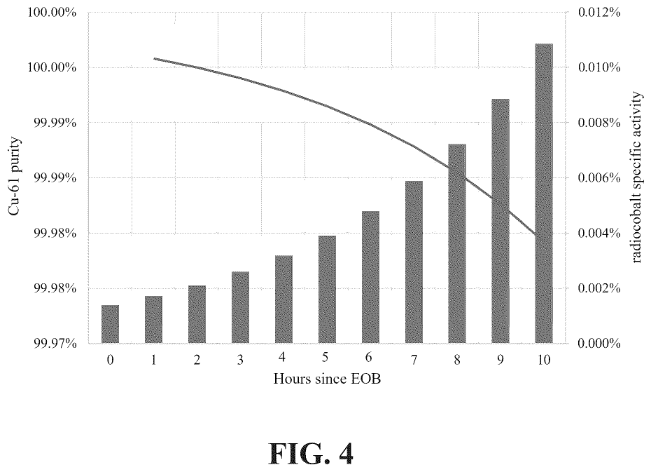

The activity of produced ⁶¹Cu with deuteron irradiation at 8.4 MeV, 3 h at 50 μA at 80% efficiency (EoB+2 h) was 11.552 MBq. Also see FIG. 4 for the change in cobalt radioisotopes with time and the corresponding change in ⁶¹Cu purity.

TABLE 6
Cobalt isotopes: enriched ⁶⁰Ni/Nb target coin.

| | ⁶¹Cu | | Separated | |
|---|---|---|---|---|
| | fraction | Separated | Co-waste | Half-life |
| Radionuclide | [Bq] | Ni [Bq] | I + II [Bq] | [days] |
| ⁵⁶Co | 365 | 8 | 7583 | 77 |
| ⁵⁷Co | 0 | 0 | 1793 | 272 |
| ⁵⁸Co | 242909 | 5463 | 4940424 | 71 |
| ⁶⁰Co | 0.5 | 0 | 11 | 1925 |

### 5.1.8. Activity and Chemical Purity

Based on measured activities (MBq) at different beam currents (μA) and timescales (5-60 minutes), the measured activity resulting from deuteron bombardment of ^{nat}Ni, ⁶⁰Ni and proton bombardment of ⁶¹Ni using the process described herein was found to be approximately >80% of the theoretical activity calculated using the TENDL-19 cross section database.

The activity of radiocobalt and other long-lived radionuclides was measured post-release (>3 weeks after bombardment). The EOB activity of the long-lived impurities was then extrapolated.

In Table 7, the extrapolated radiocobalt activity content and ⁶¹Cu purity of [⁶¹Cu]CuCl₂ solution produced by ^{nat}Ni as target metal for a 50 μA, 3 h deuteron irradiation after FASTlab purification were presented.

TABLE 7
Natural Ni/Nb Target Coin - Extrapolation of ⁶¹Cu activity
and purity in produced [⁶¹Cu]CuCl₂ solution.

| | Co | | | | % | |
|---|---|---|---|---|---|---|
| | species | | | | Purity | |
| | activity | | | | PET | |
| Hours | in Cu | ⁶¹Cu | ⁶⁴Cu | % | nuclides | % non-Cu |
| post | fraction | activity | activity | Purity | ⁶¹Cu + | radio- |
| EoB | [Bq] | [MBq] | [MBq] | ⁶¹Cu | ⁶⁴Cu | nuclides |
| 0 | 213864 | 4622 | 70 | | 99.995% | 0.00456% |
| 1 | 213784 | 3756 | 66 | 98.261% | 99.994% | 0.00559% |
| 2 | 213704 | 3052 | 63 | 97.979% | 99.993% | 0.00686% |
| 3 | 213624 | 2479 | 59 | 97.652% | 99.992% | 0.00841% |
| 4 | 213544 | 2015 | 56 | 97.274% | 99.990% | 0.01031% |
| 5 | 213465 | 1637 | 53 | 96.837% | 99.987% | 0.01263% |
| 6 | 213385 | 1330 | 50 | 96.332% | 99.985% | 0.01545% |
| 7 | 213305 | 1081 | 48 | 95.750% | 99.981% | 0.01890% |
| 8 | 213225 | 878 | 45 | 95.081% | 99.977% | 0.02309% |
| 9 | 213145 | 714 | 43 | 94.312% | 99.972% | 0.02817% |
| 10 | 213066 | 580 | 41 | 93.432% | 99.966% | 0.03434% |

Less than 0.03% non-Cu radioisotopes (⁵⁶Co and ⁵⁸Co) will be left in the copper fraction, assuming a product expiry time, e.g., >3 weeks post EoB. This value was lower than the limit allowed for Ga-68 cyclotron-produced as found in the Pharmacopeia (*0.1% at expiry for non-Ga radioisotopes):

The ⁶⁴Cu originating from ^{nat}Ni irradiation (content ~5% at expiry) will be the main impurity, reducing the radioisotopic purity of ⁶¹Cu product at longer irradiation times or shelf-life (illustrated as the grey curve in FIG. 3).

Table 8 and FIG. 4 show the extrapolated radiocobalt activity content and ⁶¹Cu purity of the produced [⁶¹Cu]CuCl₂ solution after FASTlab purification.

TABLE 8
⁶⁰Ni/Nb Target coin - Extrapolation of ⁶¹Cu activity
and purity in produced [⁶¹Cu]CuCl₂ solution.

| | Co | | | | % | |
|---|---|---|---|---|---|---|
| | species | | | | Purity | |
| | activity | | | | PET | |
| Hours | in Cu | ⁶¹Cu | ⁶⁴Cu | % | nuclides | % non-Cu |
| post | fraction | activity | activity | Purity | ⁶¹Cu + | radio- |
| EoB | [Bq] | [MBq] | [MBq] | ⁶¹Cu | ⁶⁴Cu | nuclides |
| 0 | 243275 | 17498 | 0.378 | | 99.999% | 0.00139% |
| 1 | 243176 | 14217 | 0.358 | 99.996% | 99.998% | 0.00171% |
| 2 | 242977 | 11552 | 0.339 | 99.995% | 99.998% | 0.00210% |
| 3 | 242680 | 9386 | 0.321 | 99.994% | 99.997% | 0.00259% |
| 4 | 242285 | 7627 | 0.304 | 99.993% | 99.997% | 0.00318% |
| 5 | 241792 | 6197 | 0.288 | 99.991% | 99.996% | 0.00390% |
| 6 | 241201 | 5035 | 0.272 | 99.990% | 99.995% | 0.00479% |
| 7 | 240514 | 4091 | 0.258 | 99.988% | 99.994% | 0.00588% |
| 8 | 239731 | 3324 | 0.244 | 99.985% | 99.993% | 0.00721% |
| 9 | 238853 | 2701 | 0.231 | 99.983% | 99.991% | 0.00884% |
| 10 | 237882 | 2195 | 0.219 | 99.979% | 99.989% | 0.01084% |

Less than 0.01% non-Cu radioisotopes (⁵⁶Co and ⁵⁸Co) were left in the Cu fraction, assuming a product expiry time of 8 h post EoB. This value was ten times lower than the allowed limit for ⁶⁸Ga cyclotron-produced as found in the Pharmacopeia (0.1% at expiry for non-Ga radioisotopes*).

Less than 0.02% ⁶⁴Cu was left in the copper fraction at an expiry time of 8 h post EoB, one hundred times lower than the specification required for ⁶⁸Ga (2% Ga radioisotopes were allowed for ⁶⁸Ga).

### 5.1.9. Purity of Produced [⁶¹Cu]CuCl₂ from Ni/Nb Target Coins: Comparison with Commercially Available Radionuclides

In Table 9, a comparison of the regulatory specifications on the purity of commercially available radionuclides are given along with the characteristics of the high purity [⁶¹Cu]CuCl₂ produced from deuteron irradiation of natNi/Nb and enriched ⁶⁰Ni/Nb target coin (50 μA, 3 h) and after FASTlab purification described herein.

TABLE 9
Comparison between commercially available radionuclides and [⁶¹Cu]
CuCl₂ solution produced from irradiation of ^{nat}Ni/Nb coins and enriched ⁶⁰Ni/Nb coins.

| | | % Max | | | | |
|---|---|---|---|---|---|---|
| | | radioisotopes | % Max other | | % | |
| | % Purity at | of same | radioisotopes | | Purity | % Max other |
| | EoB + 2 | element at | at EoB + 2 | Dominant | at | radioisotopes |
| Radionuclide | hours | EoB + 2 hours | hours | impurities | expiry | at expiry |
| ¹¹¹In | 99.93% | 0.075% | | ⁶⁵Zn, | 99.85% | 0.15% |
| | | | | ^{114m}In | | |
| ¹⁸F | | | | ⁵⁶Co | 99.90% | 0.10% |
| ¹⁸F | | | | ⁵⁶Co | 99.99% | 0.01% |
| ⁶⁸Ga | 98% | 2% | 0.10% | | | |
| cyclotron | | | | | | |
| ⁶⁸Ga | 99.90% | | 0.001% | ⁶⁸Ge | | |
| generator | | | | | | |
| ¹⁷⁷Lu | 99.90% | 0.05% | | | | |
| ⁶¹Cu from | 97.27% | 3.16% | 0.013% | ⁵⁶Co, ⁵⁸Co | 95.08% | 5% |
| ^{nat}Ni | (EoB + 4 h) | | | | (EoB + | |
| | | | | | 8 h) | |
| ⁶¹Cu from | 99.99% | 0.009% | 0.004% | ⁵⁶Co, ⁵⁸Co | 99.98% | 0.02% |
| ⁶⁰Ni | (EoB + 4 h) | | | | (EoB + | |
| | | | | | 8 h) | |

As the first notable comparison, cyclotron production of ⁶⁸Ga from proton irradiation also produces long lived radionuclides, (see, e.g., Applied Radiation and Isotopes, 65(10), 1101-1107, IAEA-TECDOC-1863 Gallium-68 Cyclotron Production)⋅notably ⁶⁵Zn (half-life=244 days) from the ⁶⁶Zn(p,pn)⁶⁵Zn decay. With a roughly 0.365% of ⁶⁶Zn in an enriched ⁶⁸Zn starting target metal, about 770 Bq of ⁶⁵Zn will be produced from a 50 μA, 3 h beam with an energy of 13 MeV in a thick target (TENDL-2019 based calculations). Using natural Zn with 27.7% abundancy in ⁶⁶Zn, 58 kBq of ⁶⁵Zn will be produced in one run of 50 μA for 3 h beam.

Similar with [⁶¹Cu]CuCl₂ production, cyclotron production of [⁶⁴Cu]CuCl₂ from proton irradiation also produces long-lived cobalt radionuclides, namely, ⁵⁵Co, ⁵⁷Co, ⁵⁸Co, and ⁶⁰Co. (See, e.g., Nuclear Medicine & Biology, Vol. 24, pp. 35-43, 1997; Applied Radiation and Isotopes 68 (2010) 5-13). By operating with a degraded beam of below 13 MeV, ⁶⁰Co (from ⁶⁴Ni(p,na)⁶⁰Co) was reduced to 1 Bq per run of 50 μA, 3 h. With beam energies below 13 MeV, ⁵⁵Co, formed from the ⁵⁸Ni(p,a)⁵⁵Co reaction, will remain the main impurity (half-life=17.53 hours). The 170 Bq of the long-lived ⁵⁷Co was formed in about 170 Bq in these conditions mostly from ⁶⁰Ni(p,a)⁵⁷Co.

Note: These estimates were computed from thick target yields using TENDL-2019 cross section data and isotopic abundancy of enriched ⁶⁴Ni as follows: 0.00376% ⁵⁸Ni, 0.00298% ⁶⁰Ni, 0.0058%⁶ 6Ni, 0.135% ⁶²Ni, 99.858% ⁶⁴Ni.

### 5.1.10. Enriched ⁶¹Ni as Target Metal on Nb Backed Coins

⁶¹Cu was produced through the proton bombardment of ⁶¹Ni electroplated Nb backed coin via cyclotron equipped with a solid target system irradiating a highly pure Niobium coin plated with highly pure ⁶¹Ni (purity 99.42%). The proton beam currents used were up to 100 μA, and beam energy of 13 MeV. An aluminum beam degrader was used.

The solid target irradiated material was dissolved in a total volume of 7 mL of 6M HCl with the addition of 30% H₂O₂ in a heated dissolution chamber. The ⁶¹Cu was purified from metal and radiometal impurities via a GE Healthcare FASTlab 2 module through a tributyl phosphate resin cartridge and a tertiary-amine-based weak ionic exchange resin containing long-chained alcohols. The product was finally eluted in an ISO class 5 environment in 3 mL 0.05 M HCl through a sterile filter Millex 4 mm Durapore PVDF 0.22 μm into a sterile evacuated vial. The vial was handled with care using the appropriate shielding and can be stored at room temperature until use using appropriate shielding for transport and handling. The properties of the [⁶¹Cu]CuCl₂ solution were determined and are displayed below in Table 10.

TABLE 10
[⁶¹Cu]CuCl₂ produced from ⁶¹Ni.

| Parameter | Test Method | Specification | |
|---|---|---|---|
| Appearance | Visual inspection | Clear, colorless solution, | |
| | | free from particulate matter | |
| Volume | Weight measurement | 3 ± 0.3 | mL |
| Activity | Dose calibrator | 0.20-2 | GBq/mL |
| concentration | | | |
| (EoS) | | | |
| pH value | pH paper strips | 1-1.6 | |
| Radiochemical | Radio-TLC | ≥99% (as [⁶¹Cu]CuCl₂) | |
| purity | | | |
| Radionuclidic | γ-Spectrometry (in | γ-photons with energy | |
| identity | lab at EoB + 90 | peaks at: 283 | |
| | minutes) | keV ± 20 keV | |
| | | 511 ± 20 keV (eventually | |
| | | sum peak at 1022 | |
| | | keV ± 20 keV) | |
| | | 656 keV ± 20 keV | |
| | Half-life via dose | 200 ± 20 | min |
| | calibrator | | |
| Radionuclidic | γ-Spectrometry (sent | ≥99.9% | |
| purity | out, evaluated >3 | | |
| | weeks, values | | |
| | extrapolated to in lab | | |
| | at EoB + 90 minutes) | | |
| Bacterial | LAL test (Endosafe) | ≤17.5 | EU/mL |
| endotoxin | | | |

| | | |
|---|---|---|
| content | | |
| Chemical | ICP-MS (sent out, | Sum of |
| purity | evaluated >3 weeks, | impurities ≤15 μg/GBq |
| | values relevant to | Cu ≤ 0.5 μg/GBq |
| | in lab at EoB + 90 | Al ≤ 2 μg/GBq |
| | minutes as these do | Co ≤ 1 μg/GBq |
| | not change with time) | Fe ≤ 3 μg/GBq |
| | | Pb ≤ 1 μg/GBq |
| | | Ni ≤ 2 μg/GBq |
| | | Zn ≤ 1 μg/GBq |

*post-release (≥3 weeks)
^{#}measured periodically

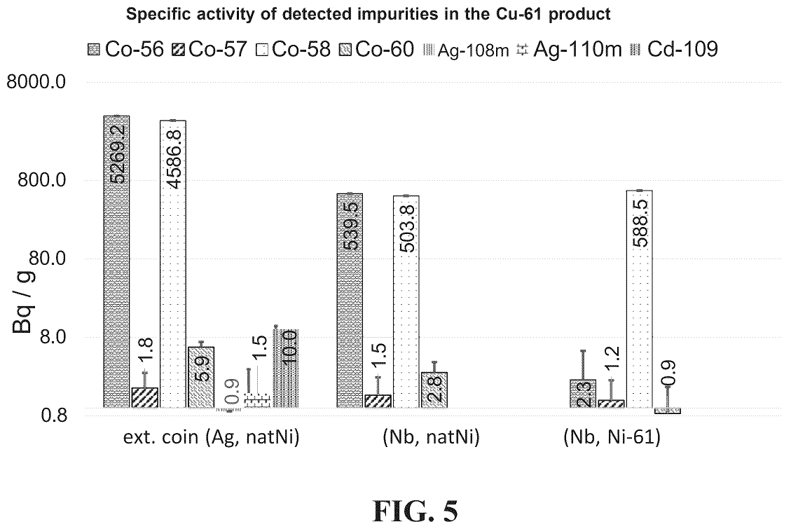

As shown in Table 11 and FIG. 5, commercially available [⁶¹Cu]CuCl₂ contains radionuclidic impurities, particularly high levels of ⁵⁶ Co and ⁵⁸Co, in addition to ^{110m}Ag and ¹⁰⁹Cd. Elimination of Ag and Cd isotopes from the Cu-61 product was achieved by replacing silver with niobium as backing material. There was a nine-fold reduction of ⁵⁶Co isotopes for natNi and ≥2000× reduction for Ni-61 (less shielding of radioactive waste is required). 5000 reduction of long-lived cobalt isotopes (earlier final disposal of the produced waste) was also observed. It was clear from the data below, that the radionuclidic purity of [⁶¹Cu]CuCl₂ produced by the methods described above to be superior to previously known methods and products. The high levels of long-lived Co, Ag, and Cd radionuclides pose a radiation burden for the patient and a radioactive waste issue for consumables that have come in contact with the [⁶¹Cu]CuCl₂ product during radiopharmaceutical manufacturing and radiolabeling.

TABLE 11
Detailed radionuclidic impurities present in commercially
available ⁶¹Cu compared to high-purity [⁶¹Cu]CuCl₂
of the present disclosure, expressed in Bq/g.

| Coin | |
|---|---|
| Ext. | |
| Coins | Present Coins |

| | | | nat-Ni | nat-Ni | ⁶¹Ni |
|---|---|---|---|---|---|
| | | | on Ag | on Nb | on Nb |
| Elements | T_{1/2} | | Bq/g | | |
| Co-56 | 77.23 | days | 5269.2 | 539.5 | 2.3 |
| Co-57 | 271.74 | days | 1.8 | 1.5 | 1.2 |
| Co-58 | 70.86 | days | 4586.8 | 503.8 | 588.5 |
| Co-60 | 5.27 | years | 5.9 | 2.8 | 0.9 |
| Ag-108m | 439 | years | 0.9 | N/D | N/D |
| Ag-110m | 249.86 | days | 1.5 | N/D | N/D |
| Cd-109 | 462.6 | days. | 10 | N/D | N/D |

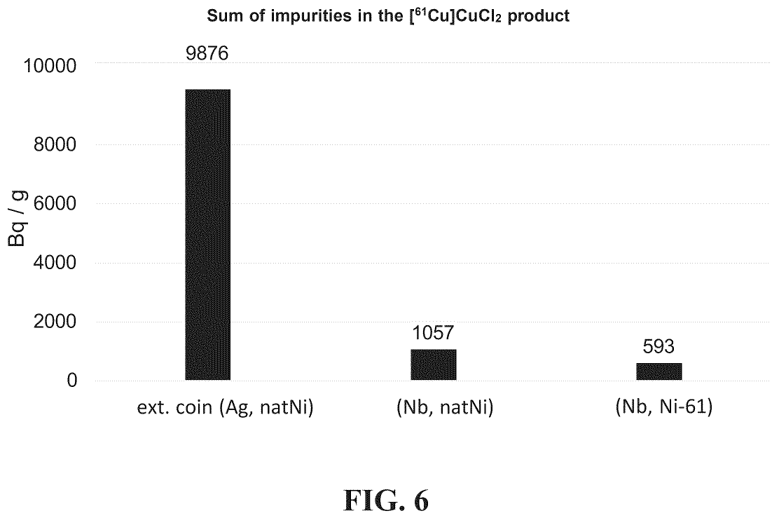

The total radionuclidic impurity profile was summed (Table 12 and FIG. 6). There was an 83% decrease in radionuclidic impurities. When present in the [⁶¹Cu]CuCl₂ product, these impurities can cause a radiation burden for the patient, waste issues, and degrade the quality of, e.g., a radiotracer or radiopharmaceutical. They can also interfere with the chelation process by competing with ⁶¹Cu, which affects the accurate radiolabeling of the tracer. An 89.3% reduction of impurities was observed upon changing the backing material from silver to the niobium backing provided herein and using the Ni plating methods described herein. An additional reduction of 46% was observed when using Ni-61 as starting material.

TABLE 12
Radionuclidic impurities in the produced [⁶¹Cu]CuCl₂.

| Coin | |
|---|---|
| Ext. Coins | Present Coins |

| | natNi | nat-Ni | ⁶¹Ni |
|---|---|---|---|
| | on Ag. | on Nb | on Nb |
| Bq/g | 9876 ± 1.5 | 1057 ± 1.8 | 593 ± 0.2 |

Consequent to the purity of the ⁶¹Cu at EoB and End of Synthesis (EoS, EoB+2), long-lived radionuclidic impurities decay slower and, thus, increase in concentration in relation to ⁶¹Cu at longer timescales. Thus, the impurity profile may vary greatly based on the isotopic enrichment of the raw material, purity, method, and process of producing a coin, which influences the type and amount of radionuclidic impurities in the finished [⁶¹Cu]CuCl₂ product.

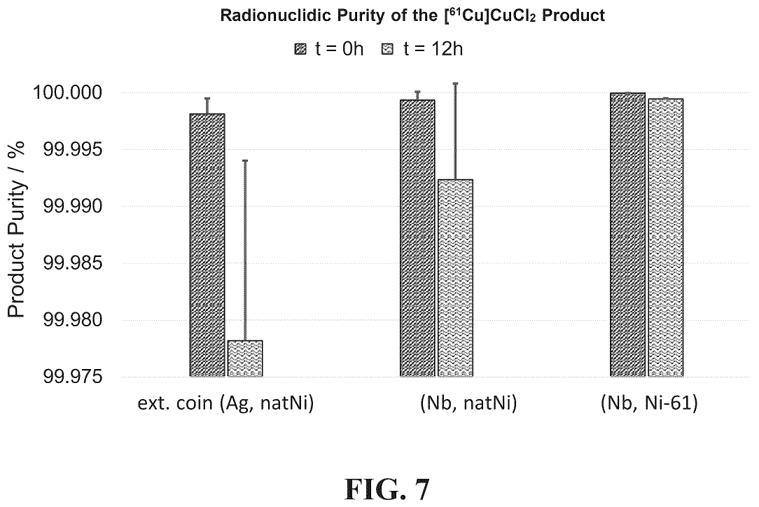

FIG. 7. contrasts the radionuclidic purity of [⁶¹Cu]CuCl₂ solution produced with commercially available natNi target metal on a Ag backing compared to the radionuclidic purity of [⁶¹Cu]CuCl₂ solution produced by irradiation of Ni target metal electroplated according to the present disclosure on high purity Nb backing when assessed by gamma spectrometry in Bq/g (summed radionuclidic impurities) at t=0 h and at t=12 h. The presented data highlight the superior quality of the [⁶¹Cu]CuCl₂ solution when produced by irradiation of Ni target coatings electroplated according to the present disclosure on high purity Nb backing, where the purity after 12 hours is still well above the purity limits set by pharmacopeia for similar radionuclides for medical use. “End of Production” or “EoP” refers to the end of the preparation of [⁶¹Cu]CuCl₂.

TABLE 13
Radionuclidic purity of commercially available ⁶¹Cu
compared to high-purity [⁶¹Cu]CuCl₂ of the present
disclosure as measured at EoP and EoP + 12 hours.

| Coin | |
|---|---|
| Ext. Coins | Present Coins |

| | nat-Ni | nat-Ni | ⁶¹Ni |
|---|---|---|---|
| | on Ag | on Nb | on Nb |
| Purity % t = 0 h | 99.998 | 99.999 | 99.9999 |
| Purity % t = 12 h | 99.978 | 99.993 | 99.999 |

### 5.1.11. Conclusion

The experimental activities of ⁶¹Cu produced after deuteron irradiation were about 80% of the theoretical yield as calculated from TENDL-2019 cross section data.

The main long-lived nuclides in the radioactive waste fraction from cyclotron production of ⁶¹Cu were radiocobalt species of ⁵⁶Co, ⁵⁷Co, ⁵⁸Co, and ⁶⁰Co. It was calculated that, after four years, ⁵⁶Co, ⁵⁷Co, and ⁵⁸Co will have decayed below regulatory clearance limits, LL*, leaving only ⁶⁰Co. *Clearance limits (LL) means the value corresponding to the activity concentration level of a material below which handling of this material is no longer subject to mandatory licensing or supervision.

The yield and purity of [⁶¹Cu]CuCl₂ prepared with niobium coins was improved by plating the niobium coins with 99% enriched ⁶⁰Ni or ⁶¹Ni. The purity of [⁶¹Cu]CuCl₂ product was higher as ⁶⁴Cu was be formed as a radioisotopic impurity. Additionally, the ⁵⁶Co and ⁶⁰Co contents were reduced by a factor of 100. ⁵⁷Co amounts increased (but were low activity) and ⁵⁸Co amounts doubled (but decay below LL before ⁵⁶Co/⁵⁸Co).

### 5.1.12. Batch Control of [⁶¹Cu]CuCl₂

Three representative batches of [⁶¹Cu]CuCl₂ solution were manufactured as described above (by irradiating a solid target consisting of highly pure ⁶¹Ni (purity ≥99.42%) plated on a niobium coin backing with a proton beam) and tested. The results of these analyses are presented in Table 14.

TABLE 14
Analyses of three batches of [⁶¹Cu]CuCl₂.

| | Specification | | Results | | | | | |
|---|---|---|---|---|---|---|---|---|
| Batch | — | | 1 | | 2 | | 3 | |
| Volume | 3 ± 0.3 | mL | 3.02 | mL | 3.00 | mL | 3.19 | mL |
| Activity | 0.50-2.00 | GBq/mL | 1.30 | GBq/mL | 1.29 | GBq/mL | 1.47 | GBq/mL |
| concentration | | | | | | | | |
| (EoP) | | | | | | | | |
| Apparent molar | 35-580 | MBq/nmol | 144.44 | | 144.44 | | 163.33 | |
| activity^{#} | | | MBq/nmol | | MBq/nmol | | MBq/nmol | |
| pH value | 1-1.6 | | 1.3 | | 1.2 | | 1.3 | |
| Radiochemical | ≥99% (as | | 100% | | 100% | | 100% | |
| purity | [⁶¹Cu]CuCl₂) | | | | | | | |
| Radionuclidic | γ-photons with | | 290 | keV | 286 | keV | 286 | keV |
| identity | energy peaks at: | | 506 | keV | 510 | keV | 510 | keV |
| | 283 keV ± 20 keV | | 654 | keV | 652 | keV | 656 | keV |
| | 511 ± 20 keV | | | | | | | |
| | (eventually sum | | | | | | | |
| | peak at 1022 | | | | | | | |
| | keV ± 20 keV) | | | | | | | |
| | 656 keV ± 20 keV | | | | | | | |
| Half-life | 200 ± 20 | min | 195.6 | min | 196.2 | min | 195.6 | min |
| Radionuclidic | ≥99.9% | | 99.99% | | 99.99% | | 99.99% | |
| purity* | | | | | | | | |
| Bacterial | ≤17.5 | EU/mL | ≤3.75 | EU/mL | ≤3.75 | EU/mL | ≤3.75 | EU/mL |

| | | | | |
|---|---|---|---|---|
| endotoxin | | | | |
| content | | | | |
| Chemical purity* | Sum of | Sum of | Sum of | Sum of |
| | impurities ≤20 | impurities | impurities | impurities |
| | μg/GBq | 2.40 μg/GBq | 2.11 μg/GBq | 2.72 μg/GBq |

*post-release.
^{#}measured periodically

### 5.1.13. Radionuclidic Purity

Radionuclidic purity is important in radiopharmacy since any radionuclidic impurities increase the radiation dose received by the patient and may also degrade the quality of any imaging procedure performed. For example, if significant levels of other radionuclides are present then biological distribution may be altered. Radionuclide samples contain some contaminants arising the production process or the decay of the primary radioisotope. Radionuclide impurities can occur as a result of the manufacturing process, for example, for nuclides produced by cyclotron there can be contaminants due to impurities in the target or by the energy of the reaction. In order to control the effects of these contaminants on the radiation dose received by the patient, limits are set on the maximum levels of contamination allowed. These limits are defined by governmental agencies, e.g., in pharmacopoeia monographs, and vary depending upon the radionuclide concerned and the physical decay characteristics of the likely contaminants. Measurement of radionuclidic purity may be performed high resolution using gamma-ray spectroscopy on samples well after bombardment. The activity of the long-lived isotopes is then extrapolated back to EoB, EoP, EoS, or even at expiration. High activity emitted from long lived radionuclidic impurities greatly increases the cost and complexity of managing the disposal of all consumables that come into contact with the nuclide composition.

Through the deuteron irradiation of natural nickel and ⁶⁰Ni, and proton irradiation of ⁶¹Ni, long-lived isotopes of cobalt are produced: ⁵⁶Co, ⁵⁷Co, ⁵⁸Co and ⁶⁰Co. Other long-lived radionuclides such as ^{110m}Ag, ^{108m}Ag and ¹⁰⁹Cd are produced through the irradiation of commonly used silver backing material, which are dissolved along with starting material during the purification process. Due to their long half-lives, the proportion of these radionuclides increases with time compared to the ⁶¹Cu, decreasing the radionuclidic purity of the product, especially at later time points when using ^{nat}Ni as a starting material. Though most cobalt isotopes can be separated in the purification process, the ^{110m}Ag, ^{108m}Ag and ¹⁰⁹Cd end up in the ⁶¹Cu fraction and nickel solution that is further used in recycling of irradiated target coating. The long-lived radionuclides become problematic when considering the radiation burden to the patient and the accumulation of radioactive waste. Third-party coin manufacturers did not publish the contamination from the non-niobium coin backings (e.g., silver). As provided by the present disclosure, the method of making and using coins comprising niobium represents an advantage, e.g., in view of the radionuclidic and chemical purity of samples produced following subatomic particle bombardment, isolation, and purification. A detailed comparison of the known ⁶¹Cu products (prepared via Ag backings and prior art methods of plating the target) to ⁶¹Cu as provided by the present disclosure is provided below.

With these factors in mind, a niobium backing material was chosen due to its inert nature to acids at room temperature and at elevated temperatures. This characteristic allows the niobium backing material to resist the acid medium used during the dissolution and purification process. By doing so, higher radionuclidic and chemical purity can be achieved in the radiometal aqueous solution, eventually resulting in higher purity for the radiopharmaceutical prepared from the desired ⁶¹Cu isotope. Although plating methods of niobium exist, the element has not yet been used for radionuclide production due to the poor adhesion of the plated Ni material (as discussed above). The Ni (or ⁶⁸Zn for the production of ⁶⁸Ga) requires sufficient adhesion for the coin to survive thermal loads (1200 W) during irradiation and pneumatic shuttle acceleration at 5 bar to 7 bar of pressure and abrupt stop at the head. On the other hand, however, the plated Ni (or Zn) must dissolve sufficiently during the dissolution and purification process. Attempts were made to plasma-coat niobium backings for plating nickel (Ni). However, this process resulted in losses and incomplete dissolution of Ni from the niobium backing. The thermal processes involved in plasma coating altered the grain structure of the niobium backing material, leading to a strong bond between the plated nickel and niobium. This strong bond made it difficult for the nickel to fully dissolve, causing losses. The plasma coating process itself resulted in very high losses in target coating, rendering the process not viable for use, especially with very expensive highly enriched target metals. The main reference to this summary is the IAEA documentation regarding cyclotron radionuclide production, IAEA RADIOISOTOPES AND RADIOPHARMACEUTICALS, REPORTS, No. 1. (INTERNATIONAL ATOMIC ENERGY AGENCY VIENNA, 2016) Additionally, a monetary evaluation regarding the procurement costs of niobium utilized as a backing material displays a 40% lower cost in comparison to commonly used backing materials such as gold, silver, and platinum where costs range from €80 to €120 per backing material (single coin).

Parallel to this, elements pertaining to the radiochemical purity of the labelling process are controlled by manufacturing the plating solution under controlled conditions described herein. By procuring the plating solution from a raw base material of, e.g., nickel, the possibility of contamination is now independent from outside sources and suppliers. Such material and equipment used in these cases are inert glass beakers and falcon tubes (ensured to not contain any undesirable substances), TraceSelect pure water, pure reagents (trace-metal grade), inert coin adapter and electrolytic cell (on the electroplating unit), etc. Through this, the contaminants of trace metals can be minimized reduced or avoided all together. This difference between 99.9% purity and 99.99% purity plays a role in the resulting chemical purity of a radionuclide and therefore in the radiochemical purity of, e.g., a radiotracer or radiopharmaceutical prepared from the radionuclide, where the presence of cold Cu, Zn, Fe, Sn, Ti, or Al or any salt thereof are an issue as they will compete for binding to the chelator in the tracer along with the desired radionuclide (⁶¹Cu).

Robustness of plating was tested through a drop and scratch test. This assessment ensures that the electrodeposited substrate on the backing will survive mechanical impacts of the shuttling system and established an increased probability of survivability under the cyclotron beam.

In certain embodiments, coins were irradiated with 8.4 MeV deuterons for an average duration of 120 mins at a range of 40 μA to 45 μA or with 13.2 MeV deuterons at 40 μA to 45 μA using an ARTMS or GE shuttling system on a GE PET Trace cyclotron.

In certain embodiments, the coins were irradiated with 8.4 MeV deuterons for an average duration of 120 mins at a range of 40 μA to 45 μA or with 10 μA to 100 μA 13 MeV protons using an ARTMS or GE shuttling system on a GE PET Trace cyclotron.

Dissolution of Ni from the niobium backing was accomplished via the utilization of a dissolution system in 10 M HCl. The subsequent ⁶¹Cu was then purified with two subsequent ion exchange resins in a FASTlab synthesis unit. The processing time for these purifications can reach up to 60 minutes.

The resulting [⁶¹Cu]CuCl₂ solution of the plated material has an average activity of 1.7-4.5 GBq. This activity was measured using a dose calibrator and its radionuclidic purity by a calibrated gamma spectrometer e.g., at PSI in Switzerland.

Gamma spectrometry measurements were performed to identify any radionuclidic impurities, particularly long-lived radionuclides. These results indicate an 89.3% and 94% reduction in impurities for ^{nat}Ni and ⁶¹Ni on niobium backing materials with respect to silver backing materials when utilizing the methods disclosed herein. ICP-MS measurements are performed on the product of cold dissolutions by Labor Veritas in Switzerland to monitor elemental impurities present in the product according to ICH-Q3D. All detected impurities are within regulated ICH-Q3D concentrations (see ICH-Q3D Guidelines, pg 25).

The plating of highly enriched ⁶¹Ni was also enabled with the same plating parameters as described above, for a higher yield and industrial production using proton irradiation (typically at A to 100 μA, 13 MeV protons for 20 minutes to 2 hours and up to one half-life of ⁶¹Cu).

Following automated transportation of the irradiated coin from the cyclotron to the hot cell docking station, the capsule was transferred to a QIS dissolution unit with tongs. The transmuted target metal was dissolved from the niobium backing material using 1:1 7M HCl: 30% H₂O₂ (ultratrace analysis, Merck) (4 mL). The acid-peroxide mixture is circulated, immersing the coin and target metal surface to dissolve all irradiated elements at 2 mL/min for about 23 minutes at about 60° C. When the target metal was fully dissolved, acidic solution containing the dissolved metal was withdrawn and the QIS system was flushed with 10M HCl (3 mL). The combined acidic solutions were then fed forward to the FASTlab purification unit.

For this reason, gamma spectrometry analyses were carried out on decayed samples (at least 10 half-lives of ⁶¹Cu, corresponding at the earliest to 1.4 d after EoP). The analysis was performed using a high-purity germanium (HPGe) detector GEM30-70 from Ortec.

### 5.1.14. Activity Concentration

The activity of the [⁶¹Cu]CuCl₂ solution was quantified using a dose calibrator following Ph. Eur. 2.2.66 guidelines. The test was carried out at the end of the radionuclide production (EoP) using a certified dose calibrator, selecting the ⁶¹Cu measuring channel. The weight of the [⁶¹Cu]CuCl₂ solution was measured using an analytical scale. Assuming a density of the aqueous solution of 1.0 g/mL, the weight is converted into a volume. The activity concentration, expressed in GBq/mL, is then calculated by dividing the activity by the volume.

### 5.1.15. Apparent Molar Activity

The suitability of the [⁶¹Cu]CuCl₂ solution for radiolabeling was ensured by determining the apparent molar activity. This test assessed the impact of competing trace metals on ⁶¹Cu chelation by quantifying the minimum amount of a given chelator required for efficient radiolabeling. The test can be performed with various chelators that can complex copper (e.g., NOTA, DOTA, NODAGA). Among them, NODAGA has been assessed for ⁶¹Cu molar activity. The test measures the percentage of complexation by radio-TLC after reacting a fixed amount of [⁶¹Cu]CuCl₂ solution with different amounts of chelator (titration). The results are then plotted (x axis (logarithmic): nmol of chelator; y axis: % of complexation), and a sigmoidal curve is obtained. The EC₅₀ point is extrapolated, corresponding to the amount of chelator needed to achieve the 50% of complexation. The apparent molar activity is calculated by dividing the ⁶¹Cu activity used extrapolated at the end of production (EoP) by 2 times the EC₅₀ value.

This test was not performed as a routine analysis, but when changes occur in 1) the grade of a chemical employed for the manufacturing of the [⁶¹Cu]CuCl₂ solution, 2) the quality of a consumable employed for the manufacturing of the [⁶¹Cu]CuCl₂ solution, 3) the target coin manufacturing process, and 4) if poor radiolabeling yields were observed. The AMA test is specific to a chelator and labelling conditions (e.g., buffer concentration and pH) and has thus to be repeated when these parameters are modified.

### 5.1.16. pH

The pH value was determined by colorimetric evaluation using pH paper strips with a narrow range (pH interval 0-2.5) exposed to a 10 μL of the [⁶¹Cu]CuCl₂ solution. The color of the paper strips was compared to the reference color scale displayed on the strips container.

### 5.1.17. Radiochemical Purity (Radio-TLC)

The radiochemical purity of a [⁶¹Cu]CuCl₂ solution prepared as described herein was determined by radio-TLC following Ph. Eur. 2.2.66 guidelines. The test determined the percentage of ⁶¹Cu present in the desired ionic form, namely as free Cu²⁺. The retention factor (R_{f}) was determined as the distance from the origin to the peak divided by the distance from the origin to the solvent front. Ionic [⁶¹Cu]Cu²⁺ migrated to the solvent front (R_{f}=0.8-1.0), while colloidal [⁶¹Cu]Cu(OH)₂ remained at the point of application (R_{f}=0.0-0.2). The specification required that ≥99% of the whole radioactivity detected was present in the ionic form [⁶¹Cu]Cu²⁺

For the test, 2 μL of the radioactive solution was applied at 1 cm away from the lower end of an iTLC paper plate (8×1 cm), consisting of glass microfiber chromatography paper impregnated with silica gel. After drying, the iTLC plate was placed in a glass chamber where it was run in a 0.1 M citrate buffer solution (pH 5). When the solvent front had reached a distance of approximately 1 cm from the top of the iTLC plate, the plate was removed from the chamber and scanned using a PET miniGita Star from Elysia-Raytest, equipped with a β-sensitive detector and controlled by the software Gina Star from Elysia-Raytest.

### 5.1.18. Gamma Spectrometry (Radionuclidic Identity)

The radionuclidic identity of a [⁶¹Cu]CuCl₂ solution prepared as described herein was confirmed by gamma spectrometry following Ph. Eur. 2.2.66 guidelines. The presence of the main γ-photons with energy peaks characteristic of ⁶¹Cu (listed in Table 15) was assessed. The table also lists the energy peak of the γ-photons belonging to ⁵⁸Co, which represents the main impurity detected in the test. The test was carried out at EoP using a Mucha Star multichannel analyser from Elysia-Raytest, equipped with a NaI detector, and controlled by the Gina Star from Elysia-Raytest software.

TABLE 15
Energy peaks characteristic of ⁶¹Cu and of its main impurity ⁵⁸Co.

| Peak | Energy (keV) |
|---|---|
| Cu-61 | 67 |
| Cu-61 | 283 |
| Beta peak | 511 |
| Cu-61 | 656 |
| Co-58 | 811 |
| Sum peak | 1022 |
| Cu-61 | 1185 |

### 5.1.19. Half-Life (Radionuclidic Identity)

The radionuclidic identity of a [⁶¹Cu]CuCl₂ solution prepared as described herein was further confirmed by half-life determination following Ph. Eur. 2.2.66 guidelines. The test was carried out at the end of the radionuclide production (EoP) using a certified dose calibrator from Comecer (model VDC-505), selecting the ⁶¹Cu measuring channel. The specification required the measured half-life value to be within a predefined range of the accepted half-life value (20%).

### 5.1.20. LAL Test (Bacterial Endotoxin Content)

The bacterial endotoxins were determined in a [⁶¹Cu]CuCl₂ solution prepared as described herein by limulus amoebocyte lysate (LAL) test following Ph. Eur. 2.6.14 guidelines. This system applies LAL kinetic chromogenic methodology that measures color intensity directly proportional to the endotoxin concentration in the sample. Each cartridge contained predefined amounts of LAL reagent, chromogenic substrate, and control standard endotoxin (CSE). The LAL reagent was mixed automatically by the device with the sample or the positive product control. The mixtures were incubated and then combined with the chromogenic substrate. For quantification, the optical density of the substrate was measured and analyzed against an internally archived standard curve. The analysis was performed in duplicate for the sample as well as for the positive product control. The system contained an internal printer to generate a report.

### 5.1.21. Bioburden

The bioburden of the aqueous [⁶¹Cu]CuCl₂ manufacturing process described herein was evaluated following Ph. Eur. 2.6.12 guidelines. This test enables the detection and quantification of the viable microorganisms present in the system prior to terminal sterilization and represents a good reference point for evaluating the degree of safety of the process. The manufacturing process was performed in completeness but used non-irradiated target coins for radiation protection. The collected non-radioactive solution was analyzed by the Membrane-Filtration Method. Half of the sample was passed through a membrane filter with a pore size of 0.45 μm. The filter was placed onto Soybean-Casein Digest Agar and incubated to determine the total aerobic microbial count (TAMC). The other half of the sample was passed through a membrane filter with a pore size of 0.45 μm. The filter was then placed onto Sabouraud Dextrose Agar and incubated to determine the total yeast and mold count (TYMC). The bioburden was expressed in colony-forming units (CFU).

### 5.1.22. Control of Starting Material

Reagents and starting materials used to manufacture the radionuclide ⁶¹Cu are provided in Table 16. The reagents employed in production were of TraceSelect grade to minimize the trace metal impurities. These impurities could impact radiolabeling, which may result in a poor complexation of ⁶¹Cu with the chelator.

TABLE 16
Materials used to purify and formulate [⁶¹Cu]CuCl₂.

| Material | Quality |
|---|---|
| ⁶¹Ni | 99.42% enrichment |
| Niobium coin backing | 99.9% purity |
| Hydrochloric acid | 30%, Ultrapure |
| Hydrogen peroxide | 30%, Suprapure |
| Sodium chloride | 99.99%, Suprapure |
| Water | Ultrapure |
| FASTlab cassette | Cassette's plastic components either |
| | sterile or tested for low bioburden |
| Solid Target Valve Box | Cassette's plastic components either |
| (STVB) cassette | sterile or tested for low bioburden |
| TBP and TK201 | Ion-exchange cartridge for purification |
| cartridges | |
| FASTlab water bag | Sterile WFI USP/Ph. Eur. |
| Vials | 20 mL sterile evacuated |
| Syringe needles | Sterile |
| Sterile filter Millex GV | 0.22 μm pore size, sterile |

At the end of the electroplating process, the target coin was examined using an optical microscope (20×, 50× and 250× magnification). All the steps of the coin manufacturing were performed using TraceSelect grade chemicals and metal-free consumables. The target coins were stored in a cool, dark environment in metal-free sealed containers.

### 5.1.23. Impurities

The impurities present in the irradiated starting material can undergo a nuclear reaction, leading to the formation of undesired radioactive species, as summarized in Table 17. Due to the high isotopic purity of the ⁶¹Ni (99.42% enrichment), these contaminations were limited.

TABLE 17
Composition of the ⁶¹Ni target coin
plating and main nuclear reactions.

| | |
|---|---|
| ⁶¹Ni composition | ⁵⁸Ni (0.004%), ⁶⁰Ni (0.3%), ⁶¹Ni (99.42%), |
| | ⁶²Ni (0.3%), ⁶⁴Ni (0.004%) |
| Desired nuclear reaction | ⁶¹Ni(p, n)⁶¹Cu |
| Contamination reactions | ⁶⁰Ni(p, α)⁵⁷Co (half-life: 271.74 d) |
| (selection of the main | ⁶¹Ni(p, α)⁵⁸Co (half-life: 70.86 d) |
| contributions) | ⁶¹Ni(p, n)⁶⁰Co (half-life: 1925 d) |

The presence of “cold” trace metal ions in the target coin material and reagents used during the dissolution and purification stages are to be limited. These trace metal ions can impact the subsequent radiolabeling processes by interfering with the complexation of ⁶¹Cu with the chelator. TraceSelect grade reagents and metal-free consumables are used with the aim of minimizing trace metal contamination.

The concentration of the trace metal ions in the [⁶¹Cu]CuCl₂ solution is determined by Inductively Coupled Plasma Mass Spectrometry (ICP-MS) (Table 18). ICP-AES can be used as alternative technique.

### 5.2. Example 2: Synthesis of FAP Inhibitors

### 5.2.1. Synthesis of (S)—N1-(2-aminoethyl)-N4-(4-((2-(2-cyano-4,4-difluoropyrrolidin-1-yl)-2-oxoethyl)carbamoyl)quinolin-6-yl)succinimide (1)

### Step 1: (S)-6-amino-N-(2-(2-cyano-4,4-difluoropyrrolidin-1-yl)-2-oxoethyl)quinoline-4-carboxamide (A)

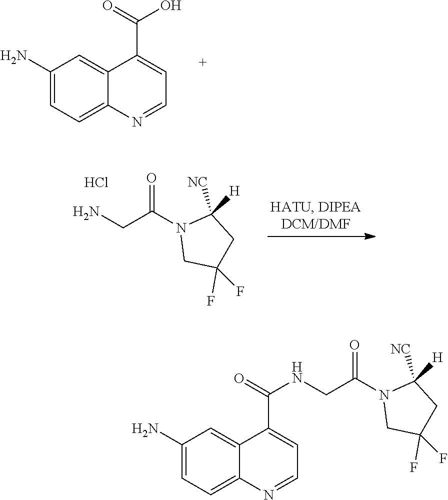 [SMILES: Nc1ccc2nccc(C(=O)O)c2c1 ; Cl ; N#C[C@@H]1CC(F)(F)CN1C(=O)CN ; N#C[C@@H]1CC(F)(F)CN1C(=O)CNC(=O)c1ccnc2ccc(N)cc12]

The two precursors (purchased from AstaTech) were dissolved together with HATU in DMF and then DCM was added. DIPEA was added dropwise and the reaction was monitored via LC/MS. The reaction was complete after less than 1 h. The crude product was concentrated, diluted with Water/ACN 85:15 and directly purified via HPLC (LCMS-2020 Shimadzu system equipped with a Gemini C-6 Phenyl column (10×250 mm, 5 μm particle size). The gradient used was 5-80% solvent B in 15 min (A=H₂O [0.1% TFA], B=ACN [0.1% TFA]) at a flow rate of 5.0 mL/min) to provide A as a pure red powder (38 mg, 84% yield).

### Step 2: Synthesis of (S)-4-((4-((2-(2-cyano-4,4-difluoropyrrolidin-1-yl)-2-oxoethyl)carbamoyl)quinolin-6-yl)amino)-4-oxobutanoic acid (B)

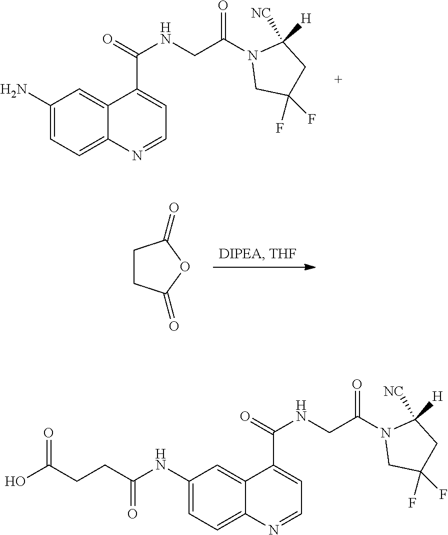 [SMILES: N#C[C@@H]1CC(F)(F)CN1C(=O)CNC(=O)c1ccnc2ccc(N)cc12 ; O=C1CCC(=O)O1 ; N#C[C@@H]1CC(F)(F)CN1C(=O)CNC(=O)c1ccnc2ccc(NC(=O)CCC(=O)O)cc12]

(S)-6-amino-N-(2-(2-cyano-4,4-difluoropyrrolidin-1-yl)-2-oxoethyl)quinoline-4-carboxamide (A) and succinic anhydride were dissolved in THF. DIPEA was added dropwise and the reaction was mixed overnight and checked via LC/MS. The crude product was directly purified via HPLC (LCMS-2020 Shimadzu system equipped with a Gemini C-6 Phenyl column (10×250 mm, 5 μm particle size). The gradient used was 5-80% solvent B in 8 min (A=H₂O [0.1% TFA], B=ACN [0.1% TFA]) at a flow rate of 5.0 mL/min) to afford B as a yellow powder (32.7 mg, 68% yield).

### Step 3: (S)—N1-(2-aminoethyl)-N4-(4-((2-(2-cyano-4,4-difluoropyrrolidin-1-yl)-2-oxoethyl)carbamoyl)quinolin-6-yl)succinimide (1)

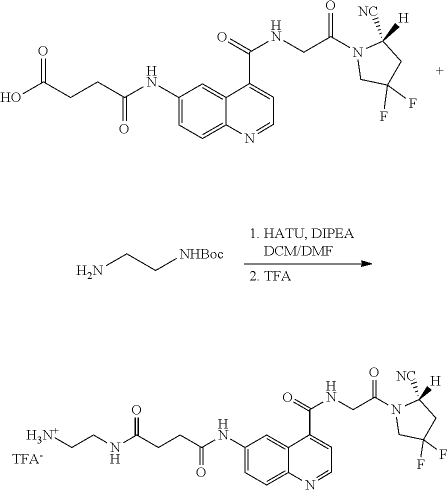 [SMILES: N#C[C@@H]1CC(F)(F)CN1C(=O)CNC(=O)c1ccnc2ccc(NC(=O)CCC(=O)O)cc12 ; CC(C)(C)OC(=O)NCCN ; N#C[C@@H]1CC(F)(F)CN1C(=O)CNC(=O)c1ccnc2ccc(NC(=O)CCC(=O)NCC[NH3+])cc12]

S)-4-((4-((2-(2-cyano-4,4-difluoropyrrolidin-1-yl)-2-oxoethyl)carbamoyl)quinolin-6-yl)amino)-4-oxobutanoic acid (B), HATU and the amine were dissolved in DCM and DMF. DIPEA was added dropwise and the reaction was mixed and checked via LC/MS. After completion, TIPS was added and TFA was added dropwise: first, the DIPEA was quenched. The deprotection step took over in 2 days. The crude material was used without further purification.

### 5.2.2. Synthesis of (S)—N1-(2-aminoethyl)-N4-(4-((2-(2-cyano-4,4-difluoropyrrolidin-1-yl)-2-oxoethyl)carbamoyl)quinolin-6-yl)-N4-methylsuccinamide (2)

Compound 2 was prepared as shown in Scheme 1:

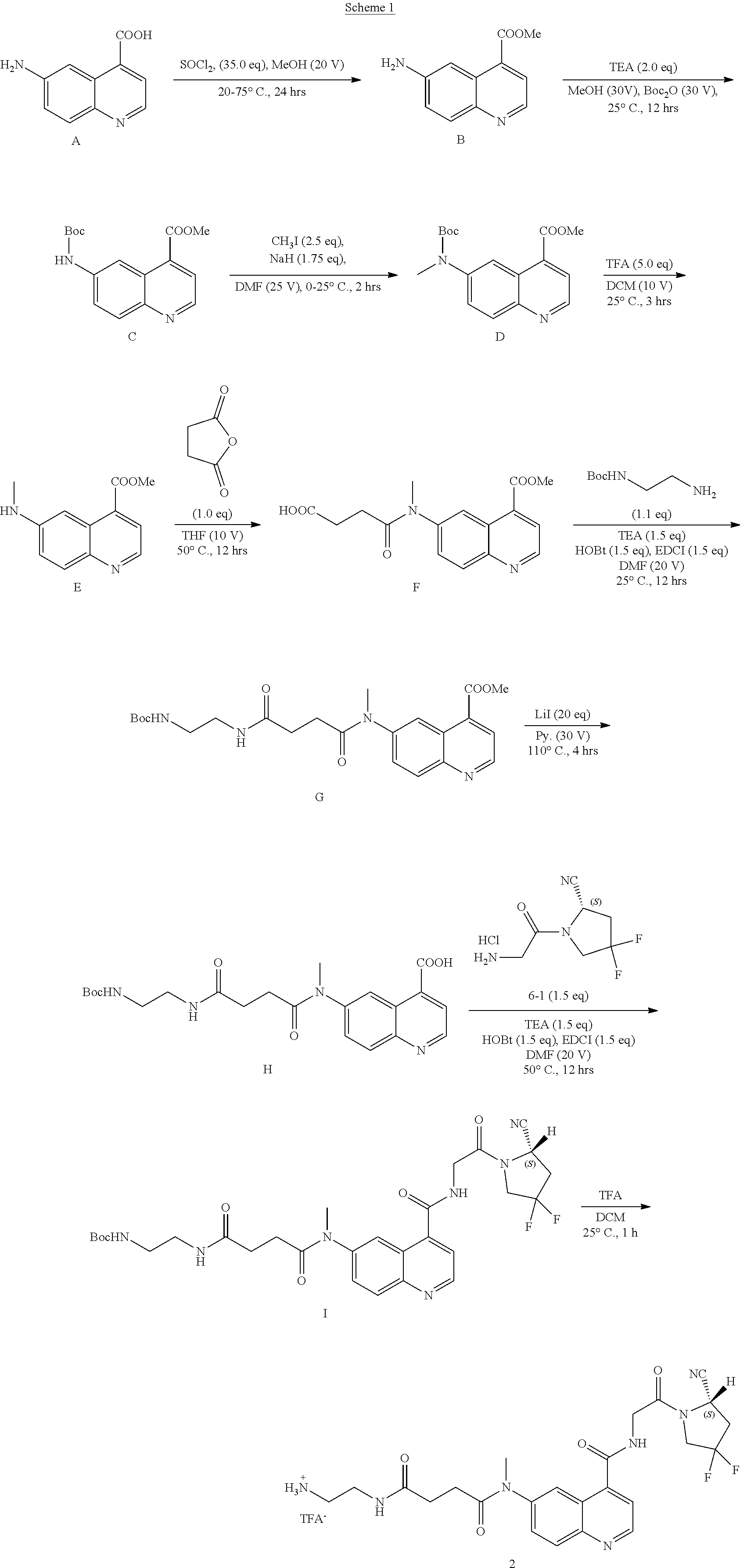

Step 1: To a mixture of compound A (4.17 g, 22.2 mmol) in MeOH (84.0 mL) was added SOCl₂ (26.4 g, 222 mmol, 16.1 mL) in one portion at 0-5° C. under N₂. The reaction was stirred at 0-5° C. for 0.5 h. The mixture was heated to 75° C. and stirred for 12 hrs. The mixture was added SOCl₂ (26.4 g, 222 mmol, 16.1 mL) and stirred for 12 hrs at 75° C. The mixture was added SOCl₂ (26.4 g, 222 mmol, 16.1 mL) and stirred for 12 hrs at 75° C. The mixture was added SOCl₂ (13.2 g, 111 mmol, 8.04 mL) and stirred for 12 hrs at 75° C. LC-MS showed one main peak with desired mass was detected. The mixture was concentrated in vacuum. The crude product was triturated with MeCN (300 mL) at 20° C. for 1 hr to afford compound B (7.05 g, crude) as a brown solid. ¹H NMR: (400 MHz, DMSO-d₆) δ 8.81 (d, J=4.8 Hz, 1H), 8.27 (d, J=8.8 Hz, 1H), 8.10 (d, J=4.8 Hz, 1H), 7.82 (s, 1H), 7.67 (d, J=8.0 Hz, 1H), 3.98 (s, 3H). LC-MS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, product: RT=1.262 min).

Step 2: To a solution of B (7.02 g, 34.7 mmol) in MeOH (100 mL), Boc₂O (100 mL) was added TEA (7.03 g, 69.4 mmol), the mixture was stirred at 25° C. for 12 hrs. LCMS showed compound B consumed and one peak of desired MS was detected. The mixture was concentrated in vacuum. The residue was purified by column chromatography (SiO₂, Petroleum ether/Ethyl acetate=100/1 to 1/1, compound C R_{f}=0.35) to obtain compound C (4.36 g, 41.5% yield) as a brown solid. ¹H NMR: (400 MHz, CDCl₃) δ 8.89 (d, J=4.4 Hz, 1H), 8.78 (d, J=2.4 Hz, 1H), 8.11 (d, J=9.2 Hz, 1H), 7.96-7.89 (m, 2H), 6.83 (s, 1H), 4.04 (s, 3H), 1.57 (s, 9H).

Step 3: To a solution of compound C (3.36 g, 11.1 mmol) in DMF (84.0 mL) was added NaH (778 mg, 19.5 mmol, 60% purity) in portions at 0° C., the mixture was stirred at 25° C. for 20 mins. Mel (3.94 g, 27.8 mmol) was added to the reaction mixture at 25° C. and stirred at 25° C. for 2 hrs. LCMS (ET60385-17-P1A3, Product RT=0.562 min) showed compound C consumed and one peak of desired MS was detected. The reaction mixture was cooled to 0° C. and quenched with brine (80.0 mL), extracted with EtOAc (3×100 mL). The organic layer was dried over sodium sulfate, filtered and concentrated in vacuum to obtain compound D (4.78 g, crude) as a brown solid.

Step 4: To a solution of compound D (4.78 g, 15.1 mmol) in DCM (50.0 mL) was added dropwise TFA (8.61 g, 75.5 mmol), the mixture was stirred at 25° C. for 12 hrs. LCMS showed compound D consumed and one peak of desired MS was detected. The reaction mixture was quenched with saturated NaHCO₃ (50.0 mL), extracted with DCM (3×40.0 mL). The organic layer was dried over sodium sulfate, filtered and concentrated in vacuum. The residue was purified by column chromatography (SiO₂, Petroleum ether/Ethyl acetate=100/1 to 1/1, product R_{f}=0.40) to obtain compound E (2.51 g, 76.8% yield) as a brown solid. ¹H NMR: ET60385-19-P1A1 (400 MHz, CDCl₃) δ 8.67 (d, J=4.4 Hz, 1H), 7.94 (d, J=9.2 Hz, 1H), 7.85 (d, J=4.4 Hz, 1H), 7.80 (d, J=2.4 Hz, 1H), 7.17-7.14 (m, 1H), 4.02 (s, 3H), 3.01 (s, 3H).

Step 5: To a solution of compound E (500 mg, 2.31 mmol) in THF (4.00 mL) was added tetrahydrofuran-2,5-dione (231 mg, 2.31 mmol), the reaction mixture was stirred at 50° C. for 12 hrs. LCMS showed compound E consumed and one peak of desired MS was detected. The mixture was concentrated in vacuum to obtain compound F (716 mg, crude) as a brown solid. ¹H NMR: ET60385-43-P1A1 (400 MHz, CDCl₃) δ 9.10 (d, J=4.0 Hz, 1H), 8.77 (d, J=2.4 Hz, 1H), 8.28 (d, J=8.8 Hz, 1H), 8.03 (d, J=4.0 Hz, 1H), 7.66-7.64 (m, 1H), 4.06 (s, 3H), 3.42 (s, 3H), 2.69-2.66 (m, 2H), 2.51-2.50 (m, 2H).

Step 6: To a solution of compound F (716 mg, 2.26 mmol) in DMF (7.00 mL) was added TEA (343 mg, 3.40 mmol), HOBt (458 mg, 3.40 mmol), EDCI (650 mg, 3.40 mmol) and tert-butyl N-(2-aminoethyl)carbamate (398 mg, 2.49 mmol), the reaction mixture was stirred at 25° C. for 12 hrs. LCMS showed compound F consumed and one peak of desired MS was detected. The reaction mixture was quenched with saturated NaHCO₃ (15.0 mL), extracted with DCM (25.0 mL×3) washed with brine (15.0 mL). The organic layer was dried over sodium sulfate, filtered and concentrated in vacuum to obtain compound G (1.33 g, crude) as a brown solid.

Step 7: To a solution of compound G (1.33 g, 2.90 mmol) in Py. (20.0 mL) was added LiI (7.86 g, 58.6 mmol), the mixture was stirred at 110° C. for 4 hrs. LCMS showed compound G consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 1%-30%, 20 min) to obtain compound H (647 mg, 50.1% yield) as an off-white solid.

Step 8: To a solution of compound H (617 mg, 1.39 mmol) in DMF (6.00 mL) was added DIEA (717 mg, 5.55 mmol), HATU (791 mg, 2.08 mmol) and compound 6-1 (587 mg, 2.08 mmol, 80% purity, HCl), the mixture was stirred at 25° C. for 1 hr. LCMS showed compound H consumed and one peak of desired MS was detected. The reaction mixture was quenched with saturated NaHCO₃ (15.0 mL), extracted with DCM (25.0 mL×3) washed with brine (15.0 mL). The organic layer was dried over sodium sulfate, filtered and concentrated in vacuum to obtain compound I (2.70 g, crude) as a brown solid.

Step 9: To a solution of compound I (2.70 g, 4.39 mmol) in DCM (10.0 mL) was added TFA (41.5 g, 364 mmol), the mixture was stirred at 25° C. for 1 hr. LCMS (ET60385-61-P1A4, Product RT=0.490 min) showed compound I consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 5%-35%, 20 min) to obtain compound 2 (260 mg, 11.1% yield, 97.3% purity) as a brown solid. LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=0.493 min).

### 5.2.3. Synthesis of (S)—N-(2-(2-cyano-4,4-difluoropyrrolidin-1-yl)-2-oxoethyl)-6-(4-oxo-4-(piperazin-1-yl)butanamido)quinoline-4-carboxamide (3)

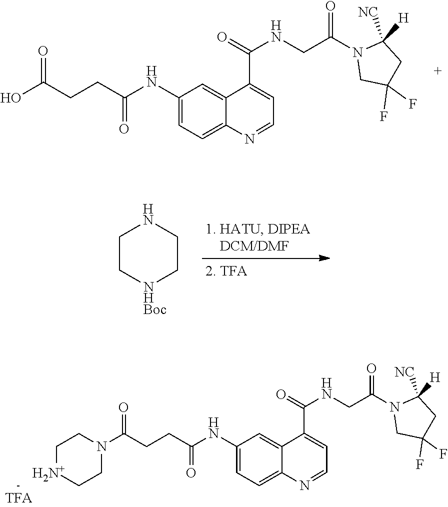

(S)-4-((4-((2-(2-cyano-4,4-difluoropyrrolidin-1-yl)-2-oxoethyl)carbamoyl)quinolin-6-yl)amino)-4-oxobutanoic acid, HATU and the amine were dissolved in DCM and DMF. DIPEA was added dropwise and the reaction was checked. When all the coupling occurred, the crude product was concentrated a bit and then TIPS was added. TFA was added dropwise and the mixture was checked via LC/MC until completion. Crude product (3) was used as such.

### 5.2.4. Synthesis of (S)—N-(2-(2-cyano-4,4-difluoropyrrolidin-1-yl)-2-oxoethyl)-6-(N-methyl-4-oxo-4-(piperazin-1-yl)butanamido)quinoline-4-carboxamide (4)

Compound 4 was prepared as shown in Scheme 2:

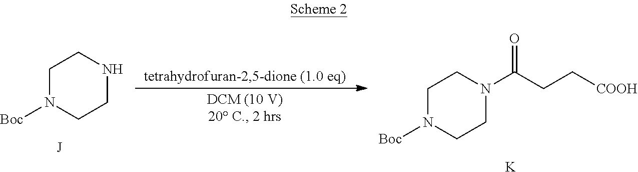

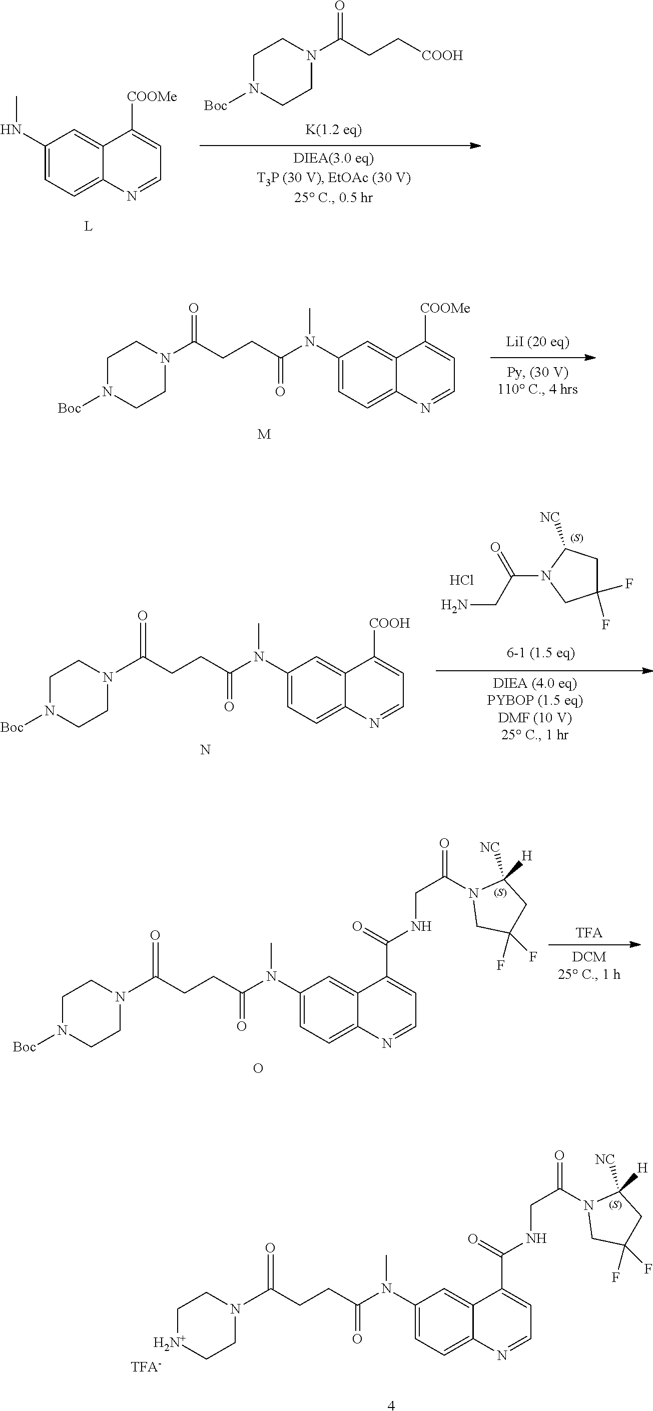

Step 1: To a mixture of compound J (10.0 g, 53.7 mmol) in DCM (70.0 mL) was added tetrahydrofuran-2, 5-dione (5.37 g, 53.7 mmol). The mixture was stirred for 2 hrs at 20° C. TLC (dichloromethane/methanol/AcOH=9/1/0.01, compound J R_{f}=0.0) showed the reaction was completed. The mixture was concentrated in vacuum. The residue was purified by silica gel chromatography (dichloromethane/methanol=100/1, 9/1) to afford compound K (4.75 g, 30.9% yield) as a white solid. ¹H NMR: (400 MHz, CDCl₃) δ 10.56-11.09 (m, 1H), 3.53-3.62 (m, 2H), 3.45 (s, 4H), 3.36-3.42 (m, 2H), 2.60-2.73 (m, 4H), 1.45 (s, 9H).

Step 2: To a solution of compound L (300 mg, 1.39 mmol) in EtOAc (10.0 mL) was added DIEA (537 mg, 4.16 mmol), compound K (476 mg, 1.66 mmol) and T₃P (11.2 g, 17.7 mmol, 50% purity), the reaction mixture was stirred at 25° C. for 0.5 hr. LCMS showed compound L consumed and one peak of desired MS was detected. Then reaction mixture is diluted with EtOAc (20.0 mL), washed with water (60.0 mL), saturated NaHCO₃ (60.0 mL), and brine (20.0 mL). The organic phase is dried over Na₂SO₄ and concentrated in vacuum to obtain compound M (716 mg, crude) as brown oil.

Step 3: To a solution of compound M (716 mg, 1.48 mmol) in Py. (20.0 mL) was added LiI (3.96 g, 29.5 mmol), the mixture was stirred at 110° C. for 4 hrs. LCMS showed compound M consumed and one peak of desired MS was detected. The mixture was concentrated in vacuum. The residue was purified by prep-HPLC (column: Welch Xtimate C18 250*100 mm #10 um; mobile phase: [water (NH₄HCO₃)-ACN]; B %: 1%-30%, 20 min) to obtain compound N (460 mg, 64.4% yield, 97.4% purity) as an off-white solid. LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=0.596 min)

Step 4: To a solution of compound N (460 mg, 977 umol) in DMF (5.00 mL) was added DIEA (505 mg, 3.91 mmol), PYBOP (763 mg, 1.47 mmol) and compound 6-1 (330 mg, 1.47 mmol, HCl), the mixture was stirred at 25° C. for 1 hr. LCMS showed one peak of desired MS was detected. The reaction mixture was quenched with saturated NaHCO₃ (15.0 mL), extracted with DCM (25.0 mL×3) washed with brine (15.0 mL). The organic layer was dried over Na₂SO₄, filtered and concentrated in vacuum to obtain compound O (2.10 g, crude) as brown oil.

Step 5: To a solution of compound O (2.10 g, 3.27 mmol) in DCM (10.0 mL) was added TFA (15.4 g, 135 mmol), the mixture was stirred at 25° C. for 1 hr. LCMS showed compound O consumed and one peak of desired MS was detected. The mixture was concentrated in vacuum. The residue was purified by prep-HPLC (column: Welch Xtimate C18 250*70 mm #10 um; mobile phase: [water (NH₄HCO₃)-ACN]; B %: 0%-40%, 20 min) to obtain compound 4 (196 mg, 11.0% yield) as an off-white solid.

### 5.2.5. Synthesis of FAPI-46

FAPI-46 was prepared as shown in Scheme 3:

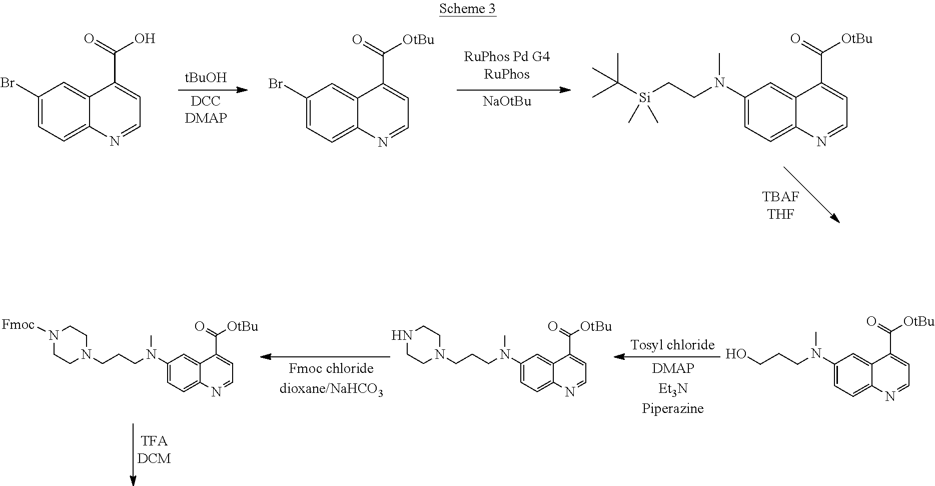

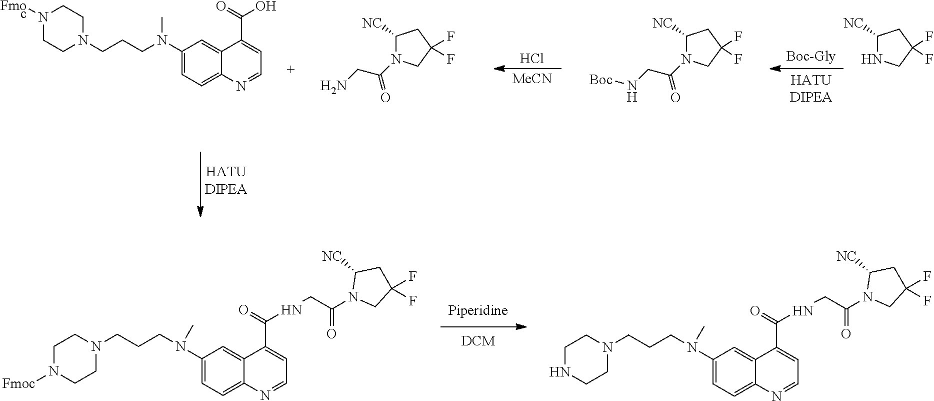

FAPI-46 can also be prepared according to the method described in WO 2019/154886A1.

### 5.3. Example 3: Synthesis of FAPI-NODAGA Conjugates

### 5.3.1. Synthesis of 2,2′-(7-((R)-1-carboxy-4-((2-(4-((4-((2-((S)-2-cyano-4,4-difluoropyrrolidin-1-yl)-2-oxoethyl)carbamoyl)quinolin-6-yl)amino)-4-oxobutanamido)ethyl)amino)-4-oxobutyl)-1,4,7-triazonane-1,4-diyl)diacetic acid ((R)-NODAGA-1)

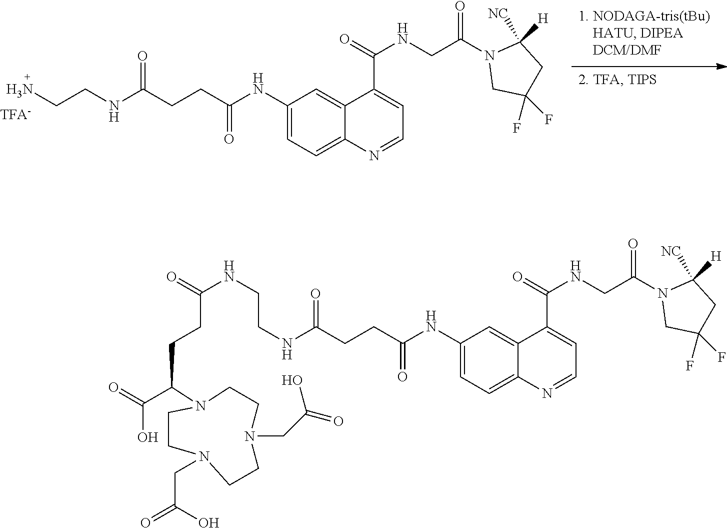 [SMILES: N#C[C@@H]1CC(F)(F)CN1C(=O)CNC(=O)c1ccnc2ccc(NC(=O)CCC(=O)NCCN)cc12 ; N#C[C@@H]1CC(F)(F)CN1C(=O)CNC(=O)c1ccnc2ccc(NC(=O)CCC(=O)NCCNC(=O)CC[C@H](C(=O)O)N3CCN(CC(=O)O)CCN(CC(=O)O)CC3)cc12]

To the (S)—N1-(2-aminoethyl)-N4-(4-((2-(2-cyano-4,4-difluoropyrrolidin-1-yl)-2-oxoethyl)carbamoyl)quinolin-6-yl)succinimide (1) crude solution, DIPEA was added dropwise to neutralize TFA. Then, HATU and NODAGA-Tris(tBu) were added dropwise as DMSO solution (150 μL). The reaction was complete after a few minutes. The crude product was concentrated and purified via HPLC. To the pure material, DCM, TIPS and TFA were added and the reaction was left for 1 day until completion and purified via HPLC to obtain 15.8 mg of (R)-NODAGA-1 as a pale yellow powder (Yield: 51%).

### 5.3.2. Synthesis of 2,2′-(7-((R)-1-carboxy-4-((2-(4-((4-((2-((S)-2-cyano-4,4-difluoropyrrolidin-1-yl)-2-oxoethyl)carbamoyl)quinolin-6-yl)(methyl)amino)-4-oxobutanamido)ethyl)amino)-4-oxobutyl)-1,4,7-triazonane-1,4-diyl)diacetic acid ((R)-NODAGA-2)

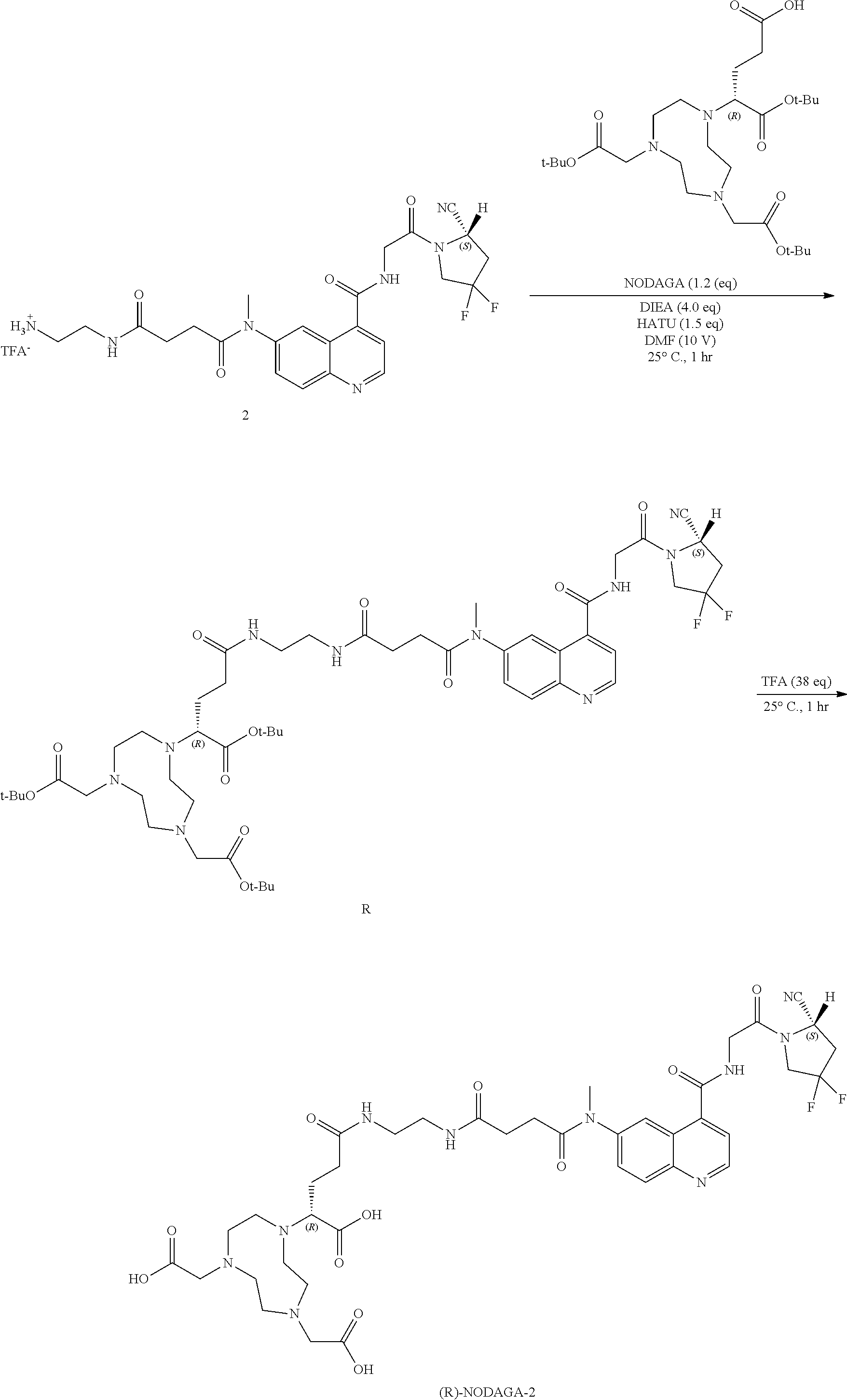

Step 1: To a solution of compound 2 (80.0 mg, 155 μmol) in DMF (1.00 mL) was added DIEA (80.2 mg, 620 μmol), HATU (121 mg, 232 μmol) and NODAGA-Tris(tBu) (101 mg, 186 μmol), the mixture was stirred at 25° C. for 1 hr. LCMS showed compound 2 consumed and one peak of desired MS was detected. The reaction mixture was quenched with saturated NaHCO₃ (4.00 mL), extracted with DCM (10.0 mL×3) washed with brine (10.0 mL). The organic layer was dried over sodium sulfate, filtered, and concentrated in vacuum to obtain R (310 mg, crude) was obtained as brown oil.

Step 2: To a solution of compound R (310 mg, 297 μmol) in TFA (1.29 g, 11.3 mmol) at 25° C., the mixture was stirred at 25° C. for 1 hr. LCMS showed compound R consumed and one peak of desired MS was detected. The mixture was concentrated in vacuum. The crude product on notebook page ET60385-73 (220 mg, crude) and ET60385-78 (206 mg, crude) was combined for further purification. The residue was purified by prep-HPLC (column: C18-1 150*30 mm*5 um; mobile phase:[water (TFA)-ACN]; B %: 5%-35%, 20 min) to obtained (R)-NODAGA-2 (10.01 mg, 3.30% yield, 96.9% purity, TFA) a brown solid. ¹H NMR: ET60385-73-P1A2 (400 MHz, D₂O) δ 9.14 (d, J=5.2 Hz, 1H), 8.32-9.30 (m, 2H), 8.02-7.98 (m, 2H), 5.18-5.14 (m, 1H), 4.38 (s, 2H), 4.33-4.24 (m, 1H), 4.20-4.10 (m, 1H), 3.76 (s, 4H), 3.51-3.31 (m, 4H), 3.25-3.12 (m, 12H), 3.03-2.87 (m, 6H), 2.49 (s, 3H), 2.30 (t, J=7.2 Hz, 2H), 2.03-1.85 (m, 1H). LCMS (ET60385-73-P1Z1, Product RT=1.610 min).

### 5.3.3. Synthesis of 2,2′-(7-((R)-1-carboxy-4-(4-(4-((4-((2-((S)-2-cyano-4,4-difluoropyrrolidin-1-yl)-2-oxoethyl)carbamoyl)quinolin-6-yl)amino)-4-oxobutanoyl)piperazin-1-yl)-4-oxobutyl)-1,4,7-triazonane-1,4-diyl)diacetic acid ((R)-NODAGA-3)

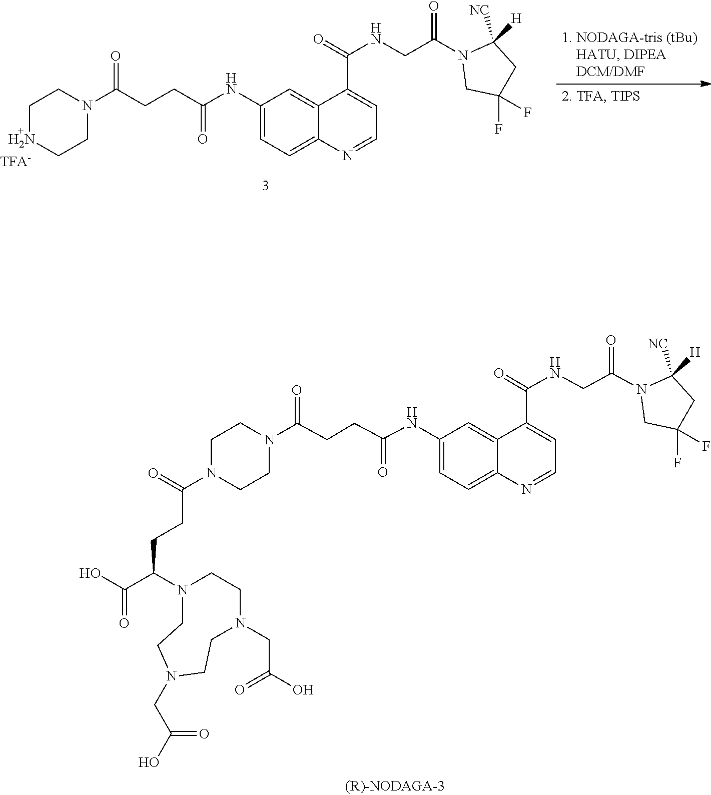 [SMILES: N#C[C@@H]1CC(F)(F)CN1C(=O)CNC(=O)c1ccnc2ccc(NC(=O)CCC(=O)N3CCNCC3)cc12 ; N#C[C@@H]1CC(F)(F)CN1C(=O)CNC(=O)c1ccnc2ccc(NC(=O)CCC(=O)N3CCN(C(=O)CC[C@H](C(=O)O)N4CCN(CC(=O)O)CCN(CC(=O)O)CC4)CC3)cc12]

To the (S)—N-(2-(2-cyano-4,4-difluoropyrrolidin-1-yl)-2-oxoethyl)-6-(4-oxo-4-(piperazin-1-yl)butanamido)quinoline-4-carboxamide crude solution, DIPEA was added dropwise to neutralize TFA. Then, HaTU and NODAGA-Tris(TBu) were added dropwise as DMSO solution (150 μL). The reaction was complete after a few minutes. The crude product was concentrated and purified via HPLC. To the pure material, DCM, TIPS and TFA were added and the reaction was left for 1 day until completion and purified via HPLC to obtain 15.8 mg of (R)-NODAGA-3 as a pale yellow powder (Yield: 26%).

### 5.3.4. Synthesis of 2,2′-(7-((R)-1-carboxy-4-(4-(4-((4-((2-((S)-2-cyano-4,4-difluoropyrrolidin-1-yl)-2-oxoethyl)carbamoyl)quinolin-6-yl)(methyl)amino)-4-oxobutanoyl)piperazin-1-yl)-4-oxobutyl)-1,4,7-triazonane-1,4-diyl)diacetic acid ((R)-NODAGA-4)

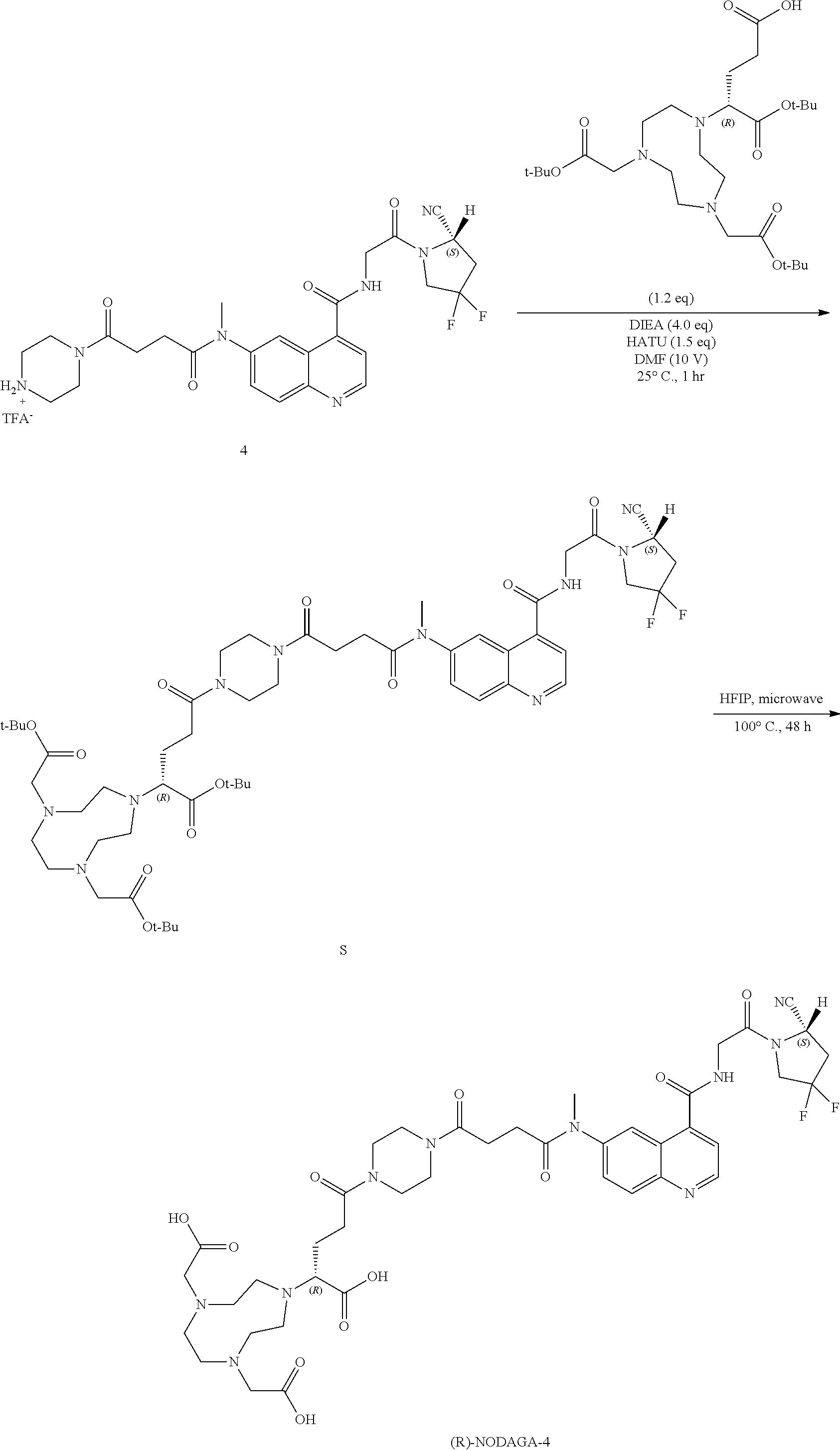

Step 1: To a solution of compound 4 (40.0 mg, 73.8 μmol) in DMF (0.50 mL) was added DIEA (9.55 mg, 73.8 μmol), HATU (57.6 mg, 110 μmol), and NODAGA-Tris(tBu) (48.1 mg, 88.6 μmol). The mixture was stirred at 25° C. for 1 hr. LCMS showed one peak of desired MS was detected. The mixture was concentrated in vacuum. The residue was purified by prep-HPLC (column: Waters Xbridge Prep OBD C18 150*40 mm*10 um; mobile phase: [water (NH₄HCO₃)-ACN]; B %: 50%-90%, 8 min) to obtain compound S (28.0 mg, 35.5% yield) as a white solid.

Step 2: Compound S (28.0 mg, 26.2 μmol) was taken up into a microwave tube in HFIP (4.41 mg, 26.2 μmol). The sealed tube was heated at 100° C. for 48 hrs under microwave. LCMS showed compound S consumed and one peak of desired MS was detected. The mixture was concentrated in vacuum. The residue was purified by prep-HPLC (column: Phenomenex Luna C18 75*30 mm*3 um; mobile phase: [water (TFA)-ACN]; B %: 5%-30%, 8 min) to obtain (R)-NODAGA-4 (9.01 mg, 36.9% yield, 96.6% purity, TFA) as an off-white solid. ¹H NMR: (400 MHz, D₂O) δ 9.10 (d, J=4.8 Hz, 1H), 8.31-8.27 (m, 2H), 8.00-7.97 (m, 2H), 5.15-5.12 (m, 1H), 4.35 (s, 2H), 4.26-4.22 (m, 1H), 4.17-4.15 (m, 1H), 3.75 (s, 4H), 3.60-3.50 (m, 9H), 3.22-3.09 (m, 18H), 2.67-2.58 (m, 6H), 2.07-1.96 (m, 2H). LCMS (LCMS-2020 Shimadzu system equipped with a Gemini C-6 Phenyl column (3.5×250 mm, 5 μm particle size). The gradient used was 5-80% solvent B in 8 min (A=H₂O [0.1% TFA], B=ACN [0.1% TFA]) at a flow rate of 1.0 mL/min, Product RT=1.640 min)

### 5.3.5. Synthesis of 2,2′-((R)-7-(1-carboxy-4-(4-(3-((4-((2-((S)-2-cyano-4,4-difluoropyrrolidin-1-yl)-2-oxoethyl)carbamoyl)quinolin-6-yl)(methyl)amino)propyl)piperazin-1-yl)-4-oxobutyl)-1,4,7-triazonane-1,4-diyl)diacetic acid (NODAGA-FAPI-46)

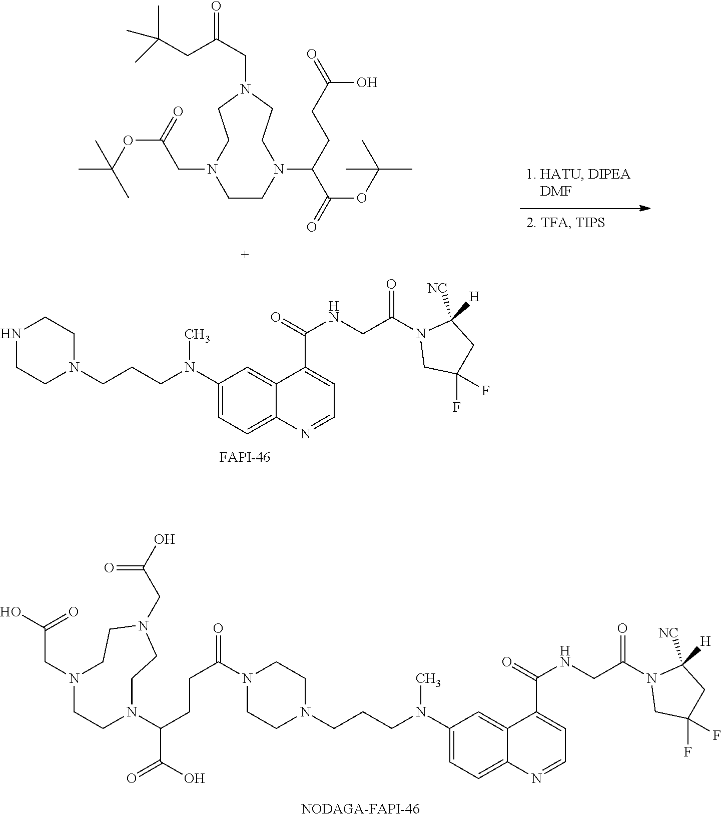 [SMILES: CC(C)(C)CC(=O)CN1CCN(CC(=O)OC(C)(C)C)CCN(C(CCC(=O)O)C(=O)OC(C)(C)C)CC1 ; CN(CCCN1CCNCC1)c1ccc2nccc(C(=O)NCC(=O)N3CC(F)(F)C[C@H]3C#N)c2c1 ; CN(CCCN1CCN(C(=O)CCC(C(=O)O)N2CCN(CC(=O)O)CCN(CC(=O)O)CC2)CC1)c1ccc2nccc(C(=O)NCC(=O)N3CC(F)(F)C[C@H]3C#N)c2c1]

(S)—N-(2-(2-cyano-4,4-difluoropyrrolidin-1-yl)-2-oxoethyl)-6-(4-oxo-4-(piperazin-1-yl)butanamido)quinoline-4-carboxamide, (R)-NODAGA(tris)tBu and HATU were dissolved in DCM+100 μL of DMF. DIPEA was added dropwise and the reaction was stirred for 2 h until completion (checked via LC/MS, method 15 to 80% in ACN). When no starting material was left and only a peak related to the product mass was observable (m/z=1025), TIPS and TFA (600 μL) were added. After 48 h, the reaction was complete. The crude was purified via HPLC (10-65% CAN in 15 min, rt=9.5) to afford 6.8 mg of a red powder (Yield: 36%).

### 5.4. Example 4: Cold Labeling

^{nat}Cu-NODAGA-1 and ^{nat}Cu-NODAGA-3

The ^{nat}Cu complexes were prepared by incubating each conjugate with 1.5-fold excess of ^{nat}CuCl₂×2 H₂O in ammonium acetate buffer, 0.5 M, pH 8 at 95° C. for 15 min. Uncomplexed ^{nat}Cu ions were eliminated by SepPak C-18 purification. The ^{nat}Cu-complexes were eluted with methanol, evaporated to dryness, re-dissolved in water and lyophilized. The purity of all complexes was confirmed by liquid chromatography and mass spectrometry (LC-MS). Table 18A presents the retention time (t_{R}), and the obtained mass (mass-to-charge ratio, m/z) of the ion [M+2H]²⁺ in comparison to the theoretical mass, confirming the identity of the formed ^{nat}Cu-complexed conjugates. The analysis was performed on a LC-MS (Shimadzu LC2020) system using Gemini C6 Phenyl 5 μm, 250×4.6 mm column and a gradient of 15-80% acetonitrile (0.1% TFA)/water (0.1% TFA) in 15 min, at a flow rate of 2 mL/min. LC-MS chromatogram data are provided in Table 18A.

TABLE 18A
Analytical for ^{nat}Cu conjugates.

| ^{nat}Cu-complexed conjugates | m/z calculated | m/z measured | t_{R} (min) |
|---|---|---|---|
| NODAGA-1 | 858.9 | 859 | 6.28 |
| ^{nat}Cu-NODAGA-1 | 920.2 | 920.3 | 9.77 |
| NODAGA-3 | 884.9 | 885 | 9.97 |
| ^{nat}Cu-NODAGA-3 | 946.9 | 946.5 | 9.94 |

m/z = mass-to-charge ratio of the ion [M + H]⁺;
t_{R} = retention time

^{nat}Cu-NODAGA-2 and ^{nat}Cu-NODAGA-4

The ^{nat}Cu complexes were prepared by incubating 1-1.5 mg of each conjugate with a 1.5-fold excess of CuCl₂ in 125-300 μL of ammonium acetate (0.5 M, pH 8). A pH check was performed in order to guarantee the necessary conditions for the reaction (pH≥5). The reaction mixture was incubated for 10 min at room temperature. Free metal ions were eliminated via HPLC (Shimadzu SCL-40, Phenomenex Jupiter Proteo C12 (90 μA, 250×4.6 mm) column using the gradient 15-80% B in 8 min (A=H₂O [0.1% TFA], B=ACN [0.1% TFA]) with a flow rate of 5 ml/min). The results are shown in Table 18B.

TABLE 18B
Analytical for ^{nat}Cu conjugates.

| Compound | m/z calculated | m/z measured | tR (min) |
|---|---|---|---|
| NODAGA-2 | 872.88 | 873 | 10.62 |
| ^{nat}Cu-NODAGA-2 | 934.41 | 934 | 9.44 |
| NODAGA-4 | 898.92 | 899 | 10.73 |
| ^{nat}Cu-NODAGA-4 | 960.45 | 960 | 10.46 |

m/z = mass-to-charge ratio of the ion [M + H]⁺;
tR = retention time

### 5.5. Example 5: Radiolabeling

[⁶¹Cu]Cu-NODAGA-1 and [⁶¹Cu]Cu-NODAGA-3

An aliquot of conjugate (3-6 nmol, 1 mg/mL in water) was diluted in 0.25-0.30 mL of ammonium (or sodium) acetate (0.5 M pH 8), followed by the addition of 0.1-0.7 mL [⁶¹Cu]CuCl₂ in 0.05 M HCl (70-240 MBq). The reaction mixture was incubated for 15 min at room temperature (approx. 20-25° C.). The pH of the reaction was between 5 and 6. Quality control was performed on a reverse-phase high performance liquid chromatography (RP-HPLC) connected to a radiodetector (radio-HPLC). Phenomenex Jupiter Proteo C12 (90 μA, 250×4.6 mm) column using the gradient 15-80% B in 8 min (A=H₂O [0.1% TFA], B=ACN [0.1% TFA]) with a flow rate of 1 mL/min. The results of the radio-HPLC are provided in Table 19 below.

[⁶¹Cu]Cu-NODAGA-2 and [⁶¹Cu]Cu-NODAGA-4

⁶¹Cu-labeled conjugates were prepared by incubating 1.5-3 nmol of the corresponding conjugate (as a 1 mg/mL solution) in 125-300 μL of ammonium acetate (0.5 M, pH 8) with 50-200 μL of [⁶¹Cu]CuCl₂ in 0.05 M HCl (33-70 MBq). A pH check was performed in order to guarantee the necessary conditions for the reaction (pH≥5). The reaction mixture was incubated for 10 min at room temperature. Quality control and stability studies were performed by Radio-HPLC on a Shimadzu SCL-40 connected to a GABI radioactivity-HPLC-flow-monitor 7-spectrometer (Elysia-raytest, Straubenhardt, Germany). Radioligands were analyzed using Phenomenex Jupiter Proteo C12 (90 μA, 250×4.6 mm) column using the gradient 15-80% B in 8 min (A=H₂O [0.1% TFA], B=ACN [0.1% TFA]) with a flow rate of 1 mL/min. The results are shown in Table 19.

TABLE 19
Radiochemical purity and retention time
(tR) of the ⁶¹Cu-labeled conjugates.

| Radiotracer | Radiochemical purity | t_{R} (min) |
|---|---|---|
| [⁶¹Cu]Cu-NODAGA-1 | ≥98% | 5.9 ± 0.2 |
| [⁶¹Cu]Cu-NODAGA-2 | ≥98% | 6.1 ± 0.2 |
| [⁶¹Cu]Cu-NODAGA-3 | ≥97% | 5.7 ± 0.2 |
| [⁶¹Cu]Cu-NODAGA-4 | ≥98% | 6.4 ± 0.2 |
| [⁶¹Cu]Cu -NODAGA-FAPI-46 | ≥95% | 5.7 ± 0.2 |

All conjugates were labeled with ⁶¹Cu in very high yield and purity. No further purification step was necessary to remove uncomplexed ⁶¹Cu from the reaction mixture, allowing direct use of the formed radiotracer.

### 5.6. Example 6: Partition Coefficient (Log D)

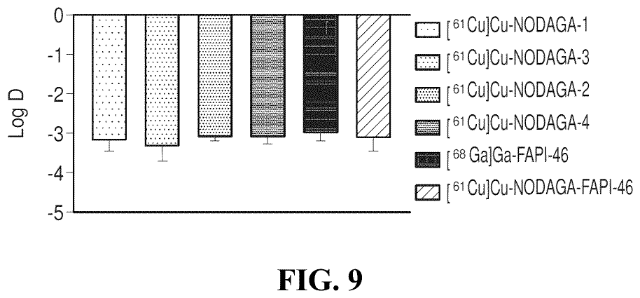

The lipophilic/hydrophilic character of the radiotracers was assessed by the determination of the distribution coefficient (D), expressed as log D (pH=7.4), between an aqua and an organic phase following the “shake-flask” method. In a pre-lubricated Eppendorf tube, a pre-saturated mixture of 500 μL of 1-octanol and 500 μL of PBS pH 7.4 (phosphate-buffered saline) were added. An aliquot of 10 pmol in 10 μL of the radioligand was added to this mixture, shaken for 30 min, and then centrifuged at 3000 rcf for 10 min to achieve phase separation. Aliquots of 100 μL were removed from the 1-octanol and from the PBS phases, and the activity was measured in a γ-counter. The partition coefficient was calculated as the average log ratio value of the radioactivity in the organic fraction and PBS fraction. The results are presented in Table 20 and in FIG. 9.

TABLE 20
Lipophilicity expressed as the log distribution coefficient
D (log D_{O/PBS pH 7.4}) of ⁶¹Cu-labeled conjugates versus
⁶⁸Ga-labeled conjugates (reference radiotracers).

| Radiotracer | log D_{(O/PBS pH 7.4)} |
|---|---|
| [⁶¹Cu]Cu-NODAGA-1 | −3.17 ± 0.28 |
| [⁶¹Cu]Cu-NODAGA-3 | −3.32 ± 0.39 |
| [⁶¹Cu]Cu-NODAGA-2 | −3.09 ± 0.08 |
| [⁶¹Cu]Cu-NODAGA-4 | −3.12 ± 0.16 |
| [⁶¹Cu]Cu-NODAGA-FAPI-46 | −3.10 ± 0.34 |
| [⁶⁸Ga]Ga-FAPI-46 | −3.01 ± 0.18 |

Results are means ± standard deviation from a minimum of two separate experiments, each in triplicates.

### 5.7. Example 7: In Vitro hFAP Inhibition Assay

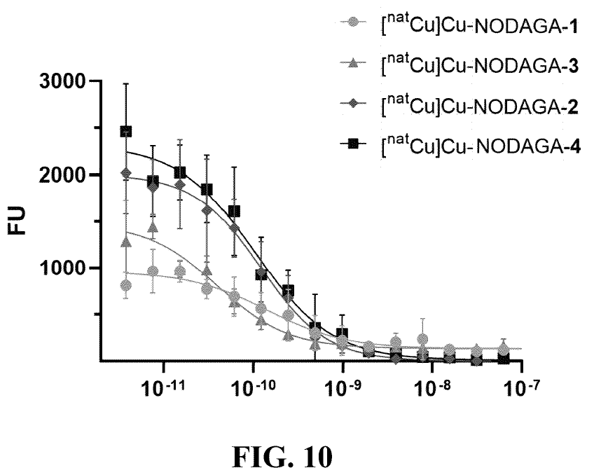

The enzymatic activity of hFAP on the substrate Z-Gly-Pro-AMC was measured at room temperature on a microtiter plate reader, monitoring the fluorescence at an excitation wavelength of 360 nm and an emission wavelength of 465 nm. The assay was performed by mixing the substrate (20 μM), hFAP (200 pM, constant), and the inhibitors in assay buffer (50 mM Tris, 1 M NaCl, 1 mg/mL BSA, pH=7.5), with serial dilution of the inhibitors ranging from 250 nM to 2 fM, 1:2 in a total volume of 20 μL. FAPI-46 was used as positive control. Experiments were performed in triplicate, and the mean fluorescence values were fitted using Graph Pad Pri-sm 9 (equation used: Y=Bottom+(Top−Bottom)/(1+((X\hat{ }HillSlope)/(IC50\hat{ }HillSlope)))). The IC₅₀ value is defined as the concentration of inhibitor required to reduce the enzyme activity by 50% after the addition of the substrate. The results are presented in Table 21 and FIG. 10.

TABLE 21
In Vitro Inhibition Assay

| Compound | IC₅₀ (pM) | 95% CI (pM) |
|---|---|---|
| [^{nat}Cu]Cu- NODAGA-1 | 141.3 | 71.9 to 230.0 |
| [^{nat}Cu]Cu-NODAGA-3 | 40.1 | 26.2 to 54.4 |
| [^{nat}Cu]Cu-NODAGA-2 | 120.5 | 88.0 to 157.8 |
| [^{nat}Cu]Cu-NODAGA-4 | 105.1 | 63.3 to 149.4 |

### 5.8. Example 8: In Vitro Cellular Uptake

⁶¹Cu-NODAGA-1 and ⁶¹Cu-NODAGA-3

The cellular uptake was studied in vitro using intact cells seeded in 6-well plates overnight. On the day of the experiment, the cells were washed and incubated with each ⁶¹Cu-labeled conjugate at different time points, either alone or in the presence of a blocking agent to distinguish between specific and non-specific uptake. At each investigated time point, the medium containing the unbound (free) radiotracer was removed, followed by two washing steps with ice-cold phosphate-buffered saline. The cells were then treated 2×5 min with ice-cold glycine solution (0.05 M, pH 2.8) to detach the cell surface-bound radiotracer (acid released). Afterwards, the cells containing the internalized radiotracer were detached with 1 M NaOH at 37° C. and collected for measurement. The amount of specific cell surface-bound and internalized radiotracer is expressed as percentage of the total applied activity, after subtracting the non-specific values. [⁶¹Cu]Cu-NODAGA-1, [⁶¹Cu]Cu-NODAGA-3 and [⁶¹Cu]Cu-NODAGA-FAPI-46 (0.2 nM) were assessed in HT-1080.hFAP (FAP-positive) and HT-1080.wt (FAP-negative) cells. Internalization and cell surface-bound fractions for the tested radiotracers are reported in Table 22. The values are expressed as % of the applied activity and refer to the specific uptake calculated after subtracting the non-specific values (measured in the presence of the non-FAP expressing cell line HT-1080.wt) from the total values (specific=total−non-specific).

TABLE 22
Cellular uptake and distribution

| Time | | | | | [⁶¹Cu]Cu- |
|---|---|---|---|---|---|
| Point | [⁶¹Cu]Cu- | [⁶¹Cu]Cu- | [⁶¹Cu]Cu- | [⁶¹Cu]Cu- | NODAGA- |
| [min] | NODAGA-1 | NODAGA-2 | NODAGA-3 | NODAGA-4 | FAPI-46 |
| | | Cell surface fraction | | | |
| 15 | 1.2 ± 0.3 | 1.2 ± 0.6 | 0.9 ± 0.3 | 1.0 ± 0.7 | 0.9 ± 0.3 |
| 60 | 1.4 ± 0.3 | 1.4 ± 0.5 | 1.2 ± 0.4 | 1.4 ± 0.4 | 1.2 ± 0.4 |
| 240 | 1.3 ± 0.2 | 1.3 ± 0.5 | 1.4 ± 0.4 | 0.9 ± 0.6 | 1.7 ± 0.4 |
| | | Internalized fraction | | | |
| 30 | 26.2 ± 3.5 | 20.7 ± 5.5 | 26.6 ± 4.9 | 22.2 ± 7.4 | 24.3 ± 2.3 |
| 60 | 29.9 ± 1.8 | 27.0 ± 6.9 | 36.9 ± 5.3 | 22.2 ± 5.1 | 36.1 ± 1.6 |
| 240 | 29.3 ± 1.8 | 28.3 ± 7.5 | 39.4 ± 4.8 | 17.4 ± 4.4 | 50.0 ± 6.0 |

[⁶¹Cu]Cu-NODAGA-2 and [⁶¹Cu]Cu-NODAGA-4

Upon thawing, HT-1080.hFAP (FAP-positive), HT-1080.wt (FAP-negative), HEK-293.hFAP and HEK-293.wt cells were kept in culture in MEM medium supplemented with fetal bovine serum (10%, FBS) and Penicillin-Streptomycin (1%) at 37° C. and 5% CO₂. For passaging, cells were detached using Trypsin-EDTA 0.05% when reaching 90% confluency and re-seeded at a dilution of 1:4/1:12 (HT-1080) or 1:10/1:20 (HEK-293).

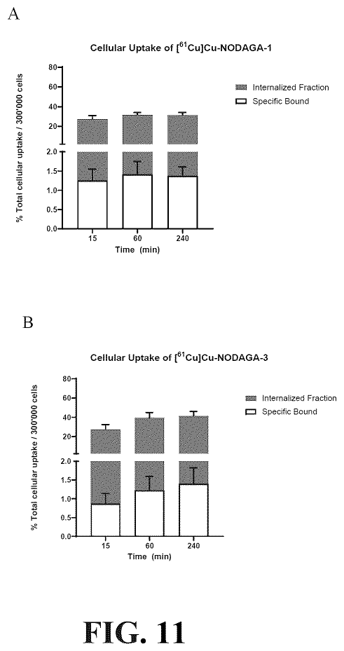

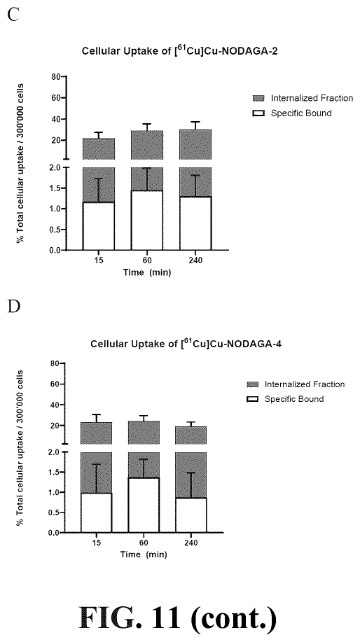

HT-1080.hFAP and HT-1080.wt cells were seeded in a 24-well plate at a concentration of 1.8×10⁵ cells/well in 400 μL of medium 24 hours before the experiment. The cells were then preconditioned in 360 μL of assay medium (MEM medium without supplements) at 37° C. for 60 min. 40 μL of a 2 nM solution of ⁶¹Cu-labeled radioligand was added and the cells were incubated at 37° C. The cellular uptake was interrupted at different time points (15 min, 1 hour and 4 hours), by washing twice with ice-cold PBS. Cell surface-bound radioligand was obtained by washing cells twice with ice-cold glycine buffer (pH 2.8), followed by a collection of the internalized fraction with 1 M NaOH. The activity in each fraction was measured in a γ-counter (Cobra II). The results are expressed as a percentage of the applied radioactivity, after subtracting the non-specific uptake in the HT-1080.wt cells (FIG. 11 and FIG. 12).

The ⁶¹Cu-labeled FAP radiotracers were fast and almost entirely internalized on cell expressing the human FAP at 37° C., with only a negligible amount remaining on the cell surface (cell membrane).

### 5.9. Example 9: Saturation Binding Experiment

Cell Membrane Preparation: HEK-293.hFAP cells were grown to confluence, mechanically disaggregated, washed with PBS (pH 7.4) and re-suspended in 20 mM of homogenization Tris buffer (pH 7.5) containing 1.3 mM EDTA, 0.25 M sucrose, 0.7 mM bacitracin, 5 μM soybean trypsin inhibitor, and 0.7 mM PMSF. The cells were homogenized using Ultra-Turrax, and the homogenized suspension was centrifuged at 500×g for 10 min at 4° C. The supernatant was collected in centrifuge tubes (Beckman Coulter Inc., Brea, CA, USA). This procedure was then repeated 5 times. The collected supernatant was centrifuged in an ultra-centrifuge (Beckman) at 4° C. for 55 min at 49,000×g. Then, the pellet was re-suspended in 10 mM ice-cold HEPES buffer (pH 7.5), aliquoted, and stored at −80° C. The protein concentration of those membrane suspensions was determined by the Bradford method, BSA as the standard.

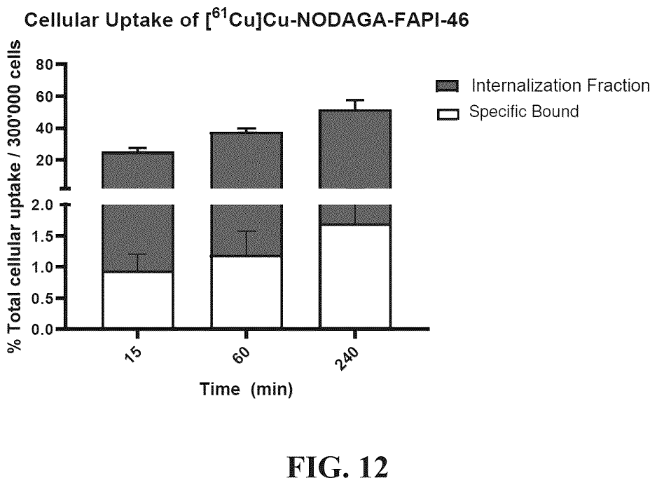

Saturation Experiment: The association profiles of ⁶¹Cu-labeled radioligands were studied at different concentrations, ranging from 0.075 to 50 nM, in HEK-293.hFAP cell membranes at 37° C. Each assay tube contained 170 μL of binding buffer (20 mM HEPES, pH 7.4, containing 4 mM MgCl₂, 0.2% BSA, 20 mg/L bacitracin, 20 mg/L PMSF and 200,000 KIU/L aprotinin). The incubation was initiated by adding 30 μL of radioligand solution at 10 times the final concentration and 100 μL of cell membrane suspension to yield 10 g of protein per well. For the determination of the non-specific binding, 140 μL of the above binding buffer was added along with 30 μL of FAPI-46 to obtain (0.1 mM). Bound fractions were plotted versus the corresponding radioligand concentration at equilibrium. The dissociation constant (KD) and maximal binding capacity (Bmax) values were calculated using GraphPad Software Inc., Prism 7, San Diego, CA, USA (Table 23 and FIG. 13).

TABLE 23
In Vitro Saturation Binding

| Compound | Bₘₐₓ | K_{D} (nM) |
|---|---|---|
| [⁶¹Cu]Cu-NODAGA-1 | 8.6-9.2 | 1.7-2.2 |
| [⁶¹Cu]Cu-NODAGA-3 | 7.3-8.1 | 1.2-1.8 |
| [⁶¹Cu]Cu-NODAGA-2 | 7.7-8.4 | 1.4-2.0 |
| [⁶¹Cu]Cu-NODAGA-4 | 9.1-9.8 | 3.0-3.9 |
| [⁶¹Cu]-NODAGA-FAPI-46 | 9.0-10.3 | 2.3-3.8 |

### 5.10. Example 10: Mice Studies

All animal experiments were conducted in accordance with Swiss animal welfare laws and regulations under the license number 30515 granted by the Veterinary Office (Department of Health) of the Canton Basel-Stadt.

Tumor Implantation: Female athymic nude-Foxn1nu/Foxn1+ mice (Envigo, The Netherlands), 4-6 weeks old, were injected subcutaneously with 5-10×10⁶ of HT-1080.hFAP cells suspended in 100 μL of PBS on the right shoulder or on the right flank, while 5-10×10⁶ HT-1080.wild-type cells suspended in 100 μL of PBS were injected on the contralateral shoulder or flank. The tumors were allowed to grow to an average volume of 100-200 mm³.

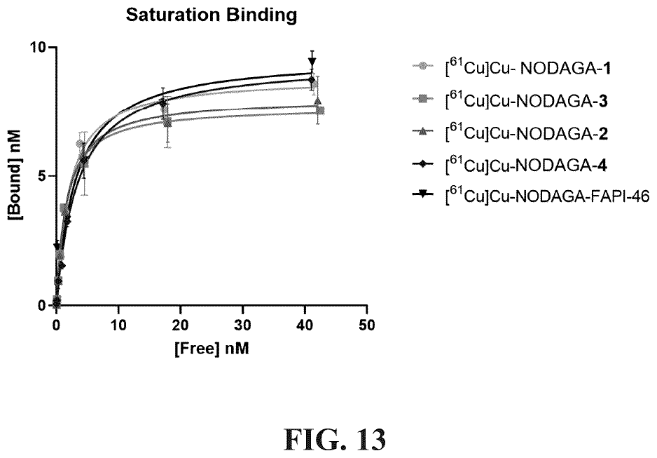

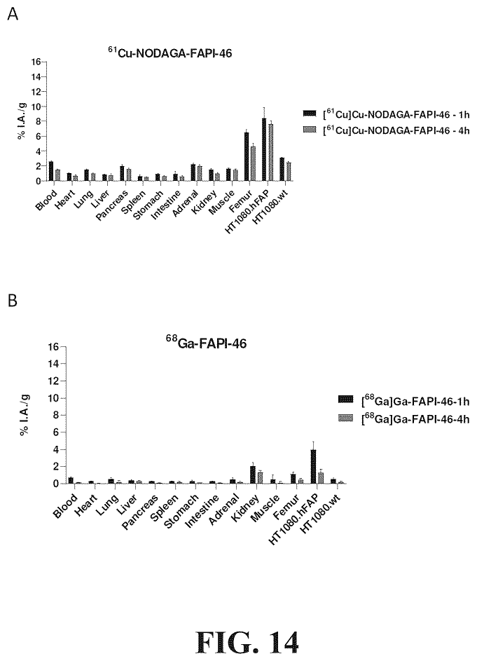

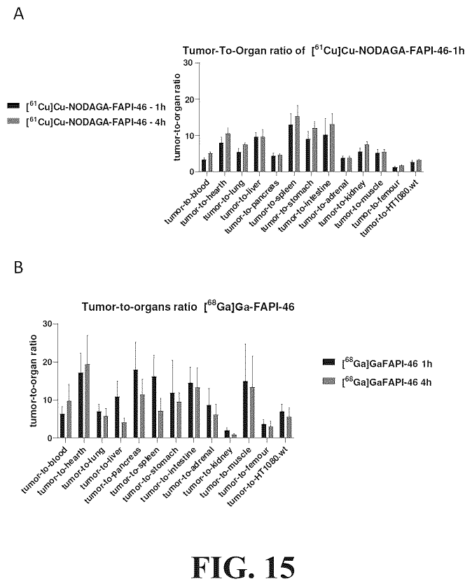

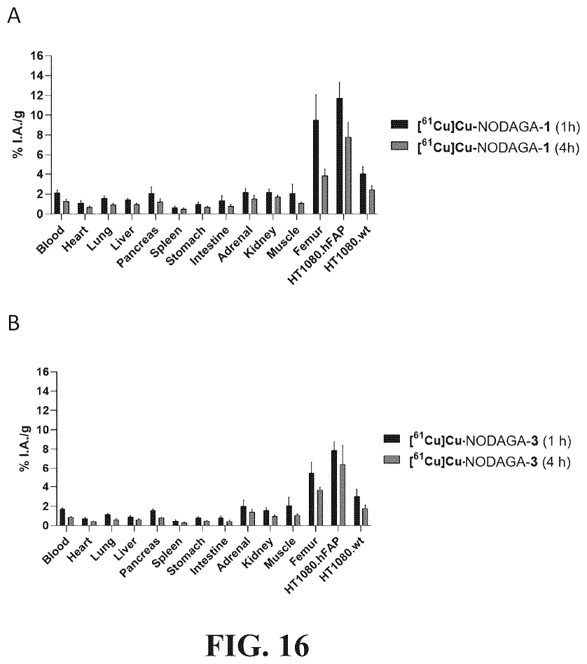

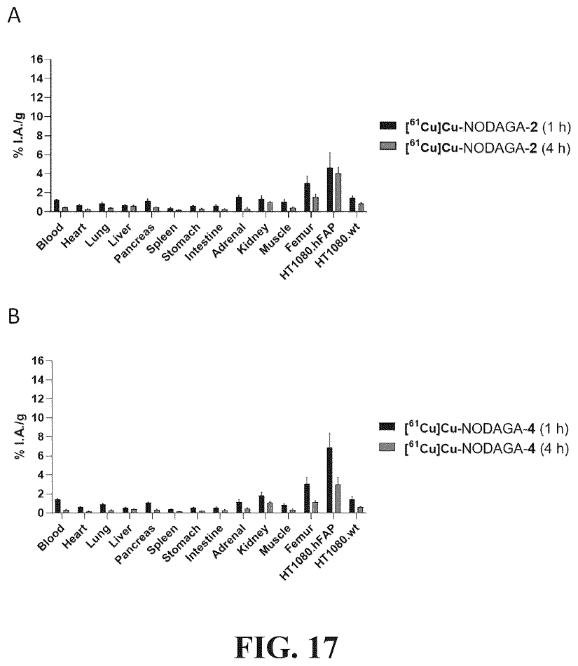

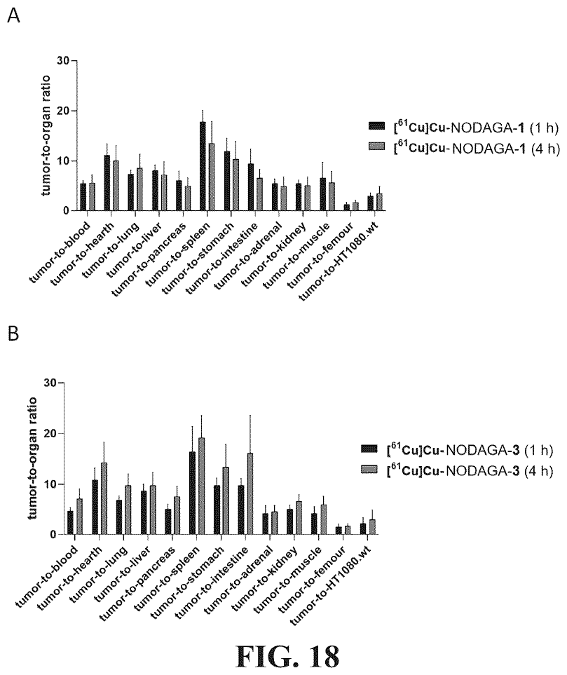

Biodistribution Studies: The xenografted mice were randomized (n=5 per group) and injected intravenously via the tail vein with the ⁶¹Cu-labeled radioligands (100 μL, 500 μmol, 0.8-1 MBq). Mice were euthanized 1 h and 4 h p.i. by CO₂ asphyxiation. Organs of interest and blood were collected, rinsed of excess blood, blotted dry, weighed, and counted in a γ-counter. The samples were counted against a suitably diluted aliquot of the injected solution as the standard and the results are expressed as the percentage of the injected activity per gram of tissue (% I.A./g)±SD. Results are shown in Table 24A-B and FIGS. 14-19.

TABLE 24A
Biodistribution data

| | [⁶¹Cu]Cu-NODAGA-1 | | [⁶¹Cu]Cu-NODAGA-2 | |
|---|---|---|---|---|
| Organ | 1 hour | 4 hours | 1 hour | 4 hours |
| Blood | 2.3 ± 0.1 | 1.2 ± 0.3 | 1.2 ± 0.1 | 0.5 ± 0.1 |
| Heart | 1.2 ± 0.3 | 0.7 ± 0.1 | 0.6 ± 0.1 | 0.3 ± 0.0 |
| Lung | 1.8 ± 0.0 | 0.8 ± 0.2 | 0.8 ± 0.1 | 0.4 ± 0.0 |
| Liver | 1.5 ± 0.0 | 1.0 ± 0.2 | 0.6 ± 0.1 | 0.6 ± 0.1 |
| Pancreas | 2.5 ± 0.3 | 1.4 ± 0.2 | 1.1 ± 0.2 | 0.5 ± 0.0 |
| Spleen | 0.8 ± 0.1 | 0.5 ± 0.1 | 0.4 ± 0.1 | 0.2 ± 0.0 |
| Stomach | 1.1 ± 0.2 | 0.7 ± 0.1 | 0.6 ± 0.1 | 0.3 ± 0.1 |
| Intestine | 1.7 ± 0.4 | 1.1 ± 0.4 | 0.5 ± 0.1 | 0.3 ± 0.1 |
| Adrenal | 2.3 ± 0.5 | 1.4 ± 0.2 | 1.5 ± 0.3 | 0.3 ± 0.1 |
| Kidneys | 2.4 ± 0.3 | 1.5 ± 0.5 | 1.1 ± 0.1 | 1.0 ± 0.1 |
| Muscle | 2.6 ± 0.7 | 1.3 ± 0.1 | 0.9 ± 0.1 | 0.4 ± 0.1 |
| Femur | 10.9 ± 1.1 | 4.2 ± 1.5 | 2.4 ± 0.3 | 1.6 ± 0.3 |
| HT-1080.hFAP | 12.6 ± 1.5 | 7.1 ± 3.0 | 3.5 ± 1.0 | 4.0 ± 0.6 |
| HT-1080.wt | 4.5 ± 0.6 | 2.1 ± 0.5 | 1.4 ± 0.2 | 0.9 ± 0.1 |
| Tumor mass | 0.1 ± 0.0 | 0.2 ± 0.2 | 0.4 ± 0.1 | 0.3 ± 0.1 |

TABLE 24B
Biodistribution data

| | [⁶¹Cu]Cu- | | [⁶¹Cu]Cu- | | [⁶¹Cu]Cu-NODAGA- | |
|---|---|---|---|---|---|---|
| | NODAGA-3 | | NODAGA-4 | | FAPI-46 | |
| Organ | 1 hour | 4 hours | 1 hour | 4 hours | 1 hr | 4 hours |
| Blood | 1.7 ± 0.1 | 0.9 ± 0.0 | 1.4 ± 0.1 | 0.4 ± 0.1 | 2.6 ± 0.1 | 1.5 ± 0.1 |
| Heart | 0.7 ± 0.1 | 0.5 ± 0.0 | 0.6 ± 0.0 | 0.2 ± 0.0 | 1.1 ± 0.0 | 0.7 ± 0.1 |
| Lung | 1.2 ± 0.1 | 0.7 ± 0.1 | 0.9 ± 0.1 | 0.3 ± 0.0 | 1.5 ± 0. | 1.0 ± 0.1 |
| Liver | 0.9 ± 0.1 | 0.7 ± 0.1 | 0.6 ± 0.1 | 0.4 ± 0.0 | 0.9 ± 0.0 | 0.8 ± 0.2 |
| Pancreas | 1.6 ± 0.2 | 0.9 ± 0.0 | 1.1 ± 0.1 | 0.4 ± 0.1 | 2.0 ± 0.2 | 1.6 ± 0.1 |
| Spleen | 0.5 ± 0.1 | 0.3 ± 0.0 | 0.4 ± 0.0 | 0.2 ± 0.0 | 0.7 ± 0.1 | 0.5 ± 0.1 |
| Stomach | 0.8 ± 0.1 | 0.5 ± 0.0 | 0.5 ± 0.0 | 0.2 ± 0.0 | 1.0 ± 0.1 | 0.6 ± 0.1 |
| Intestine | 0.8 ± 0.2 | 0.4 ± 0.1 | 0.5 ± 0.2 | 0.3 ± 0.1 | 1.0 ± 0.4 | 0.6 ± 0.2 |
| Adrenal | 2.1 ± 0.6 | 1.4 ± 0.3 | 1.1 ± 0.2 | 0.5 ± 0.1 | 2.2 ± 0.1 | 2.0 ± 0.2 |
| Kidneys | 1.6 ± 0.3 | 1.0 ± 0.2 | 1.8 ± 0.2 | 1.1 ± 0.1 | 1.5 ± 0.1 | 1.0 ± 0.1 |
| Muscle | 2.1 ± 0.8 | 1.1 ± 0.1 | 0.8 ± 0.2 | 0.4 ± 0.1 | 1.7 ± 0.1 | 1.4 ± 0.2 |
| Femur | 5.4 ± 1.2 | 3.7 ± 0.3 | 3.2 ± 0.8 | 1.2 ± 0.1 | 6.5 ± 0.4 | 4.6 ± 0.4 |
| HT- | 7.9 ± 0.9 | 6.4 ± 2.0 | 7.4 ± 1.6 | 3.0 ± 0.8 | 8.4 ± 1.4 | 7.7 ± 0.4 |
| 1080.hFAP | | | | | | |
| HT-1080.wt | 4.0 ± 2.2 | 1.8 ± 0.3 | 1.7 ± 0.2 | 0.6 ± 0.0 | 3.1 ± 0.0 | 2.5 ± 0.2 |
| Tumor | 0.1 ± 0.0 | 0.1 ± 0.1 | 0.3 ± 0.1 | 0.3 ± 0.1 | 0.1 ± 0.0 | 0.2 ± 0.0 |
| mass | | | | | | |

[⁶¹Cu]Cu-NODAGA-1, [⁶¹Cu]Cu-NODAGA-2, [⁶¹Cu]Cu-NODAGA-3, [⁶¹Cu]Cu-NODAGA-4, and [⁶¹Cu]Cu-NODAGA-1 showed high accumulation in FAP-positive (HT-1080.hFAP) tumor and murine-FAP-positive tissues, such as the bone marrow (bones).

PET/CT Imaging: Mice bearing FAP-positive and FAP-negative xenografts were injected intravenously with ⁶¹Cu-labeled radioligands of the present disclosure or [⁶¹Cu]Cu-NODAGA-FAPI-46 (100 μL/500 pmol/6-12 MBq). Mice were anesthetized with 1.5% isoflurane and dynamic PET scans were acquired during 1 hour upon injection of the radiotracer. The mice were euthanized by CO₂ at 4 hours p.i., and static PET scans were acquired for 30 min.

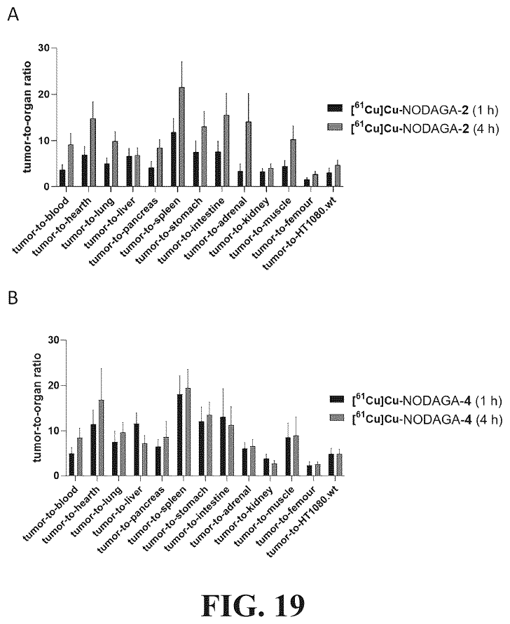

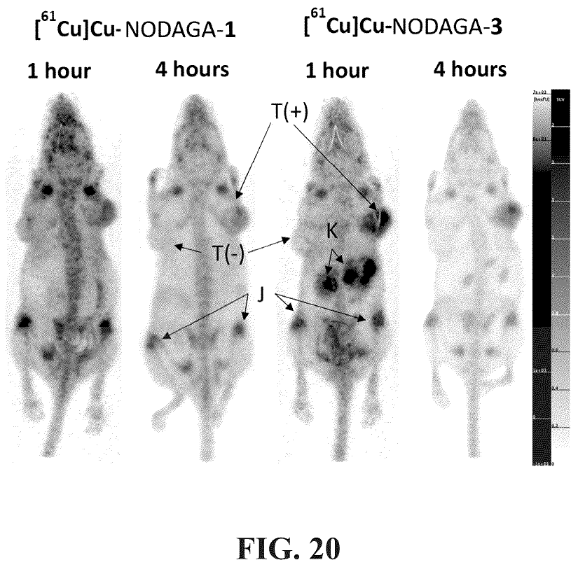

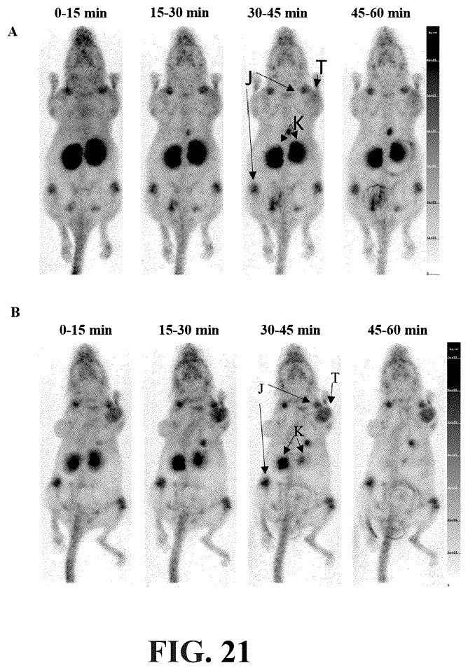

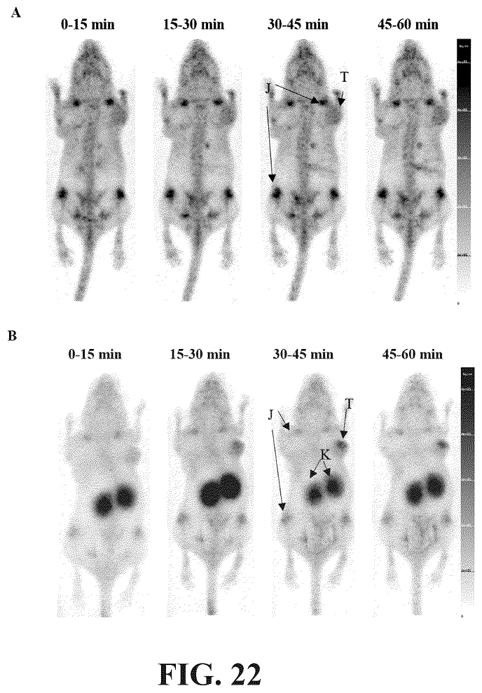

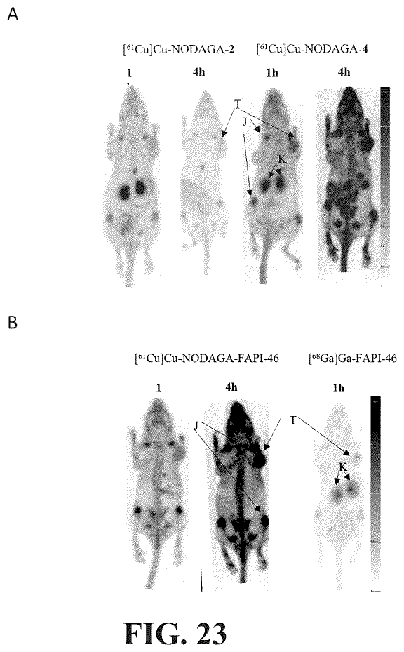

PET/CT images were acquired using β-CUBE PET scanner system (Molecubes, Gent, Belgium), with a spatial resolution of 0.85 mm and an axial field-of-view of 13 cm. Dynamic PET scans were acquired for 60 min. All PET scans were decay corrected and reconstructed into a 192×192×384 matrix by an ordered subsets maximization expectation (OSEM) algorithm using 30 iterations, a voxel size of 400×400×400 m a 15 min per frame. CT data was used to apply attenuation correction on the PET data. The CT was imaged supine, head first, using the NanoSPECT/CT™ scanner (Bioscan Inc.). Topograms and helical CT scans of the whole mouse were first acquired using the following parameters: X-ray tube current: 177 μA, X-ray tube voltage 45 kVp, 90 seconds and 180 frames per rotation, pitch 1. CT images were reconstructed using CTReco (version r1.146), with a standard filtered back projection algorithm (exact cone beam) and post-filtered (RamLak, 100% frequency cut-off), resulting in a pixel size of 0.2 mm. Co-registered PET/CT images were visualized using maximum intensity projection (MIP) with VivoQuant software (version 4.0). (FIGS. 20-24).

Remaining PET activity in the mouse body 4 h p.i. prior to the 4 h scan was determined (Table 25). [⁶¹Cu]Cu-NODAGA-FAPI-46 and [⁶¹Cu]Cu-NODAGA-1 showed the highest retention in the body, while [⁶¹Cu]Cu-NODAGA-4 presented the lowest value. Due to the physical characteristic of the radionuclide, [⁶⁸Ga]Ga-FAPI-46 was not evaluated 4 h p.i.

TABLE 25
In Vitro PET Remaining Activity

| | Injected | Activity | |
|---|---|---|---|
| | Activity | left after | Percentage of |
| | (MBq) | 4 h (MBq) | activity left |
| [⁶¹Cu]Cu-NODAGA-1 | 10.07 | 1.85 | 18.4% |
| [⁶¹Cu]Cu-NODAGA-3 | 7.65 | 0.86 | 11.2% |
| [⁶¹Cu]Cu-NODAGA-2 | 12.12 | 1.31 | 10.8% |
| [⁶¹Cu]Cu-NODAGA-4 | 10.34 | 0.76 | 7.4% |
| [⁶¹Cu]Cu-NODAGA-FAPI-46 | 7.25 | 1.34 | 18.9% |
| [⁶⁸Ga]Ga-FAPI-46 | 12.23 | / | / |

### 5.11. Example 11: Exemplary Method of Measuring Apparent Molar Activity

A [⁶¹Cu]CuCl₂ solution may be characterized by apparent molar activity (AMA). Apparent molar activity assesses, for example, competing trace metal impurities of ⁶¹Cu chelation by quantifying the minimum amount of a given chelator required for efficient radiolabeling.

An AMA test measures the percentage of complexation, by radio-TLC, after titrating a quantity of [⁶¹Cu]CuCl₂ solution with different amounts of a chelator (e.g., DOTA, NODAGA, etc.).

The results were then plotted (x-axis (logarithmic): nmol of chelator; y-axis: % of complexation). The lowest nmol value of chelator corresponding to ≥95% complexation achieved was recorded.

The experimental AMA value was then calculated according to the formula:

$$AMA value ( experimental ) = \frac{Activity [ MBq ]}{{n}_{N O D A G A} [ nmol ]}$$

where:
Activity: indicates the activity present in the fixed amount of [⁶¹Cu]CuCl₂ solution used for the titration decay corrected at the EoP (end of production; end of bombardment plus 1 hour).
n_{NODAGA}: indicates the lowest nmol value of chelator for which ≥95% complexation was achieved.

This test was performed to assess, for example:
1) the grade of a chemical employed for the manufacturing of the [⁶¹Cu]CuCl₂ solution;
2) the quality of a consumable employed for the manufacturing of the [⁶¹Cu]CuCl₂ solution;
3) the target coin manufacturing process; or
4) in case poor radiolabeling yields were observed.

### AMA Test Procedure

The mobile phase was prepared as (0.1M sodium citrate) as follows:
   Weigh 5.882 g of trisodium citrate dihydrate.
   Add Suprapur water up to a volume of 200 mL.
   Adjust the pH by adding HCl dropwise until it reaches a pH of 5.
Transfer an adequate amount of mobile phase into the TLC development chamber to cover a depth of 5 mm.
Prepare the sodium acetate solution (0.5 M in Ultrapur water) as follows:
   Weigh 2.051 g of sodium acetate.
   Add Ultrapur water up to a volume of 50 mL.
   Apply 10 μL of the solution on a pH strip and check if the pH was 8.
A 5 mM stock solution of NODAGA was prepared in 0.5 M sodium acetate (Stock 1) as follows:
   Weigh 3.1 mg of NODAGA.
   Add Ultrapur water or equivalent up to a volume of 1.5 mL.
   Store at −20° C. after use.
A 50 μM stock solution of NODAGA was prepared in 0.5 M sodium acetate (Stock 2) by mixing 10 μL of Stock 1 with 990 μL 0.5 M sodium acetate. Store at −20° C. after use.
NODAGA test solutions S1-S2 were prepared for chelator titration as displayed in Table T1 by diluting Stock 2 with the listed volumes of sodium acetate 0.5 M. The test solution S0 was a blank solution (without the addition of NODAGA) of 100 μL of 0.5 M sodium acetate.

TABLE T1
Preparation of solution 1-3 for chelator titration.

| | | | Volume |
|---|---|---|---|
| | | Volume | sodium |
| Solution | c_{NODAGA} | stock 2 | acetate |
| name | [nM] | [μL] | [μL] |
| S0 | 0 | 0 | 100 |
| S 1 | 3000 | 100 | 1567 |
| S 2 | 1500 | 50 | 1617 |
| S 3 | 900 | 30 | 1637 |

The test solutions, S4-S10, 1:10 dilutions of the solutions S1-S7, were prepared with 0.5 M sodium acetate according to the dilution scheme shown in Table T2.

TABLE T2
Preparation of solution 4-10 for chelator titration.

| | | | Volume |
|---|---|---|---|
| | | | sodium |
| Solution | c_{NODAGA} | Volume | acetate |
| name | [nM] | Sx [μL] | [μL] |
| S 4 | 300 | 100 of S1 | 900 |
| S 5 | 150 | 100 of S2 | 900 |
| S 6 | 90 | 100 of S3 | 900 |
| S 7 | 30 | 100 of S4 | 900 |
| S 8 | 15 | 100 of S5 | 900 |
| S 9 | 9 | 100 of S6 | 900 |
| S 10 | 3 | 100 of S7 | 900 |

A solution of 0.05 M HCl was prepared as follows:
   Add 5 mL of Ultrapur water in a 15 mL Falcon tube.
   Add 53 μL of 30% HCl to the falcon using a micropipette.
   Add Ultrapur water up to 10 mL.
   Spot 10 μL of the solution on a pH strip and confirm the pH is between 1.0 and 1.6.
The [⁶¹Cu]CuCl₂ solution was diluted with 0.05 M hydrochloric acid to reach a total volume of 1 mL with an activity concentration of 0.2 MBq/μL (EoP) according to the following formula:

$$[ {}^{61} Cu ] {CuCl}_{2} solution [ μL ] = \frac{0.2 [ MBq / μL ]}{A C ( E o P ) [ MBq / μL ]} * 1000 μL$$

0.05 M hydrochloric acid for dilution=1000 μL−[⁶¹Cu]CuCl₂ solution to draw [μL] where AC (EoP) is the activity concentration at EoP.
The solution were named “diluted Cu-61 solution”. See the examples in Table T3.

TABLE T3
[⁶¹Cu]CuCl₂ dilution examples.

| | | Activity | |
|---|---|---|---|
| Activity | | concentration | |
| eluted at | Volume | at EoP | |
| EoP [MBq] | [mL] | [MBq/μL] | Dilution for AMA test |
| 1000 | 3.0 | 0.33 | 600 μL of [⁶¹Cu]CuCl₂ |
| | | | diluted with 400 μL of |
| | | | 0.05M HCl |
| 2000 | 3.0 | 0.67 | 300 μL of [⁶¹Cu]CuCl₂ |
| | | | diluted with 700 μL of |
| | | | 0.05M HCl |
| 3500 | 3.0 | 1.17 | 233 μL of [⁶¹Cu]CuCl₂ |
| | | | diluted with 767 μL of |
| | | | 0.05M HCl |

Add in each solution for chelator titration 50 μL of diluted [⁶¹Cu]CuCl₂ solution (as prepared in Table T3), obtaining the reaction solutions listed in Table T4.

TABLE T4
Reaction solutions.

| | | Volume of | Volume | | | |
|---|---|---|---|---|---|---|
| Reaction | | NODAGA | of ⁶¹Cu | Finale | | |
| solution | NODAGA | solution Sx | solution | volume | n_{NODAGA} | c_{NODAGA} |
| name | solution | [μL] | [μL] | [μL] | [nmol] | [nM] |
| RS 1 | S1 | 100 | 50 | 150 | 0.3 | 2000 |
| RS 2 | S2 | 100 | 50 | 150 | 0.15 | 1000 |
| RS 3 | S3 | 100 | 50 | 150 | 0.09 | 600 |
| RS 4 | S4 | 100 | 50 | 150 | 0.03 | 200 |
| RS 5 | S5 | 100 | 50 | 150 | 0.015 | 100 |
| RS 6 | S6 | 100 | 50 | 150 | 0.009 | 60 |
| RS 7 | S7 | 100 | 50 | 150 | 0.003 | 20 |
| RS 8 | S8 | 100 | 50 | 150 | 0.0015 | 10 |
| RS 9 | S9 | 100 | 50 | 150 | 0.0009 | 6 |
| RS 10 | S10 | 100 | 50 | 150 | 0.0003 | 2 |
| RS 0 | S0 | 100 (Only 100 | 50 | 150 | 0 | 0 |
| | | μL of sodium | | | | |
| | | acetate 0.5M) | | | | |

The reaction solutions were incubated at room temperature for 5 minutes.
After incubation, perform TLC measurement on all the reaction solutions as follows:
   Using a micropipette, spot 2 μL of each [⁶¹Cu]Cu-NODAGA reaction solution RS0-RS10 onto different pre-cut TLC paper.
   Allow the TLC paper to dry for about 5 minutes or until there was no visible stain.
   Place the TLC paper in the TLC chamber with the stained side on the bottom of the chamber. When the solvent front was about 1 cm from the top of the strip, remove the TLC paper from the TLC chamber with tweezers.
   Mark the solvent front on the TLC paper strip with a pencil.
   Measure the distance between the starting line and the solvent front with a ruler and let the strip dry completely.
After scanning the TLC plate, the software (e.g., Biochrom) will generate an evaluation report. Print the evaluation report and record the results. All data (i.e., RF, % of total activity, % of ROI) of the peaks must be documented.
Using the regions of interest (ROI) technique (drawing regions over distinct areas of activities), the % of complexation of [⁶¹Cu]Cu-NODAGA was expressed as a percentage of the total detected activity, as follows:

$${Complexation}_{[ 61 Cu ] Cu - N O D A G A} ( \% ) = \frac{{Activity}_{[ {}^{61} Cu ] Cu - NODAGA} [ counts ]}{Total activity [ counts ]} * 100$$

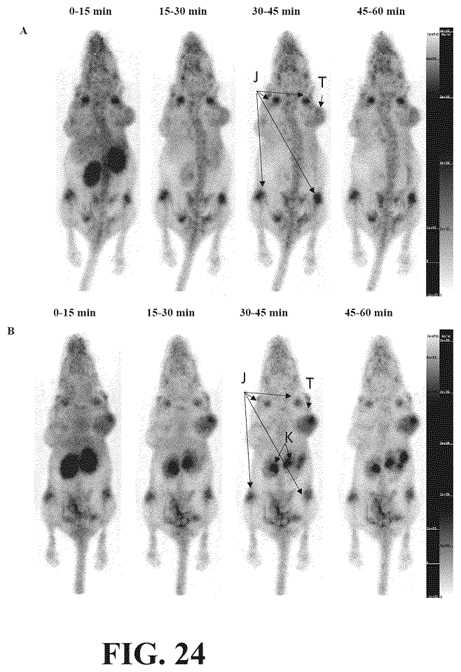

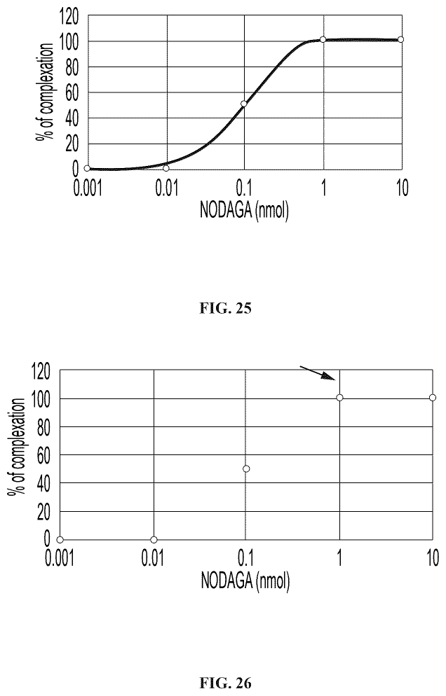

The TLC scanner results were plotted (x-axis (logarithmic): nmol of NODAGA chelator; y-axis: % of complexation), and will follow a sigmoidal trend. An example of plot was given in FIG. 25.
The lowest value of nmol of chelator in correspondence of which ≥95% complexation is identified on the plot. An example was reported in FIG. 26.

The experimental AMA value was then calculated according to the formula:

$$AMA value ( experimental ) = \frac{Activity [ MBq ]}{{n}_{N O D A G A} [ nmol ]}$$

where:
Activity: indicates the activity present in the fixed amount of [⁶¹Cu]CuCl₂ solution used for the titration decay corrected at the EoP.
n_{NODAGA}: indicates the lowest value of nmol of chelator where ≥95% complexation was achieved.

### 6. EQUIVALENTS AND INCORPORATION BY REFERENCE

While aspects of this disclosure have been particularly shown and described with reference to a preferred embodiment and various alternate embodiments, it will be understood by persons skilled in the relevant art that various changes in form and details can be made therein without departing from the scope of the disclosure.

All references, issued patents, and patent applications cited within the body of the instant specification are hereby incorporated by reference in their entirety, for all purposes. In particular, U.S. Provisional Application No. 63/568,863 (filed Mar. 22, 2024) and International Application Nos. PCT/US2023/75064 (filed Sep. 25, 2023), PCT/US2023/75067 (filed Sep. 25, 2023), PCT/US2023/075066 (filed Sep. 25, 2023), and PCT/US2025/21148 (filed Mar. 24, 2025) are hereby incorporated by reference in their entirety.

1. A method for imaging a subject comprising:
(a) administering an effective amount of a pharmaceutical composition to a subject suspected of having or diagnosed with a disorder associated with expression of fibroblast activation protein (FAP), wherein the pharmaceutical composition comprises:
i. a radiotracer that has one of the following structures

| Radiotracer | Structure |
|---|---|
| 61Cu-NODAGA-1 | 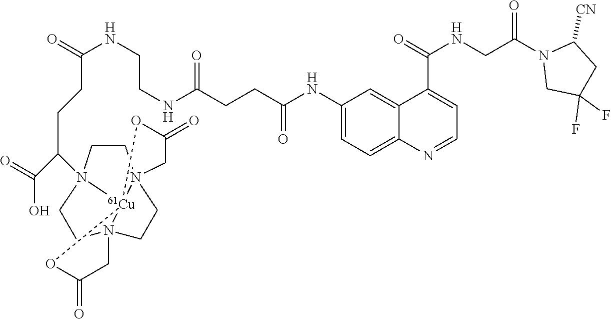 |
| ⁶¹Cu-NODAGA-2 | 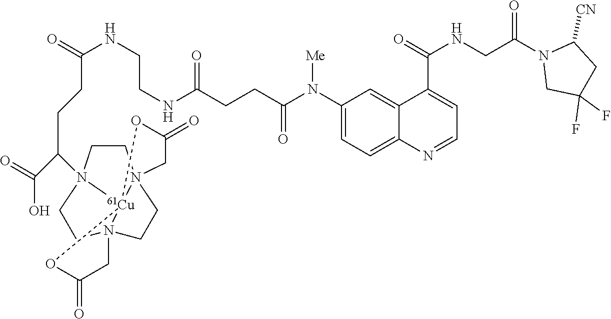 |
| ⁶¹Cu-NODAGA-3 | 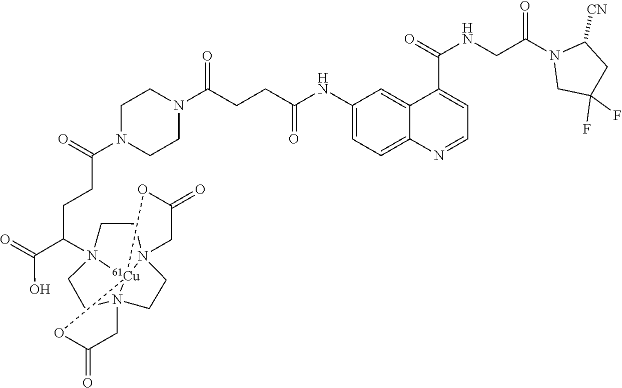 |
| ⁶¹Cu-NODAGA-4 | 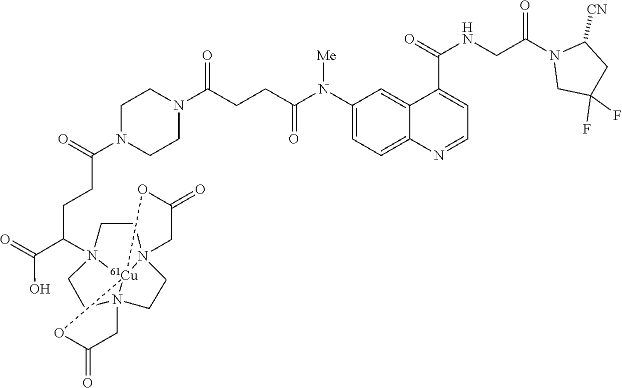 |

or is a pharmaceutically acceptable salt thereof; and
ii. a radiolytic inhibitor;
wherein the pharmaceutical composition is characterized by one or more of:
a [⁶¹Cu]Cu radionuclidic purity at end of synthesis of ≥97%,
a radiocobalt activity content at end of synthesis of ≤0.05%,
a ^{110m}Ag specific activity ≤0.1 Bq/g,
a ^{108m}Ag specific activity ≤0.1 Bq/g, or
a ¹⁰⁹Cd specific activity ≤0.1 Bq/g; and
(b) generating one or more radiographic images of the subject.

2. (canceled)

3. A method for determining a subject's response to a cancer treatment comprising:
(a) administering an effective amount of a pharmaceutical composition to a subject diagnosed with a fibroblast activation protein (FAP)-expressing cancer or tumor at an earlier time point and at a later time point, wherein the pharmaceutical composition comprises:
i. a radiotracer that has one of the following structures

| Radiotracer | Structure |
|---|---|
| ⁶¹Cu-NODAGA-1 | 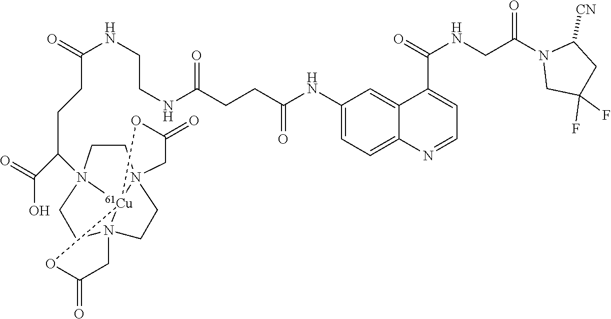 |
| ⁶¹Cu-NODAGA-2 | 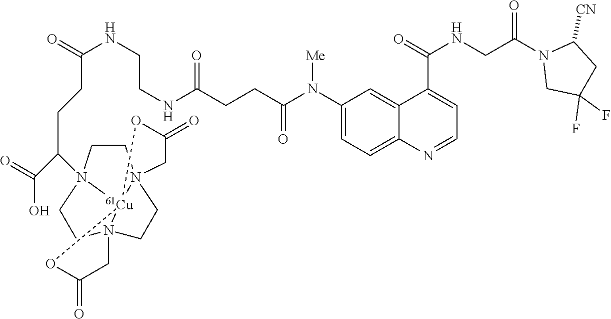 |
| ⁶¹Cu-NODAGA-3 | 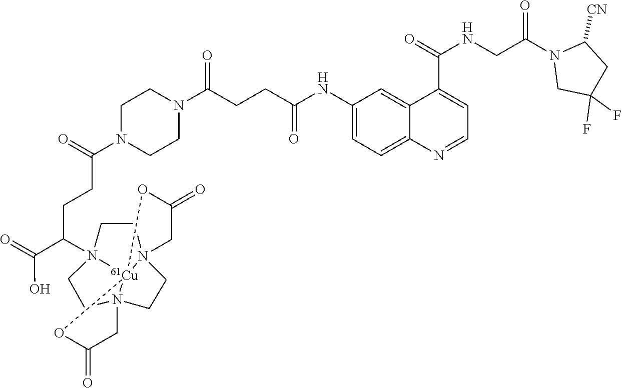 |
| ⁶¹Cu-NODAGA-4 | 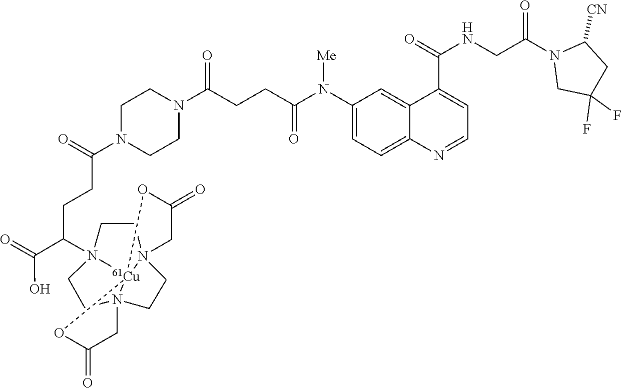 |

or is a pharmaceutically acceptable salt thereof; and
ii. a radiolytic inhibitor;
wherein the pharmaceutical composition is characterized by one or more of:
a [⁶¹Cu]Cu radionuclidic purity at end of synthesis of ≥97%,
a radiocobalt activity content at end of synthesis of ≤0.05%,
a ^{110m}Ag specific activity ≤0.1 Bq/g,
a ^{108m}Ag specific activity ≤0.1 Bq/g, or
a ¹⁰⁹Cd specific activity ≤0.1 Bq/g;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

4-54. (canceled)

55. A pharmaceutical composition comprising:
(a) a radiotracer that has one of the following structures

| Radiotracer | Structure |
|---|---|
| 61Cu-NODAGA-1 | 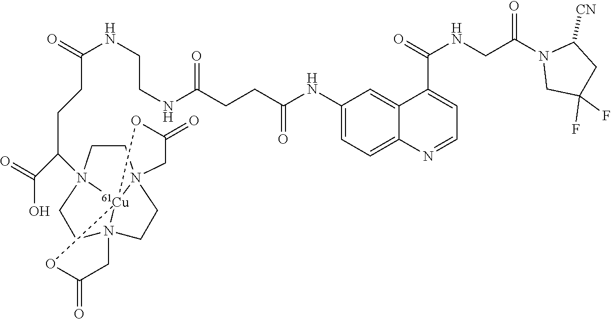 |
| ⁶¹Cu-NODAGA-2 | 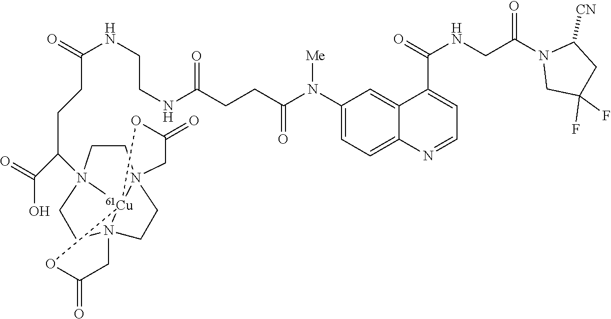 |
| ⁶¹Cu-NODAGA-3 | 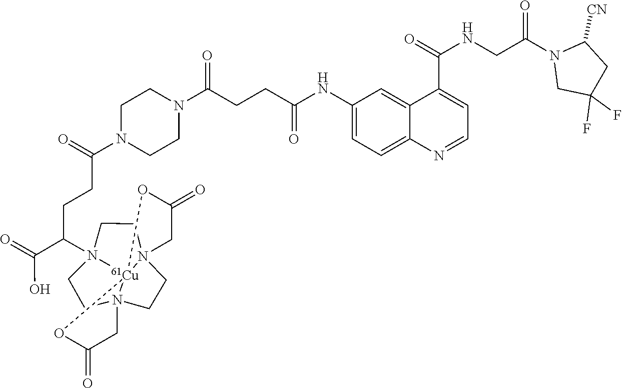 |
| ⁶¹Cu-NODAGA-4 | 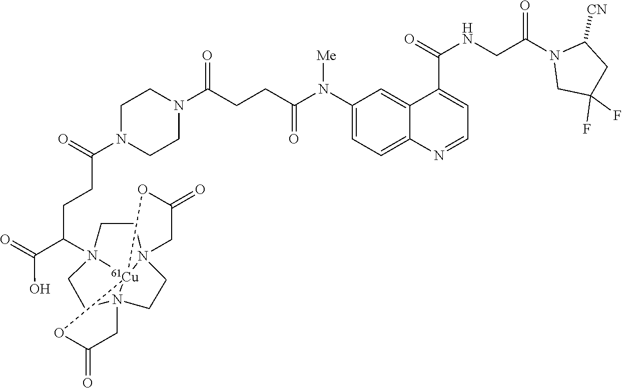 |

or is a pharmaceutically acceptable salt thereof,
(b) a radiolytic inhibitor, and
(c) an aqueous vehicle;
wherein the pharmaceutical composition is characterized by one or more of:
a [⁶¹Cu]Cu radionuclidic purity at end of synthesis of ≥97%,
a radiocobalt activity content at end of synthesis of ≤0.05%,
a ^{110m}Ag specific activity ≤0.1 Bq/g,
a ^{108m}Ag specific activity ≤0.1 Bq/g, or
a ¹⁰⁹Cd specific activity ≤0.1 Bq/g.

56. The pharmaceutical composition according to claim 55, wherein the activity of the pharmaceutical composition is ≥25 MBq.

57. (canceled)

58. The pharmaceutical composition according to claim 55, wherein the radiotracer radiochemical purity of the pharmaceutical composition is ≥90% at 12 hours after end of synthesis.

59. (canceled)

60. The pharmaceutical composition according to claim 55, wherein the pharmaceutical composition is characterized by a ⁶¹[Cu]Cu radionuclidic purity of the pharmaceutical composition at end of synthesis of ≥98%.

61. The pharmaceutical composition according to claim 55, wherein the pharmaceutical composition is characterized by a radiocobalt activity content at end of synthesis of ≤0.01%.

62. The pharmaceutical composition according to claim 55, wherein the pharmaceutical composition is characterized by at least one of a ⁵⁶[Co]Co specific activity or ⁵⁸[Co]Co specific activity of ≤1,500 Bq/g.

63-65. (canceled)

66. The pharmaceutical composition according to claim 55, wherein the pharmaceutical composition is characterized by one or more of: a ^{110m}Ag specific activity ≤0.1 Bq/g, a ^{108m}Ag specific activity ≤0.1 Bq/g, or a ¹⁰⁹Cd specific activity ≤0.1 Bq/g.

67. The pharmaceutical composition according to claim 55, wherein the sum of the specific activities of the radionuclidic impurities in the pharmaceutical composition is ≤8,000 Bq/g.

68. The pharmaceutical composition according to claim 55, wherein the pharmaceutical composition is characterized by one or more of: Al≤1.2 ng/MBq, Co≤0.2 ng/MBq, Fe≤1.7 ng/MBq, Pb≤0.8 ng/MBq, or Zn≤0.8 ng/MBq.

69. The pharmaceutical composition according to claim 55, wherein the activity concentration of the pharmaceutical composition is ≥10 MBq/mL.

70. (canceled)

71. The pharmaceutical composition according to claim 55, wherein the apparent molar activity of the radiotracer is ≥1 MBq/nmol.

72. The pharmaceutical composition according to claim 55, wherein the radiotracer is present in an amount ≥1 μg.

73. (canceled)

74. The pharmaceutical composition according to claim 55, wherein the aqueous vehicle comprises isotonic saline.

75. The pharmaceutical composition according to claim 55, wherein the radiolytic inhibitor is present in the pharmaceutical composition in a concentration of ≥1 mg/mL.

76. The pharmaceutical composition according to claim 55, wherein the radiolytic inhibitor is present in the pharmaceutical composition in a concentration from 1 mg/mL to 10 mg/mL.

77. The pharmaceutical composition according to claim 55, wherein the radiolytic inhibitor is selected from ascorbic acid, gentisic acid, citric acid, N-tert-butyl-α-phenylnitrone (PBN), polyvinylpyrrolidone (PVP), ethanol, DMSA, cysteine, vanillin, methionine, adenine, dobesilic acid, thymine, uracil, nicotinic acid, nicotinamide, salts of any of the foregoing, and combinations thereof.

78. The pharmaceutical composition according to claim 77, wherein the radiolytic inhibiter is ascorbic acid or a salt thereof.

79. The pharmaceutical composition according to claim 55, comprising:
1-100 μg of the radiotracer,
5-20 mg of ascorbic acid or a salt thereof, and
1-10 mL isotonic saline solution.

80. The pharmaceutical composition according to claim 55, wherein the composition is formulated for intravenous infusion.

81. The pharmaceutical composition of claim 55, wherein the total volume is from 2 mL to 15 mL.

82. The pharmaceutical composition according to claim 55, having ≥99% radiochemical purity at 3 hours after end of synthesis.

83. The pharmaceutical composition according to claim 55, wherein the pH is from 5 to 7.
